(19) United States
(12) Patent Application Publication
(10) Pub. No.: US 2007/0254863 A1
(43) Pub. Date: Nov. 1, 2007

(54) Use of CBx cannabinoid receptor modulators as potassium channel modulators

(76) Inventors: Jochen Antel, Bad Munder (DE); Peter-Colin Gregory, Hannover (DE); Josephus Hubertus Maria Lange, Almere (NL); Michael Firnges, Barsinghausen (DE); Dania Reiche, Adelheidsdorf (DE)

Correspondence Address: MAYER BROWN LLP, P.O. BOX 2828, CHICAGO, IL, 60690, US

(21) Appl. No.: 11/796,740

(22) Filed: Apr. 26, 2007

Related U.S. Application Data
(60) Provisional application No. 60/745,757, filed on Apr. 27, 2006.
(60) Provisional application No. 60/745,760, filed on Apr. 27, 2006.

(51) Int. Cl. A61K 31/55; A61K 31/5377; A61K 31/454

(52) U.S. Cl. 514/183; 514/365; 514/454; 514/323; 514/326; 514/235.5; 514/210.01

(57) ABSTRACT

Methods of treating one or more medical conditions by administering to a subject in need thereof an effective amount of a CBₓ modulator having K_{ATP} channel modulating properties are described herein. Also described are methods of using a CBₓ modulator having K_{ATP} channel modulating properties to treat one or more medical conditions.

### CROSS REFERENCE TO RELATED APPLICATIONS

This application claims the benefit of U.S. Provisional Application Nos. 60/745,757 and 60/745,760 which were both filed on Apr. 27, 2006 and are both hereby incorporated by reference in their entirety to the extent permitted by law.

### FIELD

Described herein is the novel use of CBₓ modulators as K_{ATP} channel modulators. Also disclosed herein are methods of treating one or more medical conditions, such as obesity, diabetes mellitus, metabolic syndrome, syndrome X, insulinoma, familial hyperinsulemic hypoglycemia, male pattern baldness, detrusor hyperreactivity, asthma, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain, idiopathic pain, and impotence in mammals, such as humans, comprising administering to a subject in need thereof an effective amount of a CBₓ modulator as K_{ATP} channel modulator.

### BACKGROUND

As used herein, obesity is meant to comprise any increase in body fat that results in increased bodyweight, preferably comprising but not limited to the medical definition of obesity. Thus, obesity also comprises any body fat increase not meeting the medical definition of obese, e.g. cosmetically overweight. The present disclosure thus also relates to non-medical weight loss, such as cosmetic weight loss and includes improving body appearance in general. In one sense, obesity is understood to denominate a body weight more than 20% above the ideal body weight. In this sense, obesity is a major health concern in Western societies. It is estimated that about 97 million adults in the United States are overweight or obese. Obesity is largely the result of a positive energy balance as a consequence of increased ratio of caloric intake to energy expenditure. The molecular factors regulating food intake and body weight are not completely understood, but several genetic factors have been identified.

Epidemiological studies have shown that increasing degrees of overweight and obesity are important predictors of decreased life expectancy. Obesity causes or exacerbates many health problems, both independently and in association with other diseases. The problems associated with obesity, which can be serious and life-threatening, are well-known and generally include hypertension; type II diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arteriosclerosis; heart disease; abnormal heart rhythms; and heart arrythmias. Obesity is further associated with premature death and with a significant increase in mortality and morbidity from stroke, myocardial infarction, congestive heart failure, coronary heart disease, and sudden death.

Obesity is often treated by encouraging patients to lose weight by reducing their food intake or by increasing their exercise level and therefore increasing their energy output. A sustained weight loss of 5% to 10% of body weight has been shown to improve the co-morbidities associated with obesity, such as diabetes and hypertension, and can lead to improvement of obesity-related conditions such as osteoarthritis, sleep apnea and pulmonary and cardiac dysfunction.

Weight loss drugs that are currently used in monotherapy for the treatment of obesity have limited efficacy and significant side effects. During chronic treatment periods of greater than six months the efficacy of most agents decreases yielding no more than 10% body weight loss compared to control. Obese humans can easily have a body mass of over 150 kg and would, therefore, need to lose more than 50% of their body mass to return to a normal body mass.

The term “metabolic syndrome” is meant to cover a complex of clinical manifestations which, besides central obesity, mainly comprises hypertension, in particular arterial hypertension; insulin resistance, in particular type II diabetes; glucose intolerance; dyslipoproteinaemia, in particular as hypertriglyceridaemia, accompanied by dyslipoproteinaemia occurring with lowered HDL-cholesterol, and also hyperuricaemia, which can lead to gout.

According to information from the American Heart Association, metabolic syndrome is closely rinked to insulin resistance. Some people are genetically predisposed to insulin resistance. Acquired factors, such as excess body fat and physical inactivity, can elicit insulin resistance and metabolic syndrome in these people. Many people with insulin resistance have central obesity. The biological mechanisms at the molecular level between insulin resistance and metabolic risk factors are not fully understood and appear to be complex. One group of people at risk for developing metabolic syndrome is those with diabetes who have a defect in insulin action and cannot maintain a proper level of glucose in their blood. Another is people, mainly those with high blood pressure, who are non-diabetic and insulin-resistant but who compensate by secreting large amounts of insulin. This condition is known as hyperinsulinemia. A third group is heart attack survivors who, unlike hypertensives, have hyperinsulinemia without having abnormal glucose levels. Metabolic syndrome has become increasingly common in more developed countries like the United States, where it is estimated that between about 20% and 25% of US adults have metabolic syndrome. There are no well-accepted criteria for diagnosing the metabolic syndrome. The criteria proposed by the Third Report of the National Cholesterol Education Program (NCEP) Expert Panel on Detection, Evaluation, and Treatment of High Blood Cholesterol in Adults (Adult Treatment Panel III) are the most current and widely used. According to the Adult Treatment Panel III criteria, the metabolic syndrome is identified by the presence of three or more of these following components:
a. Central obesity as measured by waist circumference (Men—Greater than 40 inches; Women—Greater than 35 inches).
b. Fasting blood triglycerides greater than or equal to 150 mg/dL.
c. Blood HDL cholesterol (Men—Less than 40 mg/dL; Women—Less than 50 mg/dL)
d. Blood pressure greater than or equal to 130/85 mmHg.
e. Fasting glucose greater than or equal to 110 mg/dL.

The term “syndrome X” is closely related to the term “metabolic syndrome” and is typically used to indicate the identical disease or condition. According to information from the American Heart Association, the term “Syndrome X” refers, however, additionally to a heart condition where chest pain and electrocardiographic changes that suggest ischemic heart disease is present, but where there are no angiographic findings of coronary disease. Patients with cardiac syndrome X also sometimes have lipid abnormalities.

Therefore, one embodiment described herein provides a more effective and/or more selective therapy for obesity, diabetes mellitus, metabolic syndrome, syndrome X, insulinoma, familial hyperinsulemic hypoglycemia, male pattern baldness, detrusor hyperreactivity, asthma, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain idiopathic pain and impotence in mammals, such as humans.

ATP-sensitive potassium channel (K_{ATP} channel) modulation has been linked to several potential clinical uses including diabetes, insulinoma, familial hyperinsulemic hypoglycemia, male pattern baldness, detrusor hyperreactivity, asthma, neuroprotection, epilepsy, analgesia, cardioprotection, angina, cardioplegia, arrhythmia, coronary spasm, hypertension, peripheral vascular disease, cerebral vasospasm, appetite regulation, neurodegeneration, pain—including neuropathic pain and chronic pain—and impotence (ref. Jahangir et al. J. Mol. Cell. Cardiology, 2005, 39, 99-112 and references cited therein).

K_{ATP} channel openers and their potential use in the inhibition of insulin secretion and/or the treatment of metabolic disorders are discussed in U.S. Pat. No. 6,492,130; WO 02/00223; WO 02/00665; R. D. Carr et al., Diabetes 52 (2003) 2513-2518; and J. B. Hansen et al., Current Medicinal Chemistry 11 (2004) 1595-1615.

The beneficial role of the specific K_{ATP} channel opener diazoxide in the treatment of i.a. metabolic syndrome is discussed in U.S. Pat. No. 5,284,845; U.S. Pat. No. 6,197,765; R. Alemzadeh et al., Endocrinology 133 (2) (1993) 705-712; and R. Alemzadeh et al., Journal of Clinical Endocrinology and Metabolism 83 (6) (1998) 1911-1915.

The K_{ATP} channel couples glucose metabolism to insulin secretion. Defective regulation of K_{ATP} channel activity has been reported to contribute to the etiology of type 2 diabetes (ref. Ashcroft, J. Clin. Investig. 2005, 115 (8), 2047-2057 and references cited therein). The K_{ATP} channel is an octameric complex of 4 Kir6.x (x=1 or 2) and 4 regulatory SURy subunits (Y=1, 2A or 2B). The SUR1 regulatory subunit is in particular found in the human pancreas and brain (ref. Aguilar-Bryan et al., Science 1995, 268, 423-426). The K_{ATP} Kir6:2/SUR1 combination exists in the pancreas. Its structure has been determined recently (ref. Mikhailov, EMBO Journal, 2005, 24, (23), 4166-4175). Recent advances in the discovery of ATP-sensitive potassium channel openers have been reviewed (Pirotte et al., Exp Opin. Ther. Patents 2005, 15 (5), 497-504).

Insulin is the main hormone involved in blood glucose homeostasis. Insulin is involved in the regulation of glycaemia and as a consequence related to type I and type II diabetes. Additionally, insulin is involved in lipogenesis and weight gain, provoking an anorexigenic action as it provokes satiety when acting in the brain (ref. Juan-Pico et al., Cell Calcium 2006, 39, 155-163 and references cited therein).

Therefore, the regulation of insulin secretion will be useful in the treatment of diseases such as diabetus mellitus type I, diabetus mellitus type II, obesity, metabolic syndrome and syndrome X.

The endocannabinoid system (refs. (a) De Petrocellis, L. et al., Br. J. Pharmacol. 2004 141, 765-774; (b) Di Marzo, V. et al., Nature Rev. Drug Discov. 2004, 3, 771-784; (c) Lambert, D. M. and Fowler, C. J. J. Med. Chem. 2005, 48, 5059-5087) has been reported to play a role in the physiological regulation of food intake, energy balance and glucose and lipid metabolism. The existence of both cannabinoid CB₁ and CB₂ receptors has been demonstrated in the endocrine pancreas. It has been reported that the endogenous CB_{1/2} receptor agonist 2-arachidonoyl glycerol (2-AG) (FIG. 2) through CB₂ receptors regulates [Ca²⁺]ᵢ signals in β-cells in the endocrine pancreas and, as a consequence (as was concluded by Juan-Pico et al.), it decreases insulin secretion (ref. Juan-Pico et al., Cell Calcium 2006, 39, 155-163). Recent advances in the field of CB₂ receptor ligands have been reviewed by Raitio et al. (Curr. Med. Chem. 2005, 12, 1217-1237).

Receptors in a pharmacological sense are typically understood to be intracellular or membrane-bound proteins capable of producing a pharmacological effect after becoming bound with a specific ligand. In view of this, a pharmacological receptor has at least two genreal functions, first, to detect a ligand signal by forming a ligand-receptor complex and second, to conduct and translate the signal leading to the pharmacological effect.

It is understood that exogenous compounds (e.g. pharmaceutical drug products) can replace endogenous physiological ligands to bind, or otherwise interact, with pharmacological receptors. A required condition for such a drug-receptor interaction to occur is the formation of a drug-receptor complex. In contrast to physiological ligands that stimulate an response after binding to a receptor (receptor-mediated effects), drugs can be classified by the response which is stimulated. Thus, drugs, based upon its interaction with a particular receptor, can be characterized as (a) agonists which elicit or stimulate an effect after binding to the receptor, (b) partial agonists are compounds that elicit a biological response, but the response is less than that seen with an agonist, (c) antagonists are compounds which do not stimulate an effect after receptor binding, and (d) inverse agonists in which the basal activity of the receptor is decreased.

Several types of molecular bonding or attraction mechanisms are possible for drug-receptor binding and resulting formation of the drug-receptor complex. These molecular interactions include such phenomena as ionic and hydrogen bonding as well as van der Waals forces. Typical drug-receptor complexes involve several kinds of simultaneous binding. Ionic bonds are important aspects of drug-receptor interaction as these bonds are the strongest and have the longest range. After the initial interaction and complex formation, additional drug-receptor interactions occur and involve dipole-dipole-bonds, hydrogen bonds and hydrophobic bonds. Each of these bonding mechanisms tend to fix the drug with the receptor and are reversible, as the associated forces are weak. Thus, the pharmacological effectiveness of a particular active agent is usually affected by the blood plasma concentration. A decrease in plasma drug concentration would be expected to increase the dissociation of drug molecule from its receptor.

It has now surprisingly been found that the quantitative effects observed within the use of CBₓ modulators in the indications mentioned herein are greater than expected and, without being limited by a particular theory of operation, explainable by a single mechanism of CBₓ modulation. By a more thorough investigation, it is presently understood that CBₓ modulators act as K_{ATP} channel modulators. Hence, CBₓ modulators can be used for treating medical conditions which require opening of K_{ATP} channels. More specifically, CBₓ modulators can be used for treating one or more medical conditions, such as obesity, diabetes mellitus, metabolic syndrome, syndrome X, insulinoma, familial hyperinsulemic hypoglycemia, male pattern baldness, detrusor hyperreactivity, asthma, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain, idiopathic pain and impotence in mammals, such as humans.

### SUMMARY

One embodiment described herein relates to a method of using a CBₓ modulator as a K_{ATP} channel modulator for the treatment of a medical condition by administering a CBₓ modulator.

Another embodiment described herein relates to a method of treating a medical condition by administering to a subject in need thereof a CBₓ modulator.

Another embodiment described herein relates to a method of modulating a K_{ATP} channel in a mammal by administering a CBₓ modulator.

A further embodiment also includes a method of increasing insulin secretion in mammals by administering to a mammal in need thereof a CBₓ modulator as a K_{ATP} channel modulator.

An additional embodiment described herein is a method for modulating the biological activity of a cannabinoid receptor by contacting a CBₓ receptor modulator with the cannabinoid receptor wherein the CBₓ receptor modulator binds to the CBₓ receptor and a K_{ATP} channel is modulated.

Another embodiment is a complex comprising a cannabinoid receptor and a CBₓ receptor modulator wherein the CBₓ receptor modulator and the cannabinoid receptor are in contact to form the complex whereby a K_{ATP} channel is modulated.

Another embodiment described herein is a method of identifying compounds that modulate a cannabinoid receptor.

Other embodiments, objects, features and advantages will be set forth in the detailed description of the embodiments that follows, and in part will be apparent from the description, or may be learned by practice, of the claimed invention. These objects and advantages will be realized and attained by the processes and compositions particularly pointed out in the written description and claims hereof. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### DESCRIPTION

While the present invention is capable of being embodied in various forms, the description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. The various embodiments disclosed herein may be combined with other embodiments for the creation and description of yet additional embodiments.

One embodiment described herein relates to a method of using a CBₓ modulator as K_{ATP} channel modulator for the treatment of a medical condition in a mammal by administering a pharmaceutically effective amount of a CBₓ modulator. In a further embodiment, the CBₓ modulator is selected from the group consisting of: CBₓ agonists, CB₂ agonists, CB₂ partial agonists, CB₂ antagonists, CB₂ inverse agonists, compounds having both CBₓ agonist and CB₂ agonist properties, and mixtures thereof. In an additional embodiment, the medical condition to be treated is selected from the group consisting of obesity, diabetes mellitus, metabolic syndrome, syndrome X, insulinoma, familial hyperinsulemic hypoglycemia, male pattern baldness, detrusor hyperreactivity, asthma, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain, idiopathic pain and impotence.

In an additional embodiment, the metabolic syndrome or syndrome X to be treated includes disorders or diseases selected from the group consisting of hypertension, insulin resistance, glucose intolerance and dyslipoproteinaemia.

In a further embodiment, the CBₓ modulator is a modulator of at least one channel selected from the group consisting of Kir6.2/SUR1K_{ATP}, Kir6.2/SUR2B K_{ATP}, Kir6.1/SUR2B K_{ATP}, and Kir6.2/SUR2A K_{ATP}. While in another embodiment, the method of using a CBₓ modulator as a K_{ATP} channel modulator for the treatment of a medical condition further comprises a K_{ATP} channel modulator which is a K_{ATP} channel opener.

An additional embodiment includes a method of treating a medical condition in a mammal by administering to a subject in need thereof a pharmaceutically effective amount of a CBₓ modulator. In a further embodiment the CBₓ modulator is selected from the group consisting of: CBₓ agonists, CB₂ agonists, CB₂ partial agonists, CB₂ antagonists, CB₂ inverse agonists, compounds having both CBₓ agonist and CB₂ agonist properties, and mixtures thereof. In an additional embodiment the medical condition is selected from the group consisting of obesity, diabetes mellitus, metabolic syndrome, syndrome X, insulinoma, familial hyperinsulemic hypoglycemia, male pattern baldness, detrusor hyperreactivity, asthma, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain, idiopathic pain and impotence.

An additional embodiment includes a method of modulating a K_{ATP} channel in a mammal by administering to a mammal in need thereof a CBₓ modulator. In a further embodiment, the CBₓ modulator is selected from the group consisting of CB₁ agonists, CB₂ agonists, CB₂ partial agonists, CB₂ antagonists, CB₂ inverse agonists, compounds having both CBₓ agonist and CB₂ agonist properties, and mixtures thereof.

A further embodiment includes a method of increasing insulin secretion in mammals by administering to a mammal in need thereof a pharmaceutically effective amount of a CBₓ modulator as a K_{ATP} channel modulator.

An embodiment includes a method for modulating the biological activity of a cannabinoid receptor by contacting a CBₓ receptor modulator with the cannabinoid receptor, wherein the CBₓ receptor modulator binds to the CBₓ receptor and a K_{ATP} channel is modulated.

A further embodiment includes a complex comprising a cannabinoid receptor and a CBₓ receptor modulator wherein the CBₓ receptor modulator and the cannabinoid receptor are in contact to form the complex whereby a K_{ATP} channel is modulated.

An additional embodiment includes a method of identifying compounds that modulate a cannabinoid receptor by combining a test compound suspected of being a modulator of a cannabinoid receptor with COS-cells transiently expressing either hamster SUR1 in the presence of [³H] glibenclamide or rat SUR2A in the presence of [³H]P1075 and assessing the binding affinity of the test compound at sulfonylurea and K_{ATP} channel opening sites.

The terms “treat”, “treating” and “treatment” are to be broadly understood as referring to any response to, or anticipation of, a medical condition in a mammal, particularly a human, and includes but is not limited to:
(i) preventing the medical condition from occurring in a subject, which may or may not be predisposed to the condition, but has not yet been diagnosed with the condition and, accordingly, the treatment constitutes prophylactic treatment for the medical condition;
(ii) inhibiting the medical condition, i.e., arresting, slowing or delaying the on-set, development or progression of the medical condition; or
(iii) relieving the medical condition, i.e., causing regression of the medical condition.

The disclosure of U.S. patent application Ser. No. ______, filed on Apr. _{—}, 2007 which also claims the benefit of U.S. Provisional Application Nos. 60/745,757 and 60/745,760 (both filed on Apr. 27, 2006) is hereby incorporated by reference in its entirety to the extent permitted by law.

Suitable K_{ATP} channel modulators are compounds which have effects as full or partial openers at the Kir6.2/SUR1 K_{ATP} channel and/or the Kir6.2/SUR2 K_{ATP} channel. Effective compounds include those which exhibit an IC₅₀ value [μmol] of less than 50 in a test for the affinity of the compounds in binding to the sulfonylurea (SUR) and potassium channel opener site (KCO) of rat and/or human isoforms of SUR1 and/or SUR2—e.g. the test model provided below. Suitable compounds also include those with an effect as full or partial openers at the Kir6.2/SUR1 K_{ATP} channel, such as selective openers at the Kir6.2/SUR1 K_{ATP} channel. A compound with an effect as full or partial opener at the Kir6.2/SUR1 K_{ATP} channel is understood to be selective if its IC₅₀ value at the Kir6.2/SUR1 K_{ATP} channel, as measured in the aforementioned binding test, is less than half, more preferred only a quarter, of the IC₅₀ value of that same compound at the Kir6.2/SUR2 K_{ATP} channel.

It has surprisingly been found that CBₓ modulators from distinct structural classes act as potent and SUR1/SUR2 selective K_{ATP} Kir6.2 channel modulators. CBₓ modulators described herein bind to the CB₁ and CB₂ receptors to form a complex with the receptor and are CBₓ agonists; CB₂ agonists; CB₂ partial agonists; CB₂ antagonists; CB₂ inverse agonists; compounds having both CBₓ agonist and CB₂ agonist properties; and mixtures thereof. Suitable K_{ATP} channel modulators are SUR1/K_{ATP} Kir6.2 channel modulators, SUR1/K_{ATP} Kir6.2 channel modulators or partial SUR1/K_{ATP} Kir6.2 channel modulators. Suitable CBₓ modulators are compounds which have effects as modulators at the Kir6.2/SUR1 K_{ATP} channel, the Kir6.2/SUR2B K_{ATP} channel, the Kir6.1/SUR2B K_{ATP} channel, and/or the Kir6.2/SUR2A K_{ATP} channel.

In one embodiment, the K_{ATP} channel modulator is a K_{ATP} channel opener.

CBₓ modulators which are suitable for use as a K_{ATP} channel modulator include, but are not limited to the group consisting of: 3-(1,1-dimethyl-butyl)-6,6,9-trimethyl-6a,7,10,10a-tetrahydro-6H-benzo[c]chromene; N-Adamantyl-4-pentyl-5-phenyl-thiazole-2-carboxamide; N-{1,3,3-Trimethyl-endo-(1S)-bicyclo[2.2.1]hept-2-yl}-1-[1-(4-methyl)-benzyl-5-(4-chloro-3-methyl-phenyl)-1H-pyrazol-3-carboxamide; (2-Iodo-5-nitro-phenyl)-[1-(1-methyl-piperidin-2-ylmethyl)-1H-indol-3-yl]-methanone; {4-[4-(1,1-Dimethyl-heptyl)-2,6-dimethoxy-phenyl]-6,6-dimethyl-bicyclo[3.1.1]hept-2-en-2-yl}-methanol; 3-(1,1-Dimethyl-heptyl)-9-hydroxymethyl-6,6-dimethyl-6a,7,10,10a-tetrahydro-6H-enzo[c]chromen-1-ol; Icosa-5,8,11,14-tetraenoic acid 2-hydroxy-1-hydroxymethyl-ethyl ester; 1-Aziridin-1-yl-henicosa-6,9,12,15-tetraen-2-one; Noladineether; 4,4,4-Trifluoro-butane-1-sulfinic acid 3-(2-hydroxymethyl-indan-4-yloxy)-phenyl-ester, compound with form aldehyde; 7-Methoxy-2-oxo-8-pentyloxy-1,2-dihydro-quinoline-3-carboxylic acid (benzo[1,3]dioxol-5-ylmethyl)-amide; N-(1-{4-[4-Chloro-2-(2-fluoro-benzenesulfonyl)-benzenesulfonyl]-phenyl}-ethyl)-methanesulfonamide; [6-Iodo-2-methyl-1-(2-morpholin-4-yl-ethyl)-2,3-dihydro-1H-indol-3-yl]-(4-methoxy-phenyl)-methanone; 1-(4-Chloro-phenyl)-2-(2-chloro-phenyl)-5-ethyl-1H-imidazole-4-carboxylic acid piperidin-1-ylamide; (2-Methyl-1-propyl-2,3-dihydro-1H-indol-3-yl)-naphthalen-1-yl-methanone; 5-(1,1-Dimethyl-heptyl)-2-[5-hydroxy-2-(3-hydroxy-propyl)-cyclohexyl]-phenol; (2-Methyl-3-morpholin-4-ylmethyl-3,4-dihydro-5-oxa-2a-azacenaphthylen-1-yl)-naphthalen-1-yl-methanone; 5-(4-Chloro-phenyl)-1-(2,4-dichloro-phenyl)-4-methyl-1H-pyrazole-3-carboxylic acid piperidin-1-ylamide; 5-(4-Bromo-phenyl)-1-(2,4-dichloro-phenyl)-4-ethyl-1H-pyrazole-3-carboxylic acid piperidin-1-ylamide; 1-[Bis-(4-chloro-phenyl)-methyl]-3-[(3,5-difluoro-phenyl)-methanesulfonyl-methylene]-azetidine; 4-Chloro-N-{[3-(4-chloro-phenyl)-4-phenyl-4,5-dihydro-pyrazol-1-yl]-methylamino-methylene}-benzenesulfonamide; N-{Amino-[3-(4-chloro-phenyl)-4-phenyl-4,5-dihydro-pyrazol-1-yl]-methylene}-4-chloro-benzenesulfonamide; N-{[3-(4-Chloro-phenyl)-4-pyridin-3-yl-4,5-dihydro-pyrazol-1-yl]-methylamino-methylene}-4-trifluoromethyl-benzenesulfonamide; 4-Chloro-N-{[3-(4-chloro-phenyl)-4-pyridin-3-yl-4,5-dihydro-pyrazol-1-yl]-methylamino-methylene}-benzenesulfonamide; 4-Chloro-N-{[3-(4-chloro-phenyl)-4-(3-fluoro-phenyl)-4,5-dihydro-pyrazol-1-yl]-methoxyamino-methylene}-benzenesulfonamide; Morpholine-4-sulfonic acid [3-(4-chloro-phenyl)-4-phenyl-4,5-dihydro-pyrazol-1-yl]-methylamino-methyleneamide; N-{[3-(4-Chloro-phenyl)-4-(3-fluoro-phenyl)-4,5-dihydro-pyrazol-1-yl]-methylaminb-methylene}-N,N-dimethyl-sulfonamide; Azepane-1-sulfonic acid [3-(4-chloro-phenyl)-4-phenyl-4,5-dihydro-pyrazol-1-yl]-methylamino-methyleneamide; 4-Chloro-N-{[3-(4-chloro-phenyl)-4-phenyl-4,5-dihydro-pyrazol-1-yl]-[(1-methyl-pyrrolidin-3-ylmethyl)-amino]-methylene}-benzenesulfonamide; 1-(4-Chloro-phenyl)-5-phenyl-4,5-dihydro-1H-pyrazole-3-carboxamidine; N-{[3-(4-Chloro-phenyl)-4-phenyl-4,5-dihydro-pyrazol-1-yl]-methylamino-methylene}-4-trifluoromethyl-benzene-sulfonamide; Piperidine-1-sulfonic acid [3-(4-chloro-phenyl)-4-phenyl-4,5-dihydro-pyrazol-1-yl]-methylamino-methyleneamide; Piperidine-1-sulfonic acid [3-(4-chloro-phenyl)-4-phenyl-4,5-dihydro-pyrazol-1-yl]-(2-dimethylamino-ethylamino)-methyleneamide; N,N-Diethylamino-1-sulfonic acid [3-(4-chloro-phenyl)-4-phenyl-4,5-dihydro-pyrazol-1-yl]-methylsulfanyl-methyleneamide; 2-Amino-1-[3-(4-chloro-phenyl)-4-phenyl-4,5-dihydro-pyrazol-1-yl]-3-(3,4-dichloro-phenyl)-propan-1-one; Morpholine-4-sulfonic acid [3-(4-chloro-phenyl)-4-phenyl-4,5-dihydro-pyrazol-1-yl]-methylamino-methyleneamide; N,N-Dimethylamino-1-sulfonic acid [3-(4-chloro-phenyl)-4-phenyl-4,5-dihydro-pyrazol-1-yl]-(2-fluoro-ethylamino)-methyleneamide; Piperidine-1-sulfonic acid [3-(4-chloro-phenyl)-4-(3-fluoro-phenyl)-4,5-dihydro-pyrazol-1-yl]-methylamino-methyleneamide; 5-(4-Chloro-phenyl)-1-(2,4-dichloro-phenyl)-4,5-dihydro-1H-pyrazole-3-carboxylic acid piperidine-1-ylamide; 1-(4-Chloro-phenyl)-5-phenyl-4,5-dihydro-1H-pyrazole-3-carboxylic acid piperidin-1-ylamide; Piperidine-1-sulfonic acid [1-(4-chloro-phenyl)-5-phenyl-4,5-dihydro-1H-pyrazol-3-yl]-methylamino-methyleneamide; Morpholine-4-sulfonic acid [1-(2,4-dichloro-phenyl)-5-phenyl-4,5-dihydro-1H-pyrazol-3-yl]-methylamino-methyleneamide; 4-Chloro-N-[[3-(4-chloro-phenyl)-4-phenyl-4,5-dihydro-pyrazol-1-yl]-(2-fluoro-ethylamino)-methylene]-benzenesulfonamide; 4-Chloro-N-[[3-(4-chloro-phenyl)-4-phenyl-4,5-dihydro-pyrazol-1-yl]-(2-fluoro-ethylamino)-methylene]-benzenesulfonamide; N-{Amino-[3-(4-chloro-phenyl)-4-phenyl-4,5-dihydro-pyrazol-1-yl]-methylene}-4-chloro-benzenesulfonamide; 4-Chloro-N-[3-(4-chloro-phenyl)-4-phenyl-4,5-dihydro-pyrazole-1-carbonyl]-benzenesulfonamide; 4-Chloro-N-[[3-(4-chloro-phenyl)-4-phenyl-4,5-dihydro-pyrazol-1-yl]-(2-ethylamino-ethylamino)-methylene]-benzenesulfonamide; 4-Chloro-N-{[3-(4-chloro-phenyl)-4-phenyl-4, 5-dihydro-pyrazol-1-yl]-[(1-methyl-pyrrolidin-2-ylmethyl)-amino]-methylene}-benzenesulfonamide; 4-Chloro-N-[[3-(4-chloro-phenyl)-4-phenyl-4,5-dihydro-pyrazol-1-yl]-(4-pyrrolidin-1-yl-butylamino)-methylene]-benzenesulfonamide; 4-Chloro-N-{[3-(4-chloro-phenyl)-4-phenyl-4,5-dihydro-pyrazol-1-yl]-[(pyridin-3-ylmethyl)-amino]-methylene}-benzenesulfonamide; 1-[3-(4-Chloro-phenyl)-4-phenyl-4,5-dihydro-pyrazol-1-yl]-3-(1H-indol-2-yl)-2-methylamino-propan-1-one; 2-[3-(4-Chloro-phenyl)-4-phenyl-4,5-dihydro-pyrazol-1-yl]-5-ethyl-4,5-dihydro-oxazole; 4-Chloro-N-[[3-(4-chloro-phenyl)-4-phenyl-4,5-dihydro-pyrazol-1-yl]-(3-hydroxy-2,2-dimethyl-propylamino)-methylene]-benzenesulfonamide; N,N-Diethylamino-1-sulfonic acid [3-(4-chloro-phenyl)-4-hydroxy-4-phenyl-4,5-dihydro-pyrazol-1-yl]-methylamino-methyleneamide; 5-(4-Bromo-phenyl)-1-(2,4-dichloro-phenyl)-1H-pyrazole-3-carbonitrile; 8-Chloro-1-(2,4-dichloro-phenyl)-1,3a,4,5,6,10b-hexahydro-1,2-diaza-benzo[e]azulene-3-carboxylic acid piperidin-1-ylamide; 5-(4-Bromo-phenyl)-1-(2,4-dichloro-phenyl)-3-[2-(3,5-difluoro-phenyl)-2-methanesulfonyl-vinyl]-4-methyl-1H-pyrazole; Piperidine-1-carboxylic acid [5-(4-chloro-phenyl)-1-(2,4-dichloro-phenyl)-4-methyl-1H-pyrazol-3-yl]-amide; 1-(4-Chloro-phenyl)-2-(2,4-dichloro-phenyl)-5-ethylsulfanyl-1H-imidazole-4-carboxylic acid piperidin-1-ylamide; 2-(2,4-Dichloro-phenyl)-1-(4-trifluoromethyl-phenyl)-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid piperidin-1-ylamide; 1-(4-Chloro-phenyl)-2-(2,4-dichloro-phenyl)-1H -imidazole-4-carboxylic acid piperidin-1-yiamide; 1-(4-Chloro-phenyl)-2-(2,4-dichloro-phenyl)-5-ethyl-1H-imidazole-4-carboxylic acid piperidin-1-ylamide; 1-(4-Bromo-phenyl)-2-(2,4-dichloro-phenyl)-5-ethyl-1H-imidazole-4-carboxylic acid piperidin-1-ylamide; 1-(4-Bromo-phenyl)-5-chloro-2-(2,4-dichloro-phenyl)-1H-imidazole-4-carboxylic acid piperidin-1-ylamide; 1-(4-Bromo-phenyl)-2-(2,4-dichloro-phenyl)-5-ethyl-1H-imidazole-4-carboxylic acid cyclohexylamide; 1-(4-Bromo-phenyl)-2-(2,4-dichloro-phenyl)-5-ethyl-1H-imidazole-4-carboxylic acid pentylamide; 4-(4-Chloro-phenyl)-5-(2,4-dichloro-phenyl)-1-methyl-1H-imidazole-2-carboxylic acid cyclohexylamide; 4-(4-Chloro-phenyl)-5-(2,4-dichloro-phenyl)-3-methyl-1H-imidazole-2-carboxylic acid cyclohexylamide; 1-(5-Chloro-pyridin-2-yl)-2-(2,4-dichloro-phenyl)-5-ethyl-1H-imidazole-4-carboxylic acid piperidin-1-ylamide; 1-(4-Chloro-phenyl)-2-(2,4-dichloro-phenyl)-5-methyl-1H-imidazole-4-carboxylic acid (4-hydroxy-cyclo-hexyl)-amide; 1-(4-Chloro-phenyl)-2-(2,4-dichloro-phenyl)-5-methyl-1H-imidazole-4-carboxylic acid azepan-1-ylamide; 2-(2,4-Dichloro-phenyl)-5-ethyl-1-phenyl-1H-imidazole-4-carboxylic acid piperidin-1-ylamide; 2-(1,5-Dimethyl-1H-pyrrol-2-yl)-5-ethyl-1-phenyl-1H-imidazole-4-carboxylic acid cyclohexylamide; 1-(4-Chloro-phenyl)-5-ethyl-2-(3-methyl-pyridin-2-yl)-1H-imidazole-4-carboxylic acid piperidin-1-ylamide; 1-(4-Chloro-phenyl)-2-(2,4-dichloro-phenyl)-5-ethyl-1H-imidazole-4-carboxylic acid piperidin-1-ylamide; 1-(4-Bromo-phenyl)-2-(2,4-dichloro-phenyl)-5-ethyl-1H-imidazole-4-carboxylic acid piperidin-1-ylamide; 1-(4-Bromo-phenyl)-5-chloro-2-(2,4-dichloro-phenyl)-1H-imidazole-4-carboxylic acid piperidin-1-ylamide; 1-(4-Bromo-phenyl)-2-(2,4-dichloro-phenyl)-5-ethyl-1H-imidazole-4-carboxylic acid cyclohexylamide; 1-(4-Bromo-phenyl)-2-(2,4-dichloro-phenyl)-5-ethyl-1H-imidazole-4-carboxylic acid pentylamide; 4-(4-Chloro-phenyl)-5-(2,4-dichloro-phenyl)-1-methyl-1H-imidazole-2-carboxylic acid cyclohexylamide; 4-(4-Chloro-phenyl)-5-(2,4-dichloro-phenyl)-3-methyl-1H-imidazole-2-carboxylic acid cyclohexylamide; 1-(5-Chloro-pyridin-2-yl)-2-(2,4-dichloro-phenyl)-5-ethyl-1H-imidazole-4-carboxylic acid piperidin-1-ylamide; 1-(4-Chloro-phenyl)-2-(2,4-dichloro-phenyl)-5-methyl-1H-imidazole-4-carboxylic acid (4-hydroxy-cyclo-hexyl)-amide; 1-(4-Chloro-phenyl)-2-(2,4-dichloro-phenyl)-5-methyl-1H-imidazole-4-carboxylic acid azepan-1-ylamide; 2-(2,4-Dichloro-phenyl)-5-ethyl-1-phenyl-1H-imidazole-4-carboxylic acid piperidin-1-ylamide; 2-(1,5-Dimethyl-1H-pyrrol-2-yl)-5-ethyl-1-phenyl-1H-imidazole-4-carboxylic acid cyclohexylamide; 1-(4-Chloro-phenyl)-5-ethyl-2-(3-methyl-pyridin-2-yl)-1H-imidazole-4-carboxylic acid piperidin-1-ylamide; 1-(4-Chloro-phenyl)-5-ethyl-2-(3-methyl-pyridin-2-yl)-1H-imidazole-4-carboxylic acid cyclohexylamide; 1-(4-Chloro-phenyl)-2-(2,4-dichloro-phenyl)-5-methyl-1H-imidazole-4-carboxylic acid (4-trifluoromethyl-phenyl)-amide; 2-(2,4-Dichloro-phenyl)-5-methyl-1-pyridin-2-yl-1H-imidazole-4-carboxylic acid piperidin-1-ylamide; 1-(4-Chloro-phenyl)-2-(2,4-dichloro-phenyl)-5-fluoromethyl-1H-imidazole-4-carboxylic acid piperidin-1-ylamide; 1-(4-Chloro-phenyl)-2-(2,4-dichloro-phenyl)-5-hydroxymethyl-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid cyclohexylamide; 1-(4-Chloro-phenyl)-2-(2,4-dichloro-phenyl)-5-methanesulfonyl-1H-imidazole-4-carboxylic acid piperidin-1-yiamide; 1-(4-Chloro-phenyl)-2-(2,4-dichloro-phenyl)-5-methanesulfinyl-1H-imidazole-4-carboxylic acid piperidin-1-ylamide; 5-(4-Chloro-phenyl)-4-(2,5-dichloro-phenyl)-1-methyl-1H-imidazole-2-carboxylic acid piperidin-1-ylamide; 2-(2-Chloro-phenyl)-1-(5-chloro-pyridin-2-yl)-5-ethyl-1H-imidazole-4-carboxylic acid piperidin-1-ylamide; 1-(4-Chloro-phenyl)-2-(2,4-dichloro-phenyl)-5-(2,2,2-trifluoro-ethyl)-1H-imidazole-4-carboxylic acid piperidin-1-ylamide; N-[1-(4-Chloro-phenyl)-2-(2,4-dichloro-phenyl)-5-methyl-1H-imidazol-4-yl]-benzamide; 1-(4-Chloro-phenyl)-2-(2,4-dichloro-phenyl)-5-pyrrolidin-1-ylmethyl-1H-imidazole-4-carboxylic acid piperidin-1-ylamide; 2-[1-(4-Chloro-phenyl)-2-(2,4-dichloro-phenyl)-5-methyl-1H-imidazol-4-yl]-hexan-2-ol; 1-(4-Chloro-phenyl)-2-(2,4-dichloro-phenyl)-5-methyl-4-pentyl-1H-imidazole; 2,5-Dimethyl-1-phenyl-1H-imidazole-4-carboxylic acid adamantan-2-ylamide; 1-(4-Chloro-phenyl)-2-(2-chloro-phenyl)-5-methylsulfanyl-1H-imidazole-4-carboxylic acid piperidin-1-ylamide; 2-(2-Chloro-phenyl)-1-(4-trifluoromethyl-phenyl)-1H-imidazole-4-carboxylic acid piperidin-1-ylamide; 5-(4-Chloro-phenyl)-4-(2,4-dichloro-phenyl)-thiazole-2-carboxylic acid piperidin-1-ylamide; 5-(4-Chloro-phenyl)-1-(2,4-dichloro-phenyl)-1H-[1,2,4]triazole-3-carboxylic acid pyrrolidin-1-ylamide; 1-(4-Chloro-phenyl)-5-(2,4-dichloro-phenyl)-1H-[1,2,4]triazole-3-carboxylic acid piperidin-1-yl-amide; 5-Pentyl-4-phenyl-thiazole-2-carboxylic acid (hexahydro-2,5-methano-pentalen-3a-yl)-amide; 4-Pentyl-5-phenyl-thiazole-2-carboxylic acid (hexahydro-2,5-methano-pentalen-3a-yl)-amide; 1-{(4-Chloro-benzene-sulfonylimino)-[3-(4-chloro-phenyl)-4-phenyl-4,5-dihydro-pyrazol-1-yl]-methyl}-piperidine-4-carboxylic acid amide; 4-Chloro-N-{[3-(4-chloro-phenyl)-4-phenyl-4,5-dihydro-pyrazol-1-yl]-[2-(2-oxo-pyrrolidin-1-yl)-ethylamino]-methylene}-benzenesulfonamide; 4-Chloro-N-[[3-(4-chloro-phenyl)-4-phenyl-4,5-dihydro-pyrazol-1-yl]-(2-cyano-ethylamino)-methylene]-benzene-sulfonamide; 4-Chloro-N-[[3-(4-chloro-phenyl)-4-phenyl-4,5-dihydro-pyrazol-1-yl]-(methoxy-methyl-amino)-methylene]-benzenesulfonamide; 4-Chloro-N-{[3-(4-chloro-phenyl)-4-phenyl-4,5-dihydro-pyrazol-1-yl]-[(piperidin-4-ylmethyl)-amino]-methylene}-benzenesulfonamide; 4-Chloro-N-[[3-(4-chloro-phenyl)-4-phenyl-4,5-dihydro-pyrazol-1-yl]-(piperidin-4-ylamino)-methylene]-benzenesulfonamide; and Morpholine-4-sulfonic acid [3-(4-chloro-phenyl)-4-phenyl-4,5-dihydro-pyrazol-1-yl]-(cyclopropylmethyl-amino)-methyleneamide.

In another embodiment, CBₓ modulators which are suitable for use as a K_{ATP} channel modulator include those selected from the group consisting of: 3-(1,1-dimethyl-butyl)-6, 6,9-trimethyl-6a,7,10,10a-tetrahydro-6H-benzo[c]chromene; N-Adamantyl-4-pentyl-5-phenyl-thiazole-2-carboxamide; N-{1,3,3-Trimethyl-endo-(1S)-bicyclo[2.2.1]hept-2-yl}-1-[1-(4-methyl)-benzyl-5-(4-chloro-3-methyl-phenyl)-1H-pyrazol-3-carboxamide; (2-Iodo-5-nitro-phenyl)-[1-(1-methyl-piperidin-2-ylmethyl)-1H-indol-3-yl]-methanone; {4-[4-(1,1-Dimethyl-heptyl)-2,6-dimethoxy-phenyl]-6,6-dimethyl-bicyclo[3.1.1]hept-2-en-2-yl}-methanol; 3-(1,1-Dimethyl-heptyl)-9-hydroxymethyl-6,6-dimethyl-6a,7,10,10a-tetrahydro-6H-enzo[c]chromen-1-ol; Icosa-5,8,11,14-tetraenoic acid 2-hydroxy-1-hydroxymethyl-ethyl ester; 1-Aziridin-1-yl-henicosa-6,9,12,15-tetraen-2-one; Noladineether; 4,4,4-Trifluoro-butane-1-sulfinic acid 3-(2-hydroxymethyl-indan-4-yloxy)-phenyl ester, compound with form aldehyde; 7-Methoxy-2-oxo-8-pentyloxy-1,2-dihydro-quinoline-3-carboxylic acid (benzo[1,3]dioxol-5-ylmethyl)-amide; N-(1-{4-[4-Chloro-2-(2-fluoro-benzenesulfonyl)-benzenesulfonyl]-phenyl}-ethyl)-methanesulfonamide; [6-Iodo-2-methyl-1-(2-morpholin-4-yl-ethyl)-2,3-dihydro-1H-indol-3-yl]-(4-methoxy-phenyl)-methanone; 1-(4-Chloro-phenyl)-2-(2-chloro-phenyl)-5-ethyl-1H-imidazole-4-carboxylic acid piperidin-1-ylamide; (2-Methyl-1-propyl-2,3-dihydro-1H-indol-3-yl)-naphthalen-1-yl-methanone; 5-(1,1-Dimethyl-heptyl)-2-[5-hydroxy-2-(3-hydroxy-propyl)-cyclohexyl]-phenol; (2-Methyl-3-morpholin-4-ylmethyl-3,4-dihydro-5-oxa-2a-azacenaphthylen-1-yl)-naphthalen-1-yl-methanone; 5-(4-Chloro-phenyl)-1-(2,4-dichloro-phenyl)-4-methyl-1H-pyrazole-3-carboxylic acid piperidin-1-ylamide; 5-(4-Bromo-phenyl)-1-(2,4-dichloro-phenyl)-4-ethyl-1H-pyrazole-3-carboxylic acid piperidin-1-ylamide; 1-[Bis-(4-chloro-phenyl)-methyl]-3-[(3,5-difluoro-phenyl)-methanesulfonyl-methylene]-azetidine.

In another embodiment, CBₓ modulators which are suitable for use as a K_{ATP} channel modulator include those selected from the group consisting of: 4-Chloro-N-{[3-(4-chloro-phenyl)-4-phenyl-4,5-dihydro-pyrazol-1-yl]-methylamino-methylene}-benzenesulfonamide; N-{Amino-[3-(4-chloro-phenyl)-4-phenyl-4,5-dihydro-pyrazol-1-yl]-methylene}-4-chloro-benzenesulfonamide; 4-Chloro-N-{[3-(4-chloro-phenyl)-4-pyridin-3-yl-4,5-dihydro-pyrazol-1-yl]-methylamino-methylene}-benzenesulfonamide; 4-Chloro-N-{[3-(4-chloro-phenyl)-4-(3-fluoro-phenyl)-4,5-dihydro-pyrazol-1-yl]-methoxyamino-methylene}-benzenesulfonamide; N-{[3-(4-Chloro-phenyl)-4-(3-fluoro-phenyl)-4,5-dihydro-pyrazol-1-yl]-methylamino-methylene}-N,N-dimethyl-sulfonamide; 5-(4-Chloro-phenyl)-1-(2,4-dichloro-phenyl)-4,5-dihydro-1H-pyrazole-3-carboxylic acid piperidine-1-ylamide; Morpholine-4-sulfonic acid [1-(2,4-dichloro-phenyl)-5-phenyl-4,5-dihydro-1H-pyrazol-3-yl]-methylamino-methyleneamide; N-{Amino-[3-(4-chloro-phenyl)-4-phenyl-4,5-dihydro-pyrazol-1-yl]-methylene}-4-chloro-benzenesulfonamide; 4-Chloro-N-[[3-(4-chloro-phenyl)-4-phenyl-4,5-dihydro-pyrazol-1-yl]-(2-ethylamino-ethylamino)-methylene]-benzenesulfonamide; 4-Chloro-N-{[3-(4-chloro-phenyl)-4-phenyl-4,5-dihydro-pyrazol-1-yl]-[(1-methyl-pyrrolidin-2-ylmethyl)-amino]-methylene}-benzenesulfonamide; 4-Chloro-N-{[3-(4-chloro-phenyl)-4-phenyl-4,5-dihydro-pyrazol-1-yl]-[(pyridin-3-ylmethyl)-amino]-methylene}-benzenesulfonamide; 1-(4-Chloro-phenyl)-2-(2,4-dichloro-phenyl)-5-ethylsulfanyl-1H-imidazole-4-carboxylic acid piperidin-1-ylamide; 2-(2,4-Dichloro-phenyl)-1-(4-trifluoromethyl-phenyl)-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid piperidin-1-ylamide; 1-(4-Chloro-phenyl)-2-(2,4-dichloro-phenyl)-5-ethyl-1H-imidazole-4-carboxylic acid piperidin-1-ylamide; 1-(4-Bromo-phenyl)-2-(2,4-dichloro-phenyl)-5-ethyl-1H-imidazole-4-carboxylic acid piperidin-1-ylamide; 1-(4-Bromo-phenyl)-5-chloro-2-(2,4-dichloro-phenyl)-1H-imidazole-4-carboxylic acid piperidin-1-ylamide; 1-(4-Bromo-phenyl)-2-(2,4-dichloro-phenyl)-5-ethyl-1H-imidazole-4-carboxylic acid pentylamide; 1-(4-Chloro-phenyl)-2-(2,4-dichloro-phenyl)-5-methyl-1H-imidazole-4-carboxylic acid azepan-1-ylamide; 1-(4-Chloro-phenyl)-2-(2,4-dichloro-phenyl)-5-fluoromethyl-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid cyclohexylamide; N-[1-(4-Chloro-phenyl)-2-(2,4-dichloro-phenyl)-5-methyl-1H-imidazol-4-yl]-benzamide; 2-[1-(4-Chloro-phenyl)-2-(2,4-dichloro-phenyl)-5-methyl-1H-imidazol-4-yl]-hexan-2-ol; 1-(4-Chloro-phenyl)-2-(2,4-dichloro-phenyl)-5-methyl-4-pentyl-1H-imidazole; 1-(4-Chloro-phenyl)-2-(2-chloro-phenyl)-5-methylsulfanyl-1H-imidazole-4-carboxylic acid piperidin-1-ylamide; 2-(2-Chloro-phenyl)-1-(4-trifluoromethyl-phenyl)-1H-imidazole-4-carboxylic acid piperidin-1-ylamide; 5-(4-Chloro-phenyl)-4-(2,4-dichloro-phenyl)-thiazole-2-carboxylic acid piperidin-1-ylamide; 1-(4-Chloro-phenyl)-5-(2,4-dichloro-phenyl)-1H-[1,2,4]triazole-3-carboxylic acid piperidin-1-yl-amide; 1-{(4-Chloro-benzene-sulfonylimino)-[3-(4-chloro-phenyl)-4-phenyl-4,5-dihydro-pyrazol-1-yl]-methyl}-piperidine-4-carboxylic acid amide; 4-Chloro-N-{[3-(4-chloro-phenyl)-4-phenyl-4,5-dihydro-pyrazol-1-yl]-[2-(2-oxo-pyrrolidin-1-yl)-ethylamino]-methylene}-benzenesulfonamide; 4-Chloro-N-[[3-(4-chloro-phenyl)-4-phenyl-4,5-dihydro-pyrazol-1-yl]-(2-cyano-ethylamino)-methylene]-benzene-sulfonamide; 4-Chloro-N-[[3-(4-chloro-phenyl)-4-phenyl-4,5-dihydro-pyrazol-1-yl]-(methoxy-methyl-amino)-methylene]-benzenesulfonamide; Morpholine-4-sulfonic acid [3-(4-chloro-phenyl)-4-phenyl-4,5-dihydro-pyrazol-1-yl]-(cyclopropylmethyl-amino)-methyleneamide and mixtures thereof.

CB₁ agonist or CB₂ agonists which are suitable for use as a K_{ATP} channel modulator include, but are not limited to the group consisting of: L759633; L759656; {4-[4-(1,1-dimethyl-heptyl)-2,6-dimethoxy-phenyl]-6,6-dimethyl-bicyclo-[3.1.1]hept-2-en-2-yl}-methanol (HU308); JWH015; (2-iodo-5-nitro-phenyl)-[1-(1-methyl-piperidin-2-ylmethyl)-1H -indol-3-yl]-methanone (AM-1241); 3-(1,1-dimethyl-butyl)-6,6,9-trimethyl-6a,7,10,10a-tetrahydro-6H-benzo[c]-chromene (JWH133); N-adamantyl-4-pentyl-5-phenyl-thiazole-2-carboxamide; 6,6,9-trimethyl-3-pentyl-6a,7,8,10a-tetrahydro-6H-benzo[c]chromen-1-ol; (bicyclo[2.2.1]hept-2-ylamino)-(5-pentyl-4-phenyl-thiazol-2-yl)-methane; 5-(1,1-dimethyl-heptyl)-2-[5-hydroxy-2-(3-hydroxy-propyl)-cyclohexyl]-phenol (CP-55,940); (2-methyl-3-morpholin-4-ylmethyl-3,4-dihydro-5-oxa-2a-aza-acenaphthylen-1-yl)-naphthalen-1-yl-methanone (WIN-55,212-2); ACEA; ACPA; N-adamantyl-4-pentyl-5-phenyl-thiazole-2-carboxamide; methanandamide; anandamide; 2-arachidonoyl glycerol; 2-icosa-5,8,11,14-tetraenyloxy-propane-1,3-diol (noladin ether); BAY 38-7271; SAB-378; BAY 59-3074; O-1057; GW-1000; PRS-211375; PRS-211359; PRS-211355; PRS-211096; PXS-2076; AM-577; GW-842166X; and mixtures thereof.

In one embodiment, the CB₂ agonist is a selective CB₂ agonist and is selected from the group consisting of: 3-(1,1-dimethyl-butyl)-6,6,9-trimethyl-6a,7-,10a-tetrahydro-6H-benzo[c]chromene (JWH133); L759633; L759656; {4-[4-(1,1-dimethyl-heptyl)-2,6-dimethoxy-phenyl]-6,6-dimethyl-bicyclo[3.1.1]hept-2-en-2-yl}-methanol (HU308); JWH015; (2-iodo-5-nitro-phenyl)-[1-(1-methyl-piperidin-2-ylmethyl)-1H-indol-3-yl]-methanone (AM-1241); and mixtures thereof.

CB₂ antagonist or CB₂ inverse agonists which are suitable for use as K_{ATP} channel modulator are selected from the group consisting of: (1) compounds described in documents WO01/0588869, PCT/EP2006/060009, WO2004/014825; EP1142877; US2002/0072529; WO02/062750; U.S. Pat. No. 6,509,352; and (2) compounds selected from the group consisting of: 1-[1-benzyl-5-(4-chloro-3-methyl-phenyl)-1H-pyrazol-3-yl]-ethanonyl-1, 3,3-trimethyl-bi-cyclo[2.2.1]hept-2-ylamine (SR-144528), JTE-907, AM630, and mixtures thereof; and (3) mixtures of compounds selected from (1), (2) and (1) and/or (2).

Compounds having both CB₁ agonist and CB₂ agonist properties, and suitable for use as a K_{ATP} channel modulator include 2-icosa-5,8,11,14-tetraenyloxy-propane-1,3-diol (noladin ether).

In one embodiment, the CBₓ modulator is selected from the group consisting of: 6,6,9-trimethyl-3-pentyl-6a,7,8,10a-tetrahydro-6H-benzo[c]chromen-1-ol; (bicyclo[2.2.1]hept-2-ylamino)-(5-pentyl-4-phenyl-thiazol-2-yl)-methane; 3-(1,1-dimethyl-butyl)-6,6,9-trimethyl-6a,7,10,10a-tetrahydro-6H-benzo[c]chromene; N-adamantyl-4-pentyl-5-phenyl-thiazole-2-carboxamide; SR144528; (2-iodo-5-nitro-phenyl)-[1-(1-methyl-piperidin-2-ylmethyl)-1H-indol-3-yl]-methanone; {4-[4-(1,1-Dimethyl-heptyl)-2,6-dimethoxy-phenyl]-6,6-dimethyl-bicyclo[3.1.1]hept-2-en-2-yl}-methanol; and mixtures thereof.

A set of representative CBₓ modulators with their affinities for the K_{ATP} SUR1 and SUR2 regulatory subunits (hamster), respectively are depicted in Table 1. (2-Chloro-4-methyl-7,7-dioxo-4,7-dihydro-1,7lambda*6*-dithia-4,6-diaza-inden-5-yl)-(1-methyl-cyclopropyl)-amine and 7-Chloro-3-methyl-2H-benzo[1,2,4]thiadiazine 1,1-dioxide serve as K_{ATP}/SUR1 active reference compounds. For comparative purposes, Table 1 also lists the CB₁ and/or CB₂ activities of various compounds to show that their activity as CB₁ agonists, CB₂ agonists, CB₂ partial agonists, CB₂ antagonists, CB₂ inverse agonists and compounds having both CB₁ agonist and CB₂ agonist properties.

Description of the Pharmacological Test Methods

1. In Vitro Binding Affinity of the Test Compounds to Rodent K_{ATP} Channels

Competitive binding experiments were performed to characterize the affinity of the test compounds for the binding sites for sulfonylureas and K_{ATP} channel openers (KCOS) on hamster SUR1. To assess the affinity for the sulfonylurea site, membranes from COS-cells transiently expressing hamster SUR1 were incubated in the presence of [³H]glibenclamide with increasing concentrations of test compounds. The affinity for binding to the KCO site was assessed by incubations in the additional presence of 100 μM MgATP (see Schwanstecher M., et al. Naunyn-Schmiedeberg's Arch. Pharmacol. 343 (1991) 83-89 and Schwanstecher M. et al., EMBO J. 17 (1998) 5529-5535 (Schwanstecher et al., 1998)). For each test compound four displacement curves were measured (+/−MgATP from the human and hamster isoform). Per curve 9-15 distinct concentrations were tested covering the relevant range. All measurements were repeated at least 5 times in independent experiments.

Similar to SUR1 (see above) competitive binding experiments were performed to characterize the affinity of the test compounds for the binding sites for sulfonylureas and KCOs on rat SUR2A. The affinity for the KCO site on SUR2A was assessed by displacement of [³H]P1075 (see Schwanstecher et al., 1998; Dörschner H. et al. Mol. Pharmacol. 55 (1999) 1060-1066 (Dörschner et al., 1999)). The affinity of [³H]glibenclamide for the human SUR2 isoforms, however, is too weak to allow direct detection of binding using filtration assays. Therefore, two strategies can be used to detect binding to the sulfonylurea site on SUR2A. First, binding can be detected indirectly through allosteric displacement of [³H]P1075 (Dörschner et al., 1999). Second, a mutated SUR2A (SUR2A_{Y1205S}, see above) with increased affinity for [³H]glibenclamide allowing direct displacement of this tracer can be used. This second approach was chosen to enable discrimination between allosteric and competitive interaction with the KCO site and to make sure that binding of ligands which do not induce allosteric displacement are not overlooked.

Membranes from COS-cells transiently expressing rat SUR2A were incubated in the presence of the radioligands with increasing concentrations of test compounds as described above. The affinity for binding to the KCO site was assessed by incubations in the additional presence of 100 μM MgATP (Schwanstecher et al., 1991 and 1998). For each test compound four displacement curves were measured (displacement of [³H]P1075 from the rat isoform of the wild type receptor and displacement of [³H]glibenclamide from the rat isoform of SUR2A_{Y1205S}). Per curve 9-15 distinct concentrations were tested covering the relevant range. All measurements were repeated at least 5 times in independent experiments.

[³H]P1075 (specific activity 116 Ci mmol⁻¹) was purchased from Amersham Buchler (Braunschwëig, Germany). [³H]glibenclamide (specific activity 51 Ci mmol⁻¹) was obtained from NEN (Dreieich, Germany). Where needed, stock solutions were prepared in dimethylsulfoxide with a final solvent concentration in the media below 1%.

SUR- or Kir6.x isoforms were used either subcloned in the pcDNA (hamster SUR1, mouse Kir6.2) or pCMV vector (rat SUR2A, SUR2B).

Rodent SUR-isoforms and K_{ATP} channels were transiently expressed in COS-1 cells as described in Schwanstecher et al., 1998, Dörschner et al., 1999, Uhde I. et al. J Biol Chem 274 (1999) 28079-28082, Gross I. et al. Mol. Pharmacol. 56 (1999) 1370-1373 and Markworth E., Diabetes 49 (2000) 1413-1418. A mutated form of the SUR2 isoforms with the phenylalanine residue in position 1205 substituted with a serine (SUR2_{Y1205S}) was used to allow detection of binding to the sulfonylurea site of these isoforms by displacement of [³H]glibenclamide (Uhde I., Dissertation 2001). Briefly, COS-1 cells cultured in DMEM HG (10 mM glucose), supplemented with 10% fetal calf serum (FCS), were plated at a density of 5×10⁵ cells per dish (94 mm) and allowed to attach overnight. For transfection, the cells were incubated 4 hours in a Tris-buffered salt solution containing DNA (5-10 μg/ml) plus DEAE-dextran (1 mg/ml), 2 min in HEPES-buffered salt solution plus dimethylsulfoxide (10%) and 4 hours in DMEM-HG plus chloroquine (100 μM). Cells were then returned to DMEM-HG plus 10% FCS. Membranes were prepared 60-72 h post transfection as described (Schwanstecher M. et al., Br. J. Pharmacol. 106 (1992) 295-301 (Schwanstecher et al., 1992)). For binding experiments, resuspended membranes (final protein concentration 5-50 μg/ml) were incubated in “Tris-buffer” (50 mM, pH 7.4) containing either [³H]glibenclamide (final concentration 0.3 nM or 3 nM and nonspecific binding defined by 100 nM or 1 μM glibenclamide for SUR1 or SUR2_{Y1205S}-isoforms, respectively) or [³H]P1075 (final concentration 3 nM, nonspecific binding defined by 100 μM pinacidil) and increasing concentrations of the test compounds. The free Mg²⁺ concentration were kept close to 0.7 mM. ATP (0.1 mM) was added to incubation media to enable KCO (e.g. diazoxide, [³H]P1075) binding (see Schwanstecher et al., 1998). Incubations were carried out for 1 h at room temperature and were terminated by rapid filtration through Whatman GF/B filters.

The inhibition constant (Kᵢ value) of the test substances was calculated from the respective IC₅₀ value, and was stated as the negative logarithmic value thereof (pKᵢ).

2. In Vitro Binding Affinity of the Test Compounds to CB₁ Receptors

The affinity of the compounds of the present disclosure to form complexes with the cannabinoid CB₁ receptors can be determined using membrane preparations of Chinese hamster ovary (CHO) cells in which the human cannabinoid CB₁ receptor is stably transfected in conjunction with [³H]CP-55,940 as a radioligand. After incubation of a freshly prepared cell membrane preparation with the [³H]-ligand, with or without the addition of compounds described herein, separation of bound and free ligand is performed by filtration over glass-fiber filters. Radioactivity on the filter is measured by liquid scintillation counting.

3. In Vitro Binding Affinity of the Test Compounds to CB₂ Receptors

The affinity of the compounds of the present disclosure to form complexes with the cannabinoid CB₂ receptors can be determined using membrane preparations of Chinese hamster ovary (CHO) cells in which the human cannabinoid CB₂ receptor is stably transfected in conjunction with [³H]CP-55,940 as a radioligand. After incubation of a freshly prepared cell membrane preparation with the [³H]-ligand, with or without the addition of the compounds described herein, separation of bound and free ligand is performed by filtration over glass-fiber filters. Radioactivity on the filter is measured by liquid scintillation counting.

TABLE 1
CBₓ modulators with their affinities on the CB1 and/or CB2 receptor
affinities, (cloned human cannabinoid (CB₁ and CB₂ respectively) receptors expressed in
CHO cells according to the procedures described hereinabove), expressed as pKᵢ values.

| | | Affinity to | | | |
|---|---|---|---|---|---|
| compound/name | chemical name | CB1 | CB2 | SUR 1 | SUR 2 |
| 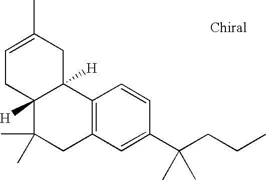 | 3-(1,1-dimethyl-butyl)- 6,6,9-trimethyl- 6a,7,10,10a-tetrahydro-6H- benzo[c]chromene (JW133) | 6.7 | 7.8 | 5.9 | 3.6 |
| 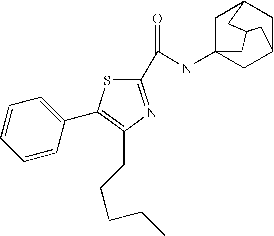 | N-Adamantyl-4-pentyl-5- phenyl-thiazole-2- carboxamide | 7.8 | 8.1 | 4.0 | 4.0 |
| 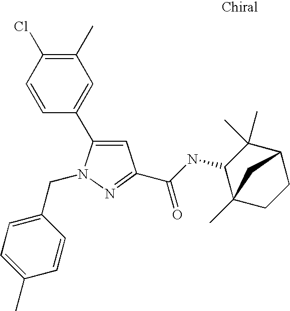 | N-{1,3,3-Trimethyl-endo- (1S)-bicyclo[2.2.1]hept-2- yl}-1-[1-(4-methyl)-benzyl- 5-(4-chloro-3-methyl- phenyl)-1H-pyrazol-3- carboxamide (SR 144528) | 6.2 | 7.7 | 6.9 | 4.8 |
| 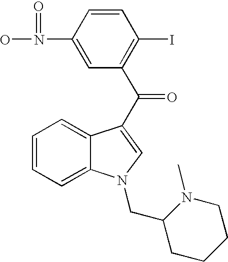 | (2-Iodo-5-nitro-phenyl)-[1- (1-methyl-piperidin-2- ylmethyl)-1H-indol-3-yl]- methanone (AM-1241) | 6.6 | 7.8 | 5.4 | 4.7 |
| 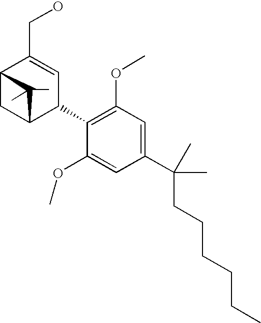 | {4-[4-(1,1-Dimethyl- heptyl)-2,6-dimethoxy- phenyl]-6,6-dimethyl- bicyclo[3.1.1]hept-2-en-2- yl}-methanol (HU308) | 6.0 | 7.8 | 6.4 | 3.6 |
| 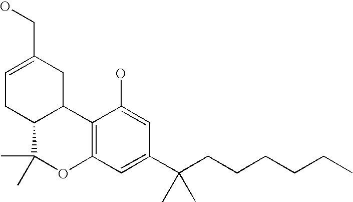 | 3-(1,1-Dimethyl-heptyl)-9- hydroxymethyl-6,6- dimethyl-6a,7,10,10a-tetra hydro-6H-enzo[c]chromen- 1-ol (HU-210) | 7.3 | n/a | 6.1 | 5.1 |
| 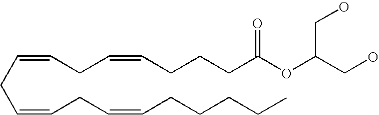 | Icosa-5,8,11,14-tetraenoic acid 2-hydroxy-1- hydroxymethyl-ethyl ester (2-AG) | 6.0 | 5.4 | 5.6 | 5.5 |
| 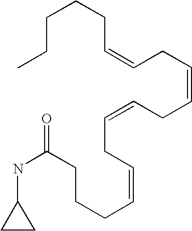 | 1-Aziridin-1-yl-henicosa- 6,9,12,15-tetraen-2-one (ACPA) | 7.7 | 7.1 | 3.9 | 4.7 |
| 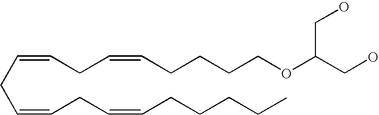 | Noladineether | 6.9 | 6.6 | 5.4 | 4.4 |
| 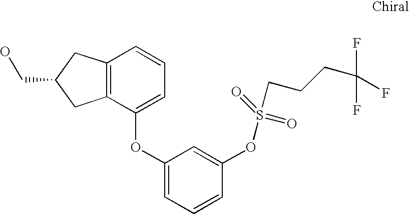 | 4,4,4-Trifluoro-butane-1- sulfinic acid 3-(2- hydroxymethyl-indan-4- yloxy)-phenyl ester; compound with form aldehyde(BAY-38-7271) | 8.0 | 7.3 | 6.0 | 5.2 |
| 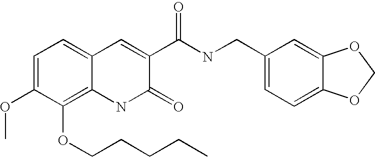 | 7-Methoxy-2-oxo-8- pentyloxy-1,2-di- hydro-quinoline-3-carboxylic acid (benzo[1,3]dioxol-5- ylmethyl)-amide (JTE-907) | 6.0 | 6.9 | 6.1 | 5.9 |
| 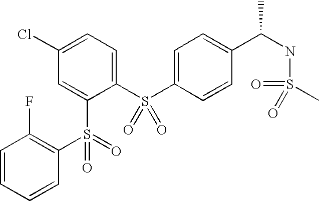 | N-(1-{4-[4-Chloro-2-(2- fluoro-benzene sulfonyl)- benzenesulfonyl]-phenyl}- ethyl)- methanesulfonamide (Schering) | 6.0 | 9.3 | 5.9 | 4.7 |
| 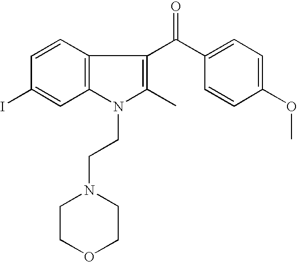 | [6-lodo-2-methyl-1-(2- morpholin-4-yl- ethyl)-2,3-dihydro-1H- indol-3-yl]- (4-methoxy-phenyl)- methanone (AM-630) | 6.7 | 7.6 | 5.9 | 5.0 |
| 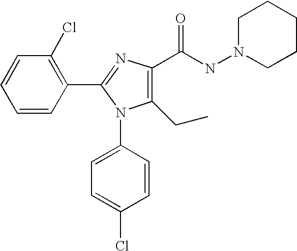 | 1-(4-Chloro-phenyl)-2-(2- chloro-phenyl)-5-ethyl-1H- imidazole-4-carboxylic acid piperidin-1-ylamide (Bayer) | 7.9 | 6.0 | n/a | 4.8 |
| 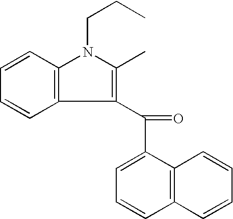 | (2-Methyl-1-propyl-2,3- dihydro-1H- indol-3-yl)-naphthalen-1-yl- (JWH-015) | 6.3 | 6.9 | 6.1 | 5.8 |
| 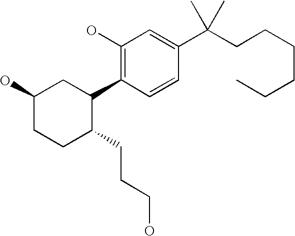 | 5-(1,1-Dimethyl-heptyl)-2- [5-hydroxy-2-(3-hydroxy- propyl)-cyclohexyl]- phenol (CP55940) | 9.0 | 9.3 | 5.3 | 5.2 |
| 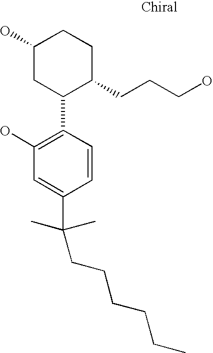 | 5-(1,1-Dimethyl-heptyl)-2- [5-hydroxy-2-(3-hydroxy- propyl)-cyclohexyl]- phenol (CP55940-entantiomer) | 7.2 | 7.0 | 5.3 | 5.3 |
| 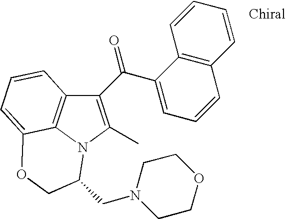 | (2-Methyl-3-morpholin-4- ylmethyl-3,4-dihydro-5- oxa-2a-azacenaphthylen- 1-yl)-naphthalen-1-yl- methanone (R(+)-WIN55212-2) | 7.1 | 8.1 | 4.4 | 5.4 |
| 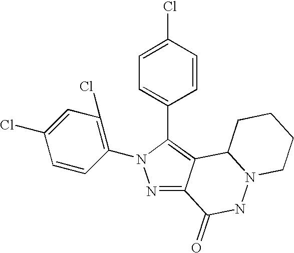 | 5-(4-Chloro-phenyl)-1-(2,4- dichloro-phenyl)-4-methyl- 1H-pyrazole-3-carboxylic acid piperidin-1-ylamide (Rimonabant) | 8.2 | 6.0 | 5.3 | 5.4 |
| 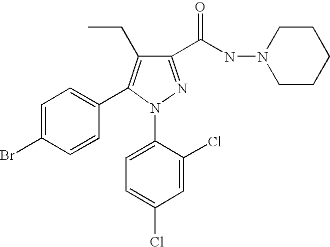 | 5-(4-Bromo-phenyl)-1-(2,4- dichloro-phenyl)-4-ethyl- 1H-pyrazole-3-carboxylic acid piperidin-1-ylamide (SR-147778) | 7.9 | 5.9 | 5.5 | 5.6 |
| 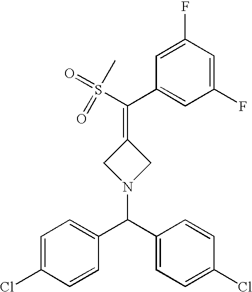 | 1-[Bis-(4-chloro-phenyl)- methyl]-3-[(3,5-difluoro- phenyl)-methanesulfonyl- methylene]-azetidine (Aventis) | 8.2 | n/a | 5.3 | 5.1 |
| 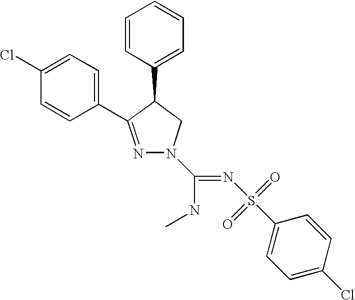 | 4-Chloro-N-{[3-(4-chloro- phenyl)-4-phenyl-4,5- dihydro-pyrazol-1-yl]- methylamino-methylenel- benzenesulfonamide | 8.4 | n/a | 6.3 | 5.4 |
| 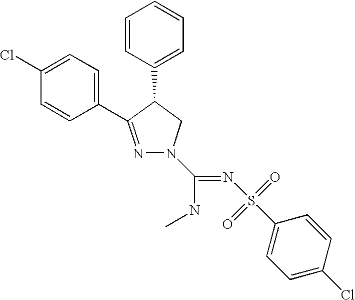 | 4-Chloro-N-{[3-(4-chloro- phenyl)-4-phenyl-4,5- dihydro-pyrazol-1-yl]- methylamino-methylene}- benzenesulfonamide | 6.3 | 5.7 | 6.0 | 6.1 |
| 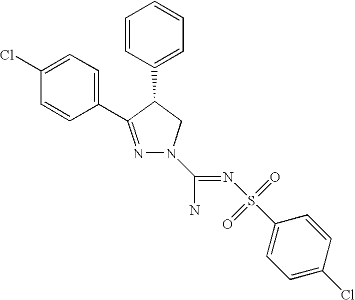 | N-{Amino-[3-(4-chloro- phenyl)-4-phenyl-4,5- dihydro-pyrazol-1-yl]- methylene}-4-chloro- benzenesulfonamide | 8.4 | 6.8 | 6.1 | 5.4 |
| 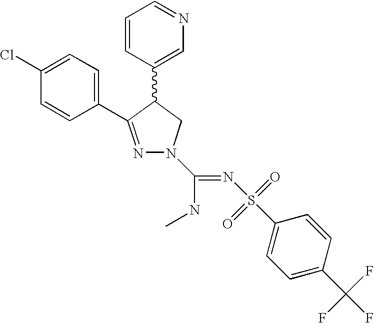 | N-([3-(4-Chloro-phenyl)-4- pyridin-3-yl-4,5-dihydro- pyrazol-1-yl]-methylamino- methylene}-4- trifluoromethyl- benzenesulfonamide | 8.2 | — | 6.3 | 4.9 |
| 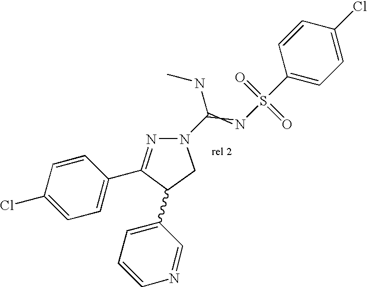 | 4-Chloro-N-{[3-(4-chloro- phenyl)-4-pyridin-3-yl-4,5- dihydro-pyrazol-1-yl]- methylamino-methylenel- benzenesulfonamide | 7.1 | n/a | 5.6 | 5.4 |
| 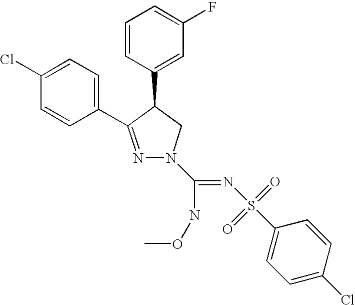 | 4-Chloro-N-{[3-(4-chloro- phenyl)-4-(3-fluoro- phenyl)-4,5-dihydro- pyrazol- 1-yl]- methoxyamino- methylene}- benzenesulfonamide | 7.7 | n/a | 5.6 | 6.1 |
| 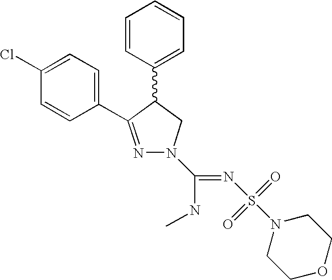 | Morpholine-4-sulfonic acid [3-(4-chloro-phenyl)-4- phenyl-4,5-dihydro- pyrazol-1-yl]-methylamino- methyleneamide | 8.3 | n/a | 6.3 | 4.0 |
| 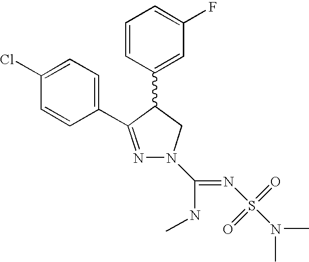 | N-{[3-(4-Chloro-phenyl)-4- (3-fluoro-phenyl)-4,5- dihydro-pyrazol-1-yl]- methylamino-methylene}- N,N-dimethyl-sulfonamide | 8.5 | n/a | 7.0 | 5.3 |
| 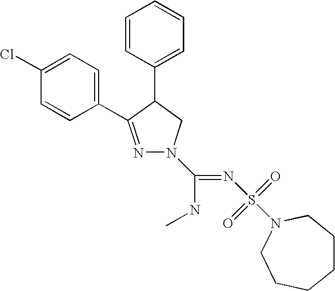 | Azepane-1-sulfonic acid [3-(4-chloro-phenyl)-4- phenyl-4,5-dihydro- pyrazol-1-yl]-methylamino- methyleneamide | 7.3 | n/a | 4.6 | 4.9 |
| 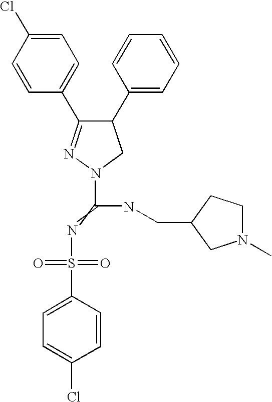 | 4-Chloro-N-{[3-(4-chloro- phenyl)-4-phenyl-4,5- dihydro-pyrazol-1-yl]- [(1-methyl-pyrrolidin-3- ylmethyl)-amino]- methylene]- benzenesulfonamide | 9.0 | 6.0 | 6.0 | 4.8 |
| 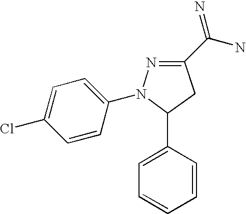 | 1-(4-Chloro-phenyl)-5- phenyl-4,5-dihydro-1H- pyrazole-3-carboxamidine | 6.2 | 6.3 | 4.0 | 4.0 |
| 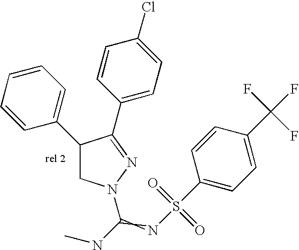 | N-{[3-(4-Chloro-phenyl)-4- phenyl-4,5-dihydro- pyrazol-1-yl]-methylamino- methylene}-4- trifluoromethyl-benzene- sulfonamide | 8.4 | n/a | 6.3 | 4.2 |
| 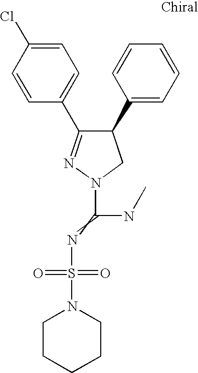 | Piperidine-1-sulfonic acid [3-(4-chloro-phenyl)-4- phenyl-4,5-dihydro- pyrazol-1-yl]-methylamino- methyleneamide | 8.1 | n/a | 5.8 | 4.3 |
| 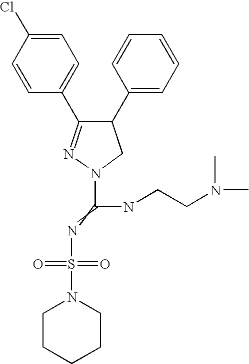 | Piperidine-1-sulfonic acid [3-(4-chloro-phenyl)-4- phenyl-4,5-dihydro- pyrazol-1-yl]-(2- dimethylamino-ethyl amino)-methyleneamide | 8.6 | n/a | 4.0 | 4.0 |
| 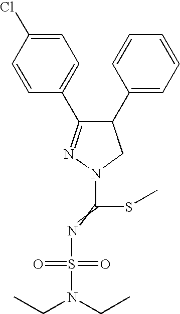 | N,N-Diethylamino-1- sulfonic acid [3-(4-chloro- phenyl)-4-phenyl-4,5- dihydro-pyrazol-1-yl]- methylsulfanyl- methyleneamide | 7.1 | n/a | 4.0 | 4.7 |
| 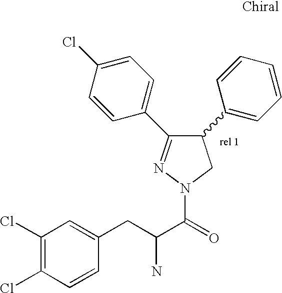 | 2-Amino-1-[3-(4-chloro- phenyl)-4-phenyl-4,5- dihydro-pyrazol-1-yl]-3- (3,4-dichloro-phenyl)- propan-1-one | 6.0 | 6.2 | 5.4 | 4.8 |
| 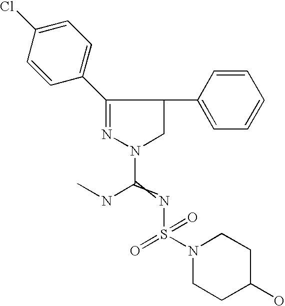 | Morpholine-4-sulfonic acid [3-(4-chloro-phenyl)-4- phenyl-4,5-dihydro- pyrazol-1-yl]-methylamino- methyleneamide | 7.7 | n/a | 4.0 | 5.5 |
| 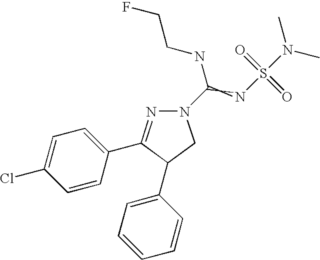 | N,N-Dimethylamino-1- sulfonic acid [3-(4-chloro- phenyl)-4-phenyl-4,5- dihydro-pyrazol-1-yl]-(2- fluoro-ethylamino)- methyleneamide | 7.5 | n/a | 4.3 | 4.0 |
| 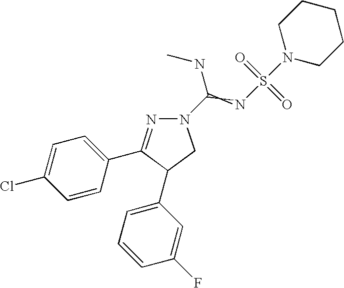 | Piperidine-1-sulfonic acid [3-(4-chloro-phenyl)-4-(3- fluoro-phenyl)-4,5-dihydro- pyrazol-1-yl]-methylamino- methyleneamide | 7.5 | 6.1 | 4.5 | 6.3 |
| 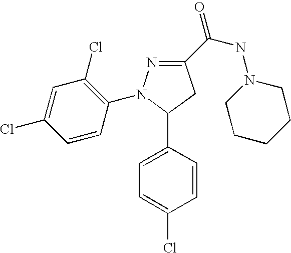 | 5-(4-Chloro-phenyl)-1-(2,4- dichloro-phenyl)-4,5- dihydro-1H-pyrazole-3- carboxylic acid piperidine- 1-ylamide | 7.4 | 6.1 | 5.4 | 5.6 |
| 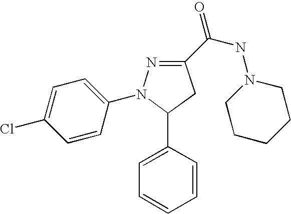 | 1-(4-Chloro-phenyl)-5- phenyl-4,5-dihydro-1H- pyrazole-3-carboxylic acid piperidin-1-ylamide | 7.2 | n/a | 4.0 | 5.1 |
| 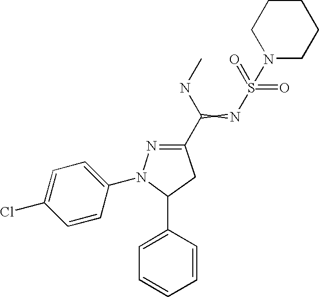 | Piperidine-1-sulfonic acid [1-(4-chloro-phenyl)-5- phenyl-4,5-dihydro-1H- pyrazol-3-yl]- methylamino- methyleneamide | 7.3 | n/a | 6.1 | 4.5 |
| 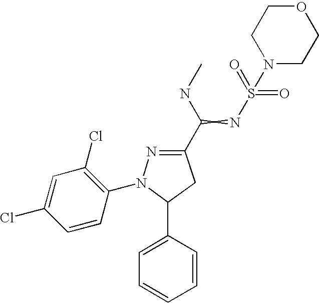 | Morpholine-4-sulfonic acid [1-(2,4-dichloro-phenyl)-5- phenyl-4,5-dihydro-1H- pyrazol-3-yl]-methylamino- methyleneamide | 6.9 | n/a | 5.3 | 5.0 |
| 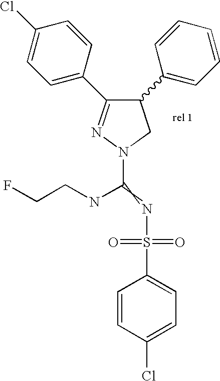 | 4-Chloro-N-[[3-(4-chloro- phenyl)-4-phenyl-4,5- dihydro-pyrazol-1-yl]-(2- fluoro-ethylamino)- methylene]- benzenesulfonamide | 7.7 | n/a | 4.6 | 4.6 |
| 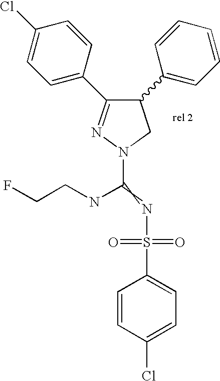 | 4-Chloro-N-113-(4-chloro- phenyl)-4-pheflyl-4,5- dihydro-pyrazol- 1-yl]-(2- fluoro-ethylamino)- methylene]- benzenesulfonamide | 6.9 | n/a | 4.6 | 5.0 |
| 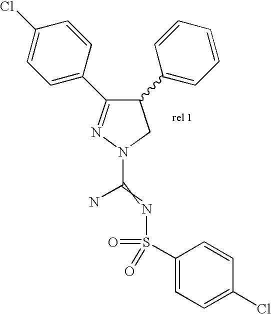 | N-{Amino-[3-(4-chloro- phenyl)-4-phenyl-4,5- dihydro-pyrazol-1-yl]- methylene}-4-chloro- benzenesulfonamide | 7.4 | 6.4 | 6.0 | 5.1 |
| 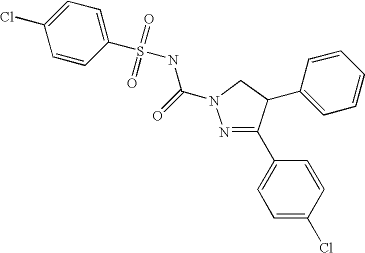 | 4-Chloro-N-[3-(4-chloro- phenyl)-4-phenyl-4,5- dihydro-pyrazole-1- carbonyl]- benzenesulfonamide | 6.5 | n/a | 5.5 | 4.8 |
| 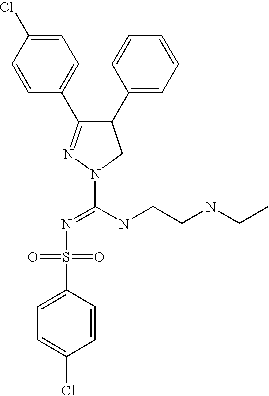 | 4-Chloro-N-[[3-(4-chloro- phenyl)-4-phenyl-4,5- dihydro-pyrazol-1-yl]-(2- ethylamino-ethylamino)- methylene]- Benzenesulfonamide | 8.1 | n/a | 5.1 | 5.3 |
| 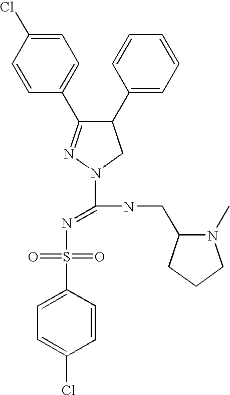 | 4-Chloro-N-{[3-(4-chloro- phenyl)-4-phenyl-4,5- dihydro-pyrazol-1-yl]-[(1- methyl-pyrrolidin-2- ylmethyl)-amino]- methylene}- benzenesulfonamide | 8.3 | n/a | n/a | 5.1 |
| 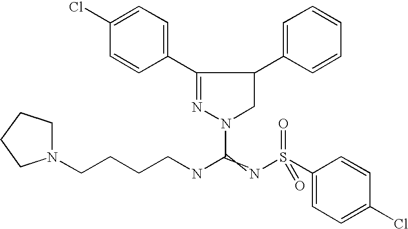 | 4-Chloro-N-[[3-(4-chloro- phenyl)-4-phenyl-4,5- dihydro-pyrazol-1-yl]-(4- pyrrolidin-1-yl- butylamino)-methylene]- benzenesulfonamide | 7.4 | n/a | 4.3 | 5.4 |
| 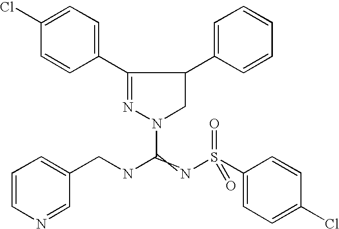 | 4-Chloro-N-{[3-(4-chloro- phenyl)-4-phenyl-4,5- dihydro-pyrazol-1-yl]-[(pyridin- 3-ylmethyl)-amino]- methylene}- benzenesulfonamide | 6.4 | 6.3 | 5.9 | 5.7 |
| 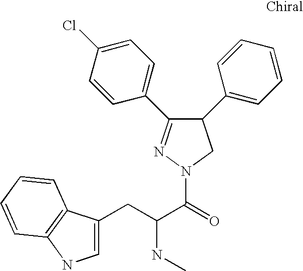 | 1-[3-(4-Chloro-phenyl)-4- phenyl-4,5-dihydro- pyrazol-1-yl]-3-(1H-indol- 2-yl)-2-methylamino- propan-1-one | 8.0 | 6.9 | 6.6 | 4.6 |
| 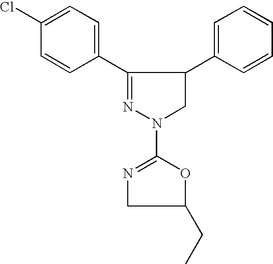 | 2-[3-(4-Chloro-phenyl)-4- phenyl-4,5-dihydro- pyrazol-1-yl]-5-ethyl-4,5- dihydro-oxazole | 6.2 | n/a | 5.5 | 4.4 |
| 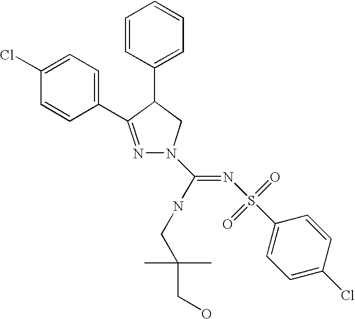 | 4-Chloro-N-[[3-(4-chloro- phenyl)-4-phenyl-4,5- dihydro-pyrazol-1-yl]-(3- hydroxy-2,2-dimethyl- propylamino)- methylene]- benzenesulfonamide | 6.7 | 6.1 | 4.5 | 5.4 |
| 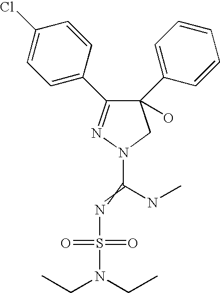 | N,N-Diethylamino-1- sulfonic acid [3-(4- chloro-phenyl)-4-hydroxy-4- phenyl-4,5-dihydro- pyrazol-1-yl]-methylamino- methyleneamide | 7.4 | n/a | 4.0 | 5.9 |
| 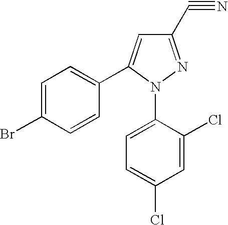 | 5-(4-Bromo-phenyl)-1-(2,4- dichloro-phenyl)-1H- pyrazole-3-carbonitrile | 6.3 | n/a | 4.0 | 5.4 |
| 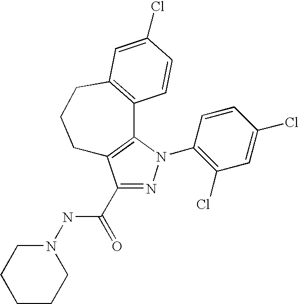 | 8-Chloro-1-(2,4-dichloro- phenyl)-1,3a,4,5,6,10b- hexahydro-1,2-diaza- benzo[e]azulene-3-carboxylic acid piperidin-1-ylamide | 6.9 | 6.9 | 5.5 | 4.0 |
| 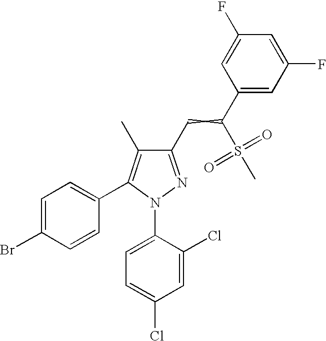 | 5-(4-Bromo-phenyl)-1-(2,4- dichloro-phenyl)-3-(2-(3,5- difluoro-phenyl)-2- methanesulfonyl-vinyl]-4- methyl-1H-pyrazole | 7.3 | 5.9 | 4.0 | 4.0 |
| 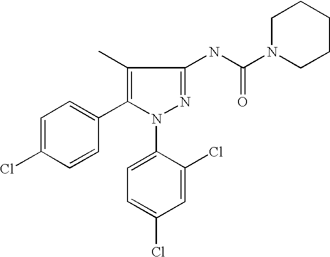 | Piperidine-1-carboxylic acid [5-(4-chloro-phenyl)-1- (2,4-dichloro-phenyl)-4- methyl-1H-pyrazol-3-yl]- amide | 6.9 | n/a | 4.7 | 4.7 |
| 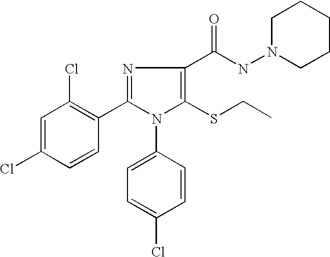 | 1-(4-Chloro-phenyl)-2-(2,4- dichloro-phenyl)-5- ethylsulfanyl-1H-imidazole- 4-carboxylic acid piperidin- 1-ylamide | 7.4 | n/a | 5.1 | 5.3 |
| 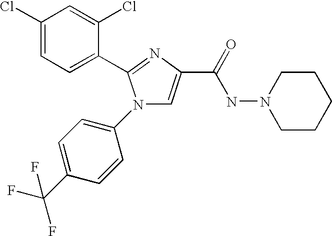 | 2-(2,4-Dichloro-phenyl)-1- (4-trifluoromethyl-phenyl)- 1H-imidazole-4-carboxylic acid piperidin-1-ylamide | 7.6 | n/a | 5.6 | 5.3 |
| 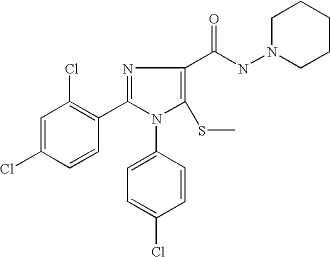 | 1-(4-Chloro-phenyl)-2-(2,4- dichloro-phenyl)-5- methylsulfanyl-1H-imidazole- 4-carboxylic acid piperidin-1-ylamide | 8.0 | 6.0 | 5.1 | 5.6 |
| 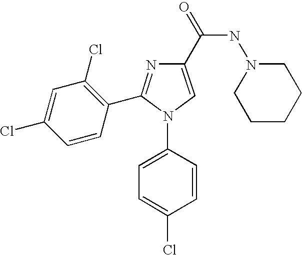 | 1-(4-Chloro-phenyl)-2-(2,4- dichloro-phenyl)-1H- imidazole-4-carboxylic acid piperidin-1-ylamide | 8.1 | 6.2 | 4.5 | 5.3 |
| 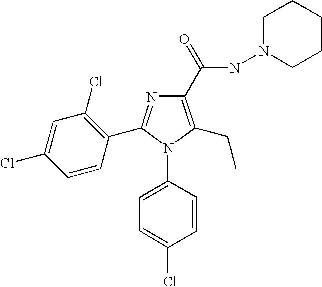 | 1-(4-Chloro-phenyl)-2-(2,4- dichloro-phenyl)-5-ethyl- 1H-imidazol-4-carboxylic acid piperidin-1-ylamide | 8.7 | 6.3 | 5.3 | 5.5 |
| 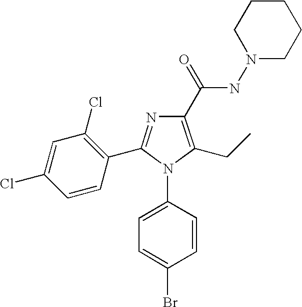 | 1-(4-Bromo-phenyl)-2-(2,4- dichloro-phenyl)-5-ethyl- 1H-imidazol-4-carboxylic acid piperidin-1-ylamide | 7.5 | n/a | 5.4 | 5.3 |
| 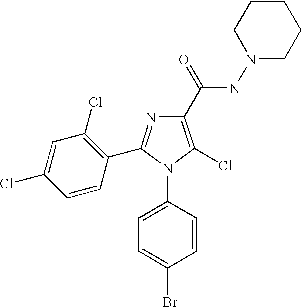 | 1-(4-Bromo-phenyl)-5- chloro-2-(2,4-dichloro- phenyl)-1H-imidazole-4- carboxylic acid piperidin-1- ylamide | 8.6 | n/a | 5.4 | 5.5 |
| 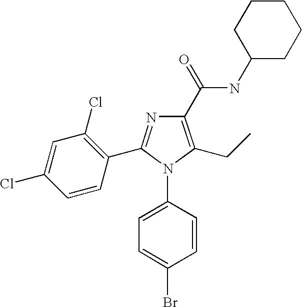 | 1-(4-Bromo-phenyl)-2-(2,4- dichloro-phenyl)-5-ethyl- 1H-imidazole-4-carboxylic acid cyclohexylamide | 7.0 | 6.7 | 4.0 | 5.1 |
| 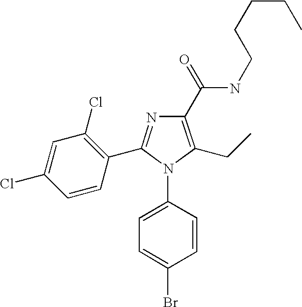 | 1-(4-Bromo-phenyl)-2-(2,4- dichloro-phenyl)-5-ethyl- 1H-imidazole-4-carboxylic acid pentylamide | 7.5 | n/a | 6.1 | 5.6 |
| 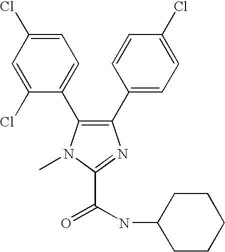 | 4-(4-Chloro-phenyl)-5-(2,4- dichloro-phenyl)-1-methyl- 1H-imidazole-2-carboxylic acid cyclohexylamide | 7.2 | n/a | 4.0 | 4.7 |
| 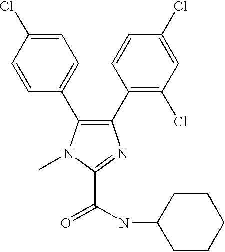 | 4-(4-Chloro-phenyl)-5-(2,4- dichloro-phenyl)-3-methyl- 1H-imidazole-2-carboxylic acid cyclohexylamide | 7.1 | n/a | 4.0 | 4.9 |
| 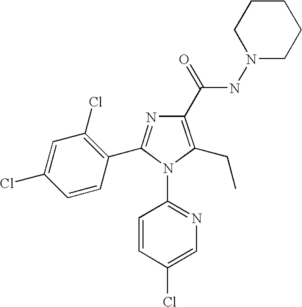 | 1-(5-Chloro-pyridin-2-yl)-2- (2,4-dichloro-phenyl)-5- ethyl-1H-imidazole piperidin-1-ylamide | 7.6 | n/a | 4.0 | 4.4 |
| 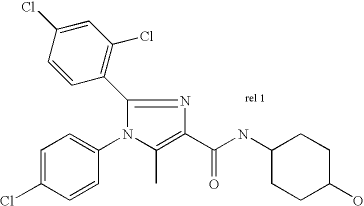 | 1-(4-Chloro-phenyl)-2-(2,4- dichloro-phenyl)-5-methyl- 1H-imidazole-4-carboxylic acid (4-hydroxy-cyclo- hexyl)-amide | 6.6 | n/a | 4.0 | 5.0 |
| 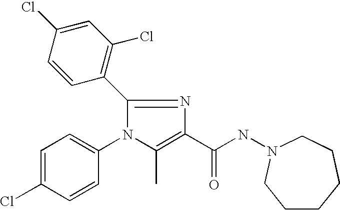 | 1-(4-Chloro-phenyl)-2-(2,4- dichloro-phenyl)-5-methyl- 1H-imidazole-4-carboxylic acid azepan-1-ylamide | 7.2 | 7.6 | 5.2 | 5.7 |
| 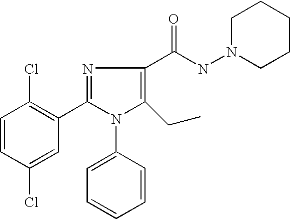 | 2-(2,4-Dichloro-phenyl)-5- ethyl-1-phenyl-1H- imidazole-4-carboxylic acid piperidin-1-ylamide | 7.0 | 7.6 | 4.0 | 5.3 |
| 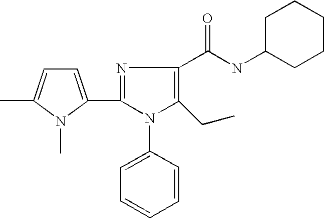 | 2-(1,5-Dimethyl-1H-pyrrol- 2-yl)-5-ethyl-1-phenyl-1H- imidazole-4-carboxylic acid cyclohexylamide | n/a | 6.8 | 4.0 | 4.0 |
| 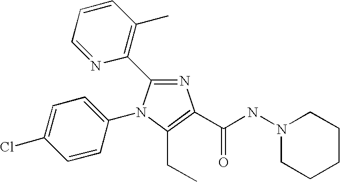 | 1-(4-Chloro-phenyl)-5- ethyl-2-(3-methyl-pyridin-2- yl)-1H-imidazole-4- carboxylic acid piperidin-1- ylamide | n/a | 6.6 | 4.0 | 4.8 |
| 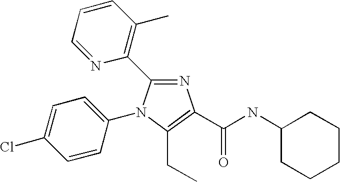 | 1-(4-Chloro-phenyl)-5- ethyl-2-(3-methyl-pyridin-2- yl)-1H-imidazole-4- carboxylic acid cyclohexylamide | n/a | 6.1 | 4.0 | 5.5 |
| 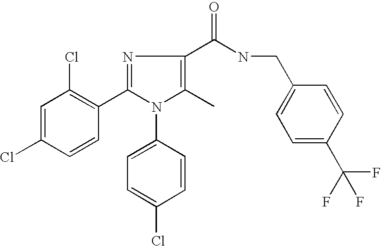 | 1-(4-Chloro-phenyl)-2-(2,4- dichloro-phenyl)-5-methyl- 1H-imidazole-4-carboxylic acid (4-trifluoromethyl- phenyl)-amide | 6.7 | n/a | 4.5 | 5.3 |
| 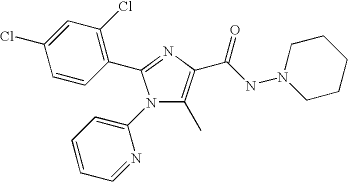 | 2-(2,4-Dichloro-phenyl)-5- methyl-1-pyridin-2-yl-1H- imidazole-4-carboxylic acid piperidin-1-ylamide | 7.3 | 6.2 | 4.0 | 4.1 |
| 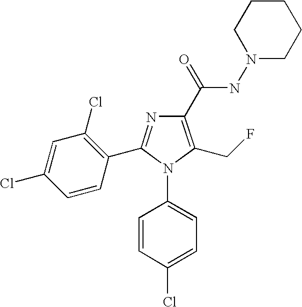 | 1-(4-Chloro-phenyl)-2-(2,4- dichloro-phenyl)-5- fluoromethyl-1H-imidazole- 4-carboxylic acid piperidin-1-ylamide | 7.2 | 6.1 | 5.2 | 5.5 |
| 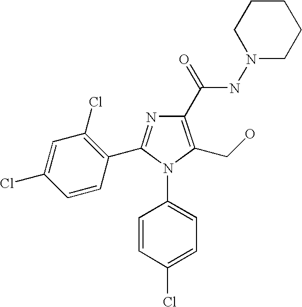 | 1-(4-Chloro-phenyl)-2-(2,4- dichloro-phenyl)-5- hydroxymethyl-1H-imidazole- 4-carboxylic acid piperidin-1-ylamide | 7.6 | 6.2 | 4.0 | 4.7 |
| 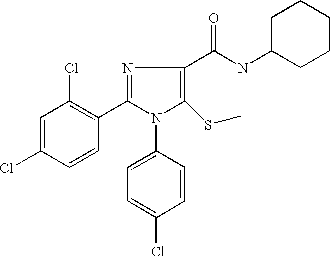 | 1-(4-Chloro-phenyl)-2-(2,4- dichloro-phenyl)-5- methylsulfanyl-1H-imidazole- 4-carboxylic acid cyclohexylamide | 7.2 | 6.6 | 6.3 | 5.8 |
| 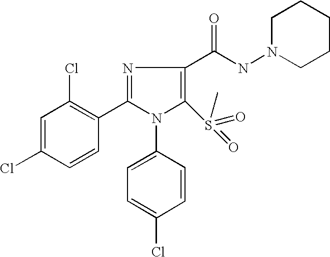 | 1-(4-Chloro-phenyl)-2-(2,4- dichloro-phenyl)-5- methanesulfonyl-1H-imidazole- 4-carboxylic acid piperidin-1-ylamide | 7.8 | n/a | 4.0 | 5.0 |
| 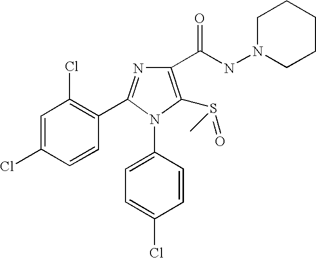 | 1-(4-Chloro-phenyl)-2-(2,4- dichloro-phenyl)-5- methanesulfinyl-1H-imidazole- 4-carboxylic acid piperidin-1-ylamide | 7.4 | n/a | 4.0 | 4.9 |
| 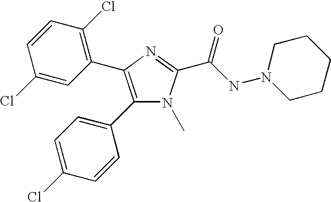 | 5-(4-Chloro-phenyl)-4-(2,5- dichloro-phenyl)-1-methyl 1H-imidazole-2-carboxylic acid piperidin-1-ylamide | 6.7 | 6.4 | 4.6 | 5.0 |
| 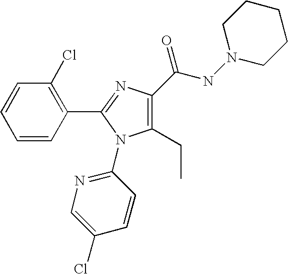 | 2-(2-Chloro-phenyl)-1-(5- chloro-pyridin-2-yl)-5-ethyl- 1H-imidazole-4-carboxylic acid piperidin-1-ylamide | 7.9 | n/a | 4.0 | 4.8 |
| 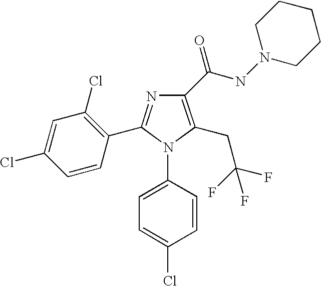 | 1-(4-Chloro-phenyl)-2-(2,4- dichloro-phenyl)-5-(2,2,2- trifluoro-ethyl)-1H- imidazole-4-carboxylic acid piperidin-1-ylamide | 7.5 | n/a | 4.8 | 5.7 |
| 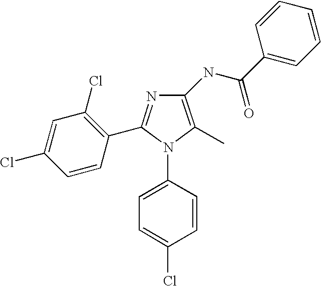 | N-[1-(4-Chloro-phenyl)-2- (2,4-dichloro-phenyl)-5- methyl-1H-imidazol-4-yl]- benzamide | 7.0 | 6.3 | 5.7 | 5.2 |
| 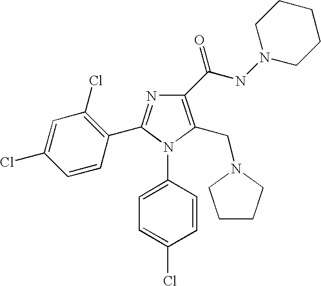 | 1-(4-Chloro-phenyl)-2-(2,4- dichloro-phenyl)-5- pyrrolidin-1-ylmethyl-1H- imidazole-4-carboxylic acid piperidin-1-ylamide | 6.3 | n/a | 4.0 | 4.6 |
| 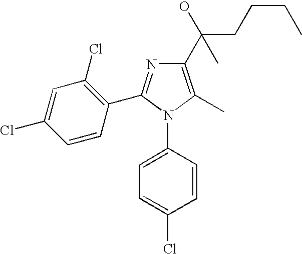 | 2-[1-(4-Chloro-phenyl)-2- (2,4-dichloro-phenyl)-5- methyl-1H-imidazol-4-yl]- hexan-2-ol | 6.6 | n/a | 5.8 | 5.3 |
| 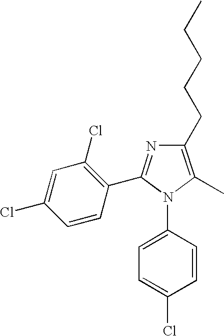 | 1-(4-Chloro-phenyl)-2-(2,4- dichloro-phenyl)-5-methyl- 4-pentyl-1H-imidazole | 7.0 | n/a | 5.8 | 5.3 |
| 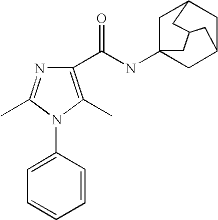 | 2,5-Dimethyl-1-phenyl-1H- imidazole- 4-carboxylic acid adamantan-2-ylamide | n/a | 9.0 | 6.0 | 4.6 |
| 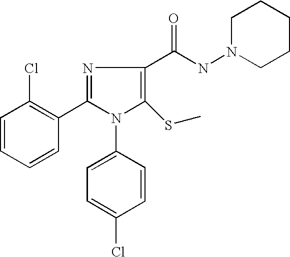 | 1-(4-Chloro-phenyl)-2-(2- chloro-phenyl)-5- methylsulfanyl-1H- imidazole-4-carboxylic acid piperidin-1-ylamide | n/a | 5.2 | 8.2 | n/a |
| 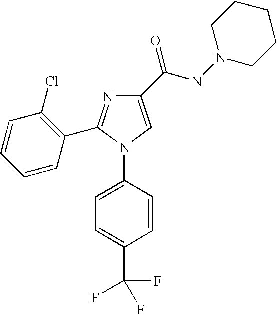 | 2-(2-Chloro-phenyl)-1-(4- trifluoromethyl-phenyl)-1H- imidazole-4-carboxylic acid piperidin-1-ylamide | 7.0 | n/a | 6.0 | 5.7 |
| 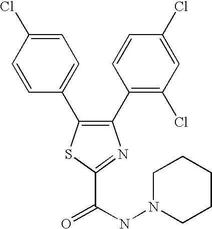 | 5-(4-Chloro-phenyl)-4-(2,4- dichloro-phenyl)-thiazole- 2-carboxylic acid piperidin- 1-ylamide | 6.7 | n/a | 5.7 | 5.5 |
| 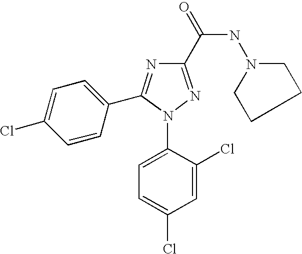 | 5-(4-Chloro-phenyl)-1-(2,4- dichloro-phenyl)-1H- [1,2,4]triazole-3-carboxylic acid pyrrolidin-1-ylamide | 6.6 | n/a | 4.0 | 4.4 |
| 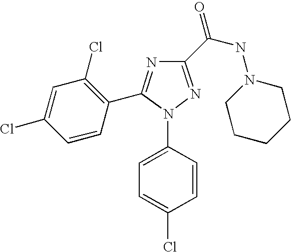 | 1-(4-Chloro-phenyl)-5-(2,4- dichloro-phenyl)-1H- [1,2,4]triazole-3-carboxylic acid piperidin-1-yl-amide | 6.5 | n/a | 5.0 | 5.0 |
| 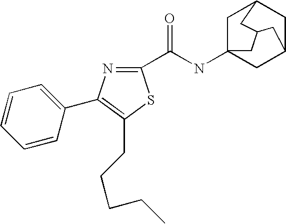 | 5-Pentyl-4-phenyl-thiazole- 2-carboxylic acid (hexahydro-2,5-methano- pentalen-3a-yl)-amide | 6.4 | 7.0 | 4.0 | 4.0 |
| 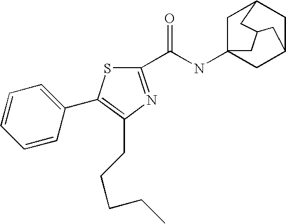 | 4-Pentyl-5-phenyl-thiazole- 2-carboxylic acid (hexahydro-2,5-methano- pentalen-3a-yl)-amide | 7.8 | 8.1 | 4.0 | 4.0 |
| 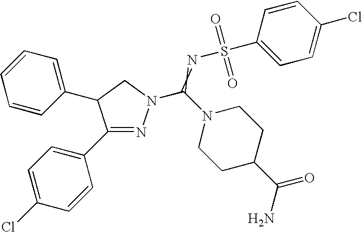 | 1-{(4-Chloro-benzene- sulfonylimino)-[3-(4-chloro- phenyl)-4-phenyl-4,5- dihydro-pyrazol-1-yl]- methyl}-piperidine-4- carboxylic acid amide | 8.2 | n/a | 5.5 | 5.2 |
| 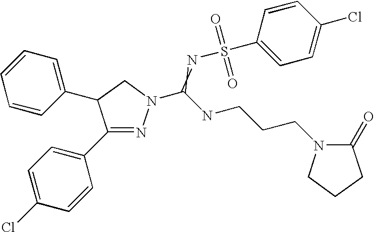 | 4-Chloro-N-{[3-(4-chloro- phenyl)-4-phenyl-4,5- dihydro-pyrazol- 1-yl]-[2-(2- oxo-pyrrolidin-1-yl)- ethylamino]-methylene}- benzenesulfonamide | 8.2 | n/a | 6.3 | 5.9 |
| 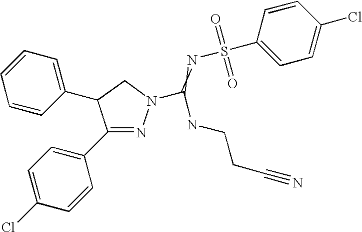 | 4-Chloro-N-[[3-(4-chloro- phenyl)-4-phenyl-4,5- dihydro-pyrazol-1-yl]-(2- cyano-ethylamino)- methylene]-benzene sulfonamide | 7.9 | n/a | 6.7 | 5.9 |
| 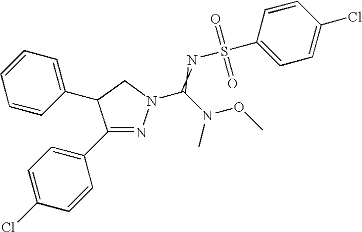 | 4-Chloro-N-[[3-(4-chloro- phenyl)-4-phenyl-4,5- dihydro-pyrazol-1-yl]- (methoxy-methyl-amino)- methylene]- benzenesulfonamide | 6.7 | n/a | 5.9 | 5.9 |
| 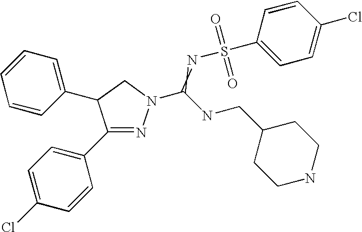 | 4-Chloro-N-{[3-(4-chloro- dihydro-pyrazol-1-yl]- [(piperidin-4-ylmethyl)- amino]-methylene}- benzenesulfonamide | 8.6 | n/a | 6.2 | 4.9 |
| 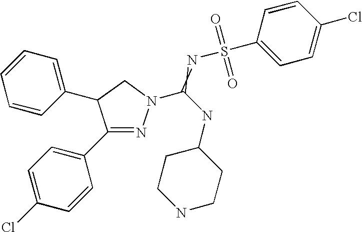 | 4-Chloro-N-[[3-(4-chloro- phenyl)-4-phenyl-4,5- dihydro-pyrazol-1-yl]- (piperidin-4-ylamino)- methylene]- benzenesulfonamide | 8.2 | n/a | 4.5 | 4.6 |
| 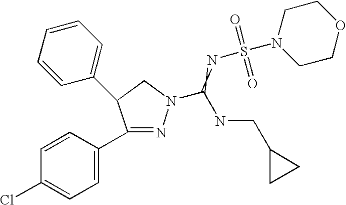 | Morpholine-4-sulfonic acid [3-(4-chloro-phenyl)-4- phenyl-4,5-dihydro- pyrazol-1-yl]- (cyclopropylmethyl-amino) methyleneamide | 8.5 | n/a | 6.0 | 5.7 |

The data in Table 1 demonstrate that the tested CBₓ modulators act selectively on the SUR1 subunit and/or on the SUR 2 subunit.

Compounds included within the scope of the present disclosure have an in vitro binding affinity for the cannabinoid CB₁ receptor (measured as pKᵢ) between about 6.0 and about 9.0, between about 5.0 and about 10.0, between about 4.0 and about 12.0, between about 7.0 and about 9.0 and between about 8.0 and about 9.0 using membrane preparations of CHO cells on which the human cannabinoid CB₁ receptor is stably transfected in conjunction with [³H]CP-55,940 as a radioligand as described herein. Further compounds include those with a binding affinity for the cannabinoid CB₁ receptor (measured as pKᵢ) using the previously described method of greater than about 2.0, greater than about 3.0, greater than about 4.0, greater than about 5.0, greater than about 6.0, greater than about 7.0, greater than about 8.0, greater than about 9.0, greater than about 10.0, greater than about 11.0 and greater than about 12.0. Further compounds included within the scope of the present disclosure are those with a binding affinity for the cannabinoid CB₁ receptor (measured as pKᵢ) using the previously described method of about 4.0, about 5.0, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0 about 10, about 11.0 and about 12.0.

Compounds included within the scope of the present disclosure have an in vitro binding affinity for the cannabinoid CB₂ receptor (measured as pKᵢ) between about 5.0 and about 10.0, between about 4.0 and about 12.0, between about 3.0 and about 12.0, between about 7.0 and about 10.0 and between about 8.0 and about 10.0 using membrane preparations of CHO cells on which the human cannabinoid CB₂ receptor is stably transfected in conjunction with [³H]CP-55,940 as a radioligand as described herein. Further compounds include those with a binding affinity for the cannabinoid CB₂ receptor (measured as pKᵢ) using the previously described method of greater than about 2.0, greater than about 3.0, greater than about 4.0, greater than about 5.0, greater than about 6.0, greater than about 7.0, greater than about 8.0, greater than about 9.0, greater than about 10.0, greater than about 11.0 and greater than about 12.0. Further compounds included within the scope of the present disclosure are those with a binding affinity for the cannabinoid CB₂ receptor (measured as pKᵢ) using the previously described method of about 4.0, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9 about 10, about 11.0 and about 12.0.

Compounds included within the scope of the present disclosure have an in vitro affinity for the binding sites for sulonylureas and K_{ATP} channel openers on hamster SUR1 (measured as pKᵢ) between about 3.0 and about 8.0, between about 2.0 and about 10.0, between about 1.0 and about 11.0, between about 4.0 and about 8.0 and between about 6.0 and about 9.0 using membrane from COS-cells transiently expressing hamster SUR1 incubated in the presence of [³H]glibenclamide with increasing amounts of the compound to be tested as described herein. Further compounds include those with an affinity for the binding sites for sulonylureas and K_{ATP} channel openers on hamster SUR1 (measured as pKᵢ) using the previously described method of greater than about 1.0, greater than about 2.0, greater than about 3.0, greater than about 4.0, greater than about 5.0, greater than about 6.0, greater than about 7.0, greater than about 8.0, greater than about 9.0, greater than about 10.0 and greater than about 11.0. Further compounds include those with an affinity for the binding sites for sulonylureas and K_{ATP} channel openers on hamster SUR1 (measured as pKᵢ) using the previously described method of about 1.0, about 2.0, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9 and about 9.0.

Compounds included within the scope of the present disclosure have an in vitro affinity for the binding sites for sulonylureas and K_{ATP} channel openers on rat SUR2 (measured as pKᵢ) between about 2.5 and about 7.0, between about 2.0 and about 8.0, between about 1.0 and about 9.0, between about 3.0 and about 7.0 and between about 5.0 and about 8.0 using membrane from COS-cells transiently expressing rat SUR2 incubated in the presence of [³H]glibenclamide with increasing amounts of the compound to be tested as described herein. Further compounds include those with an affinity for the binding sites for sulonylureas and K_{ATP} channel openers on rat SUR2 (measured as pKᵢ) using the previously described method of greater than about 1.0, greater than about 2.0, greater than about 3.0, greater than about 4.0, greater than about 5.0, greater than about 6.0, greater than about 7.0, greater than about 8.0, greater than about 9.0 and greater than about 10.0. Further compounds include those with an affinity for the binding sites for sulonylureas and K_{ATP} channel openers on rat SUR2 (measured as pKᵢ) using the previously described method of about 1.0, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 and about 8.0.

4. Determination of the K_{ATP} Opening Effects of Compounds Through Insulin Secretion in Rat Perifused Pancreatic Islets

Animals: Male Wistar rats in the weight range between 175 and 200 g were group housed in standard animal cages at a temperature of 21±2° C. and humidity of 55±10%. The animals were maintained on a 12 h light-dark cycle (lights on 06.00-18.00 h) with free access to standard rodent diet (B&K Universal Ltd standard rat and mouse diet (BK 001P), Beekay Feeds, B&K Universal Ltd, Hull, East Riding of Yorkshire) and tap water. The rats were conditioned to this environment for at least one week before experimentation.

Experimental procedures: After the rats were sacrificed, the branch of the bile duct leading to the liver and the duodenal end of the duct in the pancreas were clamped and the pancreas distended by injection of ice-cold 0.9 mg/ml collagenase solution into the bile duct. The pancreas were then removed and incubated statically for 10-12 min at 37° C. Following incubation, 10 ml of cold buffer was added and the suspension shaken vigorously by hand for 1 min. The islets were allowed to settle for 5 min on ice and washed three times using ice-cold buffer. Well-formed and good-sized islets from three rats were hand-picked (under a low power microscope) and pooled and a final selection of islet transferred to the perifusion apparatus. Oxygenated (95% O₂/5% CO₂) Gey & Gey buffer containing 1 mg/ml bovine serum albumin and 4 mM glucose were used throughout the experiment unless otherwise stated (see Dickinson et al. Eur. J. Pharmacol. 1997; 339: 69-76 for further details).

Compounds were either tested at the advised concentration or the solubility was determined in the experimental conditions and a maximum soluble drug concentration used for experiments (DMSO or ethanol will be used as the solvents at a maximum 0.1% in the assay buffer).

Two experiments were performed in parallel in two identical, independent sets of perifusion apparatus each consisting of sufficient number of chambers. Each chamber was loaded with 20 hand-picked islets. Islets were perifused for an initial 30 min period in media containing 4 mM glucose. Perifusate was then collected at 2 min intervals for the remainder of the experiment. After the first 10 min of the experiment (to collect baseline insulin values), the media in each chamber was switched to one containing 11 mM glucose and the relevant drug concentration/vehicle/diazoxide concentration and perifusate collected for a further 62 min to produce a total of 36 fractions for each chamber. Perifusate samples were then pooled to create three samples per chamber as follows: Baseline (4 mM): Samples 1-5 (first 10 minutes); 0-30 minutes (11 mM glucose): Samples 6-21; 30-60 minutes (11 mM glucose): Samples 22-36. Perifusate fractions were stored at −75° C. until required for insulin assay. Insulin content of fractions were assayed using a 96-well ELISA assay (Mercodia). Initial insulin assays were performed in triplicate on three pooled fractions from each chamber.

Drugs: All chemicals were obtained from Sigma (or other appropriate commercial supplier).

Result: The three islet preparations showed a consistent degree of glucose dependent insulin secretion. The mean insulin secretion at 11 mM glucose was 98.3±12.6 pg/islet/min and 130.4±22.0 pg/islet/min at 0-30 and 30-60 minutes, respectively. In the presence of 4 mM glucose this was significantly lower and was 3.8±0.6 pg/islet/min and 3.4±0.1 pg/islet/min at 0-30 and 30-60 minutes, respectively. Thus, insulin secretion was increased by 26 times and 38 times by 11 mM glucose at 0-30 and 30-60 minutes, respectively. Data were initially expressed as a simple mean of the three experiments for insulin secretion (pg/islet/min) and multiple t-tests (against the corresponding vehicle time period) used to determine potential significant effects of treatments. Alternatively, data were also calculated as a % vehicle effect for each experimental day. This latter approach was deemed to be the more powerful analysis as it corrected for the day to day variation in insulin release from the islets. Diazoxide significantly inhibited insulin secretion by an average of 55.3% (0-30 min) and 58.9% (30-60 min).

TABLE 2
KATP channel openers analyzed according to the procedure described
hereinabove and expressed as percent (%) inhibition.

| | | % Inhibition |
|---|---|---|
| Compound/name | Chemical name | 0-30/30-60 |
| 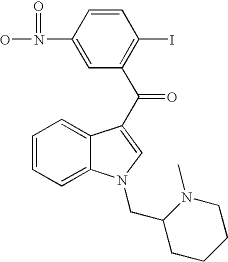 | (2-Iodo-5-nitro-phenyl)-[1- (1-methyl-piperidin-2- ylmethyl)-1H-indol-3-yl]- methanone (AM-1241) | 97/74 |
| 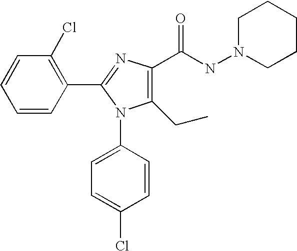 | 1-(4-Chloro-phenyl)-2-(2- chloro-phenyl)-5-ethyl-1H- imidazole-4-carboxylic acid piperidin-1-ylamide (Bayer) | 59/45 |
| 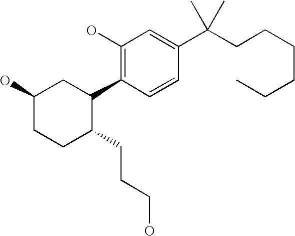 | 5-(1,1-Dimethyl-heptyl)-2- [5-hydroxy-2-(3-hydroxy- propyl)-cyclohexyl]- phenol (CP55940) | 91/56 |
| 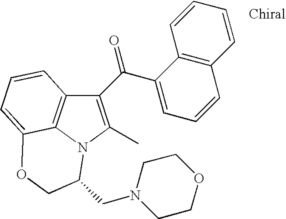 | (2-Methyl-3-morpholin-4- ylmethyl-3,4-dihydro-5- oxa-2a-azacenaphthylen- 1-yl)-naphthalen-1-yl- methanone (R(+)-WIN55212-2) | 80/35 |
| 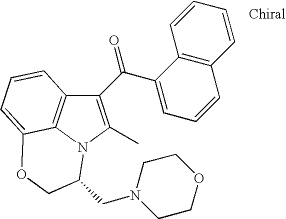 | (2-Methyl-3-morpholin-4- ylmethyl-3,4-dihydro-5- oxa-2a-azacenaphthylen- 1-yl)-naphthalen-1-yl- methanone (R(+)-WIN55212-2) | 69/40 |
| 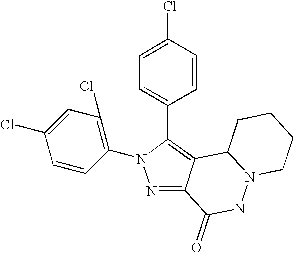 | 5-(4-Chloro-phenyl)-1-(2,4- dichloro-phenyl)-4-methyl- 1H-pyrazole-3-carboxylic acid piperidin-1-ylamide (Rimonabant) | 22/18 |
| 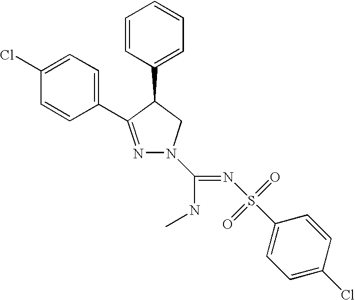 | 4-Chloro-N-{[3-(4-chloro- phenyl)-4-phenyl-4,5- dihydro-pyrazol-1-yl]- methylamino-methylenel- benzenesulfonamide | 42/22 |
| 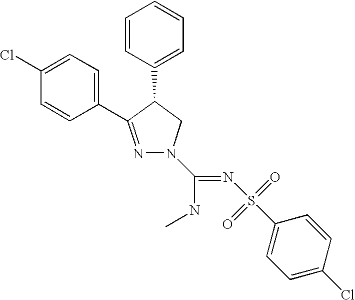 | 4-Chloro-N-{[3-(4-chloro- phenyl)-4-phenyl-4,5- dihydro-pyrazol-1-yl]- methylamino-methylene}- benzenesulfonamide | 59/25 |
| 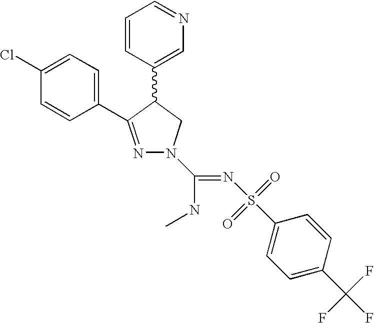 | N-([3-(4-Chloro-phenyl)-4- pyridin-3-yl-4,5-dihydro- pyrazol-1-yl]-methylamino- methylene}-4- trifluoromethyl- benzenesulfonamide | 58/31 |
| 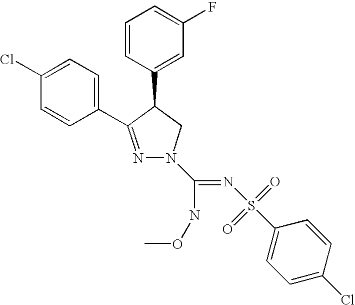 | 4-Chloro-N-{[3-(4-chloro- phenyl)-4-(3-fluoro- phenyl)-4,5-dihydro- pyrazol- 1-yl]- methoxyamino- methylene}- benzenesulfonamide | 90/95 |
| 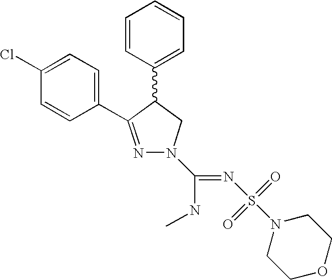 | Morpholine-4-sulfonic acid [3-(4-chloro-phenyl)-4- phenyl-4,5-dihydro- pyrazol-1-yl]-methylamino- methyleneamide | 83/91 |
| 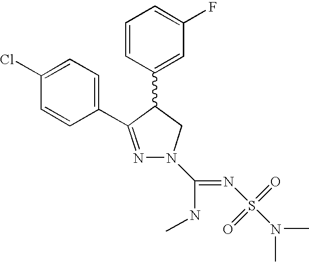 | N-{[3-(4-Chloro-phenyl)-4- (3-fluoro-phenyl)-4,5- dihydro-pyrazol-1-yl]- methylamino-methylene}- N,N-dimethyl-sulfonamide | 72/77 |
| 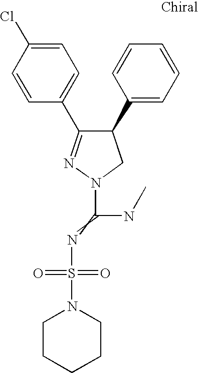 | Piperidine-1-sulfonic acid [3-(4-chloro-phenyl)-4- phenyl-4,5-dihydro- pyrazol-1-yl]-methylamino- methyleneamide | 49/40 |
| 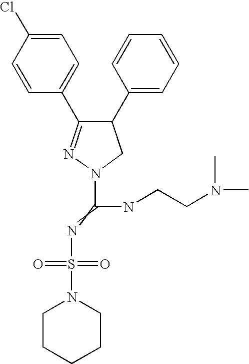 | Piperidine-1-sulfonic acid [3-(4-chloro-phenyl)-4- phenyl-4,5-dihydro- pyrazol-1-yl]-(2- dimethylamino-ethyl amino)-methyleneamide | 71/64 |
| 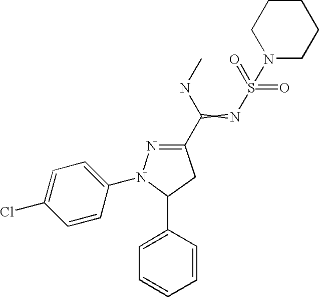 | Piperidine-1-sulfonic acid [1-(4-chloro-phenyl)-5- phenyl-4,5-dihydro-1H- pyrazol-3-yl]- methylamino- methyleneamide | 84/47 |
| 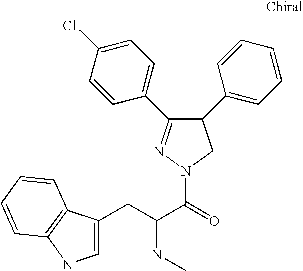 | 1-[3-(4-Chloro-phenyl)-4- phenyl-4,5-dihydro- pyrazol-1-yl]-3-(1H-indol- 2-yl)-2-methylamino- propan-1-one | 64/53 |
| 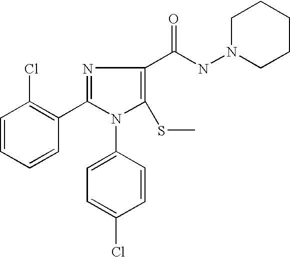 | 1-(4-Chloro-phenyl)-2-(2-chloro- phenyl)-5-methylsulfanyl-1H- imidazole-4-carboxylic acid piperidin-1-ylamide | 56/36 |

The tests described herein demonstrate that candidate compounds selected on the basis of their affinity for the K_{ATP} channel do inhibit glucose-stimulated insulin secretion. Moreover, the same candidate compounds also function as K_{ATP} channel openers under the conditions described hereinabove.

Suitable K_{ATP} channel opening compounds included within the scope of the present disclosure have a glucose-stimulated insulin secretion percent inhibition at 0-30 minutes in the experimental procedure set forth above of greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 855%, greater than about 90%, greater than about 91%, greater than about 92%, greater than about 93%, greater than about 94%, greater than about 95%, greater than about 96%, greater than about 97%, greater than about 98%, greater than about 99% and greater than about 99.5%.

Suitable K_{ATP} channel opening compounds included within the scope of the present disclosure have a glucose-stimulated insulin secretion percent inhibition at 30-60 minutes in the experimental procedure set forth above of greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 855%, greater than about 90%, greater than about 91%, greater than about 92%, greater than about 93%, greater than about 94%, greater than about 95%, greater than about 96%, greater than about 97%, greater than about 98%, greater than about 99% and greater than about 99.5%.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of any aspect of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or limitation at issue. The amount of broadening from the strict numerical boundary depends upon, many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

We claim:

1. A method of using a CBₓ modulator as a K_{ATP} channel modulator for the treatment of a medical condition in a mammal comprising the step of administering a pharmaceutically effective amount of a CBₓ modulator; wherein the CBₓ modulator is selected from the group consisting of: CB₁ agonists, CB₂ agonists, CB₂ partial agonists, CB₂ antagonists, CB₂ inverse agonists, compounds having both CB₁ agonist and CB₂ agonist properties, and mixtures thereof; and wherein the medical condition is selected from the group consisting of obesity, diabetes mellitus, metabolic syndrome, syndrome X, insulinoma, familial hyperinsulemic hypoglycemia, male pattern baldness, detrusor hyperreactivity, asthma, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain, idiopathic pain and impotence.

2. The method of claim 1 wherein the metabolic syndrome or syndrome X comprise disorders or diseases selected from the group consisting of: hypertension, insulin resistance, glucose intolerance and dyslipoproteinaemia.

3. The method of claim 1 wherein the CBₓ modulator is a modulator of at least one channel selected from the group consisting of: Kir6.2/SUR1K_{ATP}, Kir6.2/SUR2B K_{ATP}, Kir6.1/SUR2B K_{ATP}, and Kir6.2/SUR2A K_{ATP}.

4. The method of claim 1 wherein the K_{ATP} channel modulator is a K_{ATP} channel opener.

5. The method of claim 1 wherein the CBₓ modulator is selected from the group consisting of: 3-(1,1-dimethyl-butyl)-6,6,9-trimethyl-6a,7,10,10a-tetrahydro-6H-benzo[c]chromene; N-Adamantyl-4-pentyl-5-phenyl-thiazole-2-carboxamide; N-{1,3,3-Trimethyl-endo-(1S)-bicyclo[2.2.1]hept-2-yl}-1-[1-(4-methyl)-benzyl-5-(4-chloro-3-methyl-phenyl)-1H-pyrazol-3-carboxamide; (2-Iodo-5-nitro-phenyl)-[1-(1-methyl-piperidin-2-ylmethyl)-1H-indol-3-yl]-methanone; {4-[4-(1,1-Dimethyl-heptyl)-2,6-dimethoxy-phenyl]-6,6-dimethyl-bicyclo[3.1.1]hept-2-en-2-yl}-methanol; 3-(1,1-Dimethyl-heptyl)-9-hydroxymethyl-6,6-dimethyl-6a,7,10,10a-tetrahydro-6H-enzo[c]chromen-1-ol; Icosa-5,8, 11,14-tetraenoic acid 2-hydroxy-1-hydroxymethyl-ethyl ester; 1-Aziridin-1-yl-henicosa-6,9,12,15-tetraen-2-one; Noladineether; 4,4,4-Trifluoro-butane-1-sulfinic acid 3-(2-hydroxymethyl-indan-4-yloxy)-phenyl ester, compound with form aldehyde; 7-Methoxy-2-oxo-8-pentyloxy-1,2-dihydro-quinoline-3-carboxylic acid (benzo[1,3]dioxol-5-ylmethyl)-amide; N-(1-{4-[4-Chloro-2-(2-fluoro-benzenesulfonyl)-benzenesulfonyl]-phenyl}-ethyl)-methanesulfonamide; [6-Iodo-2-methyl-1-(2-morpholin-4-yl-ethyl)-2,3-dihydro-1H-indol-3-yl]-(4-methoxy-phenyl)-methanone; 1-(4-Chloro-phenyl)-2-(2-chloro-phenyl)-5-ethyl-1H-imidazole-4-carboxylic acid piperidin-1-ylamide; (2-Methyl-1-propyl-2,3-dihydro-1H-indol-3-yl)-naphthalen-1-yl-methanone; 5-(1,1-Dimethyl-heptyl)-2-[5-hydroxy-2-(3-hydroxy-propyl)-cyclohexyl]-phenol; (2-Methyl-3-morpholin-4-ylmethyl-3,4-dihydro-5-oxa-2a-azacenaphthylen-1-yl)-naphthalen-1-yl-methanone; 5-(4-Chloro-phenyl)-1-(2,4-dichloro-phenyl)-4-methyl-1H-pyrazole-3-carboxylic acid piperidin-1-ylamide; 5-(4-Bromo-phenyl)-1-(2,4-dichloro-phenyl)-4-ethyl-1H-pyrazole-3-carboxylic acid piperidin-1-ylamide; 1-[Bis-(4-chloro-phenyl)-methyl]-3-[(3,5-difluoro-phenyl)-methanesulfonyl-methylene]-azetidine; 4-Chloro-N-{[3-(4-chloro-phenyl)-4-phenyl-4,5-dihydro-pyrazol-1-yl]-methylamino-methylene}-benzenesulfonamide; N-{Amino-[3-(4-chloro-phenyl)-4-phenyl-4,5-dihydro-pyrazol-1-yl]-methylene}-4-chloro-benzenesulfonamide; N-{[3-(4-Chloro-phenyl)-4-pyridin-3-yl-4,5-dihydro-pyrazol-1-yl]-methylamino-methylene}-4-trifluoromethyl-benzenesulfonamide; 4-Chloro-N-{[3-(4-chloro-phenyl)-4-pyridin-3-yl-4,5-dihydro-pyrazol-1-yl]-methylamino-methylene}-benzenesulfonamide; 4-Chloro-N-{[3-(4-chloro-phenyl)-4-(3-fluoro-phenyl)-4,5-dihydro-pyrazol-1-yl]-methoxyamino-methylene}-benzenesulfonamide; Morpholine-4-sulfonic acid [3-(4-chloro-phenyl)-4-phenyl-4,5-dihydro-pyrazol-1-yl]-methylamino-methyleneamide; N-{[3-(4-Chloro-phenyl)-4-(3-fluoro-phenyl)-4,5-dihydro-pyrazol-1-yl]-methylamino-methylene}-N,N-dimethyl-sulfonamide; Azepane-1-sulfonic acid [3-(4-chloro-phenyl)-4-phenyl-4,5-dihydro-pyrazol-1-yl]-methylamino-methyleneamide; 4-Chloro-N-{[3-(4-chloro-phenyl)-4-phenyl-4,5-dihydro-pyrazol-1-yl]-[(1-methyl-pyrrolidin-3-ylmethyl)-amino]-methylene}-benzenesulfonamide; 1-(4-Chloro-phenyl)-5-phenyl-4,5-dihydro-1H-pyrazole-3-carboxamidine; N-{[3-(4-Chloro-phenyl)-4-phenyl-4,5-dihydro-pyrazol-1-yl]-methylamino-methylene}-4-trifluoromethyl-benzene-sulfonamide; Piperidine-1-sulfonic acid [3-(4-chloro-phenyl)-4-phenyl-4,5-dihydro-pyrazol-1-yl]-methylamino-methyleneamide; Piperidine-1-sulfonic acid [3-(4-chloro-phenyl)-4-phenyl-4,5-dihydro-pyrazol-1-yl]-(2-dimethylamino-ethylamino)-methyleneamide; N,N-Diethylamino-1-sulfonic acid [3-(4-chloro-phenyl)-4-phenyl-4,5-dihydro-pyrazol-1-yl]-methylsulfanyl-methyleneamide; 2-Amino-1-[3-(4-chloro-phenyl)-4-phenyl-4,5-dihydro-pyrazol-1-yl]-3-(3,4-dichloro-phenyl)-propan-1-one; Morpholine-4-sulfonic acid [3-(4-chloro-phenyl)-4-phenyl-4,5-dihydro-pyrazol-1-yl]-methylamino-methyleneamide; N,N-Dimethylamino-1-sulfonic acid [3-(4-chloro-phenyl)-4-phenyl-4,5-dihydro-pyrazol-1-yl]-(2-fluoro-ethylamino)-methyleneamide; Piperidine-1-sulfonic acid [3-(4-chloro-phenyl)-4-(3-fluoro-phenyl)-4,5-dihydro-pyrazol-1-yl]-methylamino-methyleneamide; 5-(4-Chloro-phenyl)-1-(2,4-dichloro-phenyl)-4,5-dihydro-1H-pyrazole-3-carboxylic acid piperidine-1-ylamide; 1-(4-Chloro-phenyl)-5-phenyl-4,5-dihydro-1H-pyrazole-3-carboxylic acid piperidin-1-ylamide; Piperidine-1-sulfonic acid [1-(4-chloro-phenyl)-5-phenyl-4,5-dihydro-1H-pyrazol-3-yl]-methylamino-methyleneamide; Morpholine-4-sulfonic acid [1-(2,4-dichloro-phenyl)-5-phenyl-4,5-dihydro-1H-pyrazol-3-yl]-methylamino-methyleneamide; 4-Chloro-N-[[3-(4-chloro-phenyl)-4-phenyl-4,5-dihydro-pyrazol-1-yl]-(2-fluoro-ethylamino)-methylene]-benzenesulfonamide; 4-Chloro-N-[[3-(4-chloro-phenyl)-4-phenyl-4,5-dihydro-pyrazol-1-yl]-(2-fluoro-ethylamino)-methylene]-benzenesulfonamide; N-{Amino-[3-(4-chloro-phenyl)-4-phenyl-4,5-dihydro-pyrazol-1-yl]-methylene}-4-chloro-benzenesulfonamide; 4-Chloro-N-[3-(4-chloro-phenyl)-4-phenyl-4,5-dihydro-pyrazole-1-carbonyl]-benzenesulfonamide; 4-Chloro-N-[[3-(4-chloro-phenyl)-4-phenyl-4,5-dihydro-pyrazol-1-yl]-(2-ethylamino-ethylamino)-methylene]-benzenesulfonamide; 4-Chloro-N-{[3-(4-chloro-phenyl)-4-phenyl-4,5-dihydro-pyrazol-1-yl]-[(1-methyl-pyrrolidin-2-ylmethyl)-amino]-methylene}-benzenesulfonamide; 4-Chloro-N-[[3-(4-chloro-phenyl)-4-phenyl-4,5-dihydro-pyrazol-1-yl]-(4-pyrrolidin-1-yl-butylamino)-methylene]-benzenesulfonamide; 4-Chloro-N-{[3-(4-chloro-phenyl)-4-phenyl-4,5-dihydro-pyrazol-1-yl]-[(pyridin-3-ylmethyl)-amino]-methylene}-benzenesulfonamide; 1-[3-(4-Chloro-phenyl)-4-pheno-4,5-dihydro-pyrazol-1-yl]-3-(1H-indol-2-yl)-2-methylamino-propan-1-one; 2-[3-(4-Chloro-phenyl)-4-phenyl-4,5-dihydro-pyrazol-1-yl]-5-ethyl-4,5-dihydro-oxazole; 4-Chloro-N-[[3-(4-chloro-phenyl)-4-phenyl-4,5-dihydro-pyrazol-1-yl]-(3-hydroxy-2,2-dimethyl-propylamino)-methylene]-benzenesulfonamide; N,N-Diethylamino-1-sulfonic acid [3-(4-chloro-phenyl)-4-hydroxy-4-phenyl-4,5-dihydro-pyrazol-1-yl]-methylamino-methyleneamide; 5-(4-Bromo-phenyl)-1-(2,4-dichloro-phenyl)-1H-pyrazole-3-carbonitrile; 8-Chloro-1-(2,4-dichloro-phenyl)-1,3a,4,5,6,10b-hexahydro-1,2-diaza-benzo[e]azulene-3-carboxylic acid piperidin-1-ylamide; 5-(4-Bromo-phenyl)-1-(2,4-dichloro-phenyl)-3-[2-(3,5-difluoro-phenyl)-2-methanesulfonyl-vinyl]-4-methyl-1H-pyrazole; Piperidine-1-carboxylic acid [5-(4-chloro-phenyl)-1-(2,4-dichloro-phenyl)-4-methyl-1H-pyrazol-3-yl]-amide; 1-(4-Chloro-phenyl)-2-(2,4-dichloro-phenyl)-5-ethylsulfanyl-1H-imidazole-4-carboxylic acid piperidin-1-ylamide; 2-(2,4-Dichloro-phenyl)-1-(4-trifluoromethyl-phenyl)-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid piperidin-1-ylamide; 1-(4-Chloro-phenyl)-2-(2,4-dichloro-phenyl)-1H-imidazole-4-carboxylic acid piperidin-1-ylamide; 1-(4-Chloro-phenyl)-2-(2,4-dichloro-phenyl)-5-ethyl-1H-imidazole-4-carboxylic acid piperidin-1-ylamide; 1-(4-Bromo-phenyl)-2-(2,4-dichloro-phenyl)-5-ethyl-1H-imidazole-4-carboxylic acid piperidin-1-ylamide; 1-(4-Bromo-phenyl)-5-chloro-2-(2,4-dichloro-phenyl)-1H-imidazole-4-carboxylic acid piperidin-1-ylamide; 1-(4-Bromo-phenyl)-2-(2,4-dichloro-phenyl)-5-ethyl-1H-imidazole-4-carboxylic acid cyclohexylamide; 1-(4-Bromo-phenyl)-2-(2,4-dichloro-phenyl)-5-ethyl-1H-imidazole-4-carboxylic acid pentylamide; 4-(4-Chloro-phenyl)-5-(2,4-dichloro-phenyl)-1-methyl-1H-imidazole-2-carboxylic acid cyclohexylamide; 4-(4-Chloro-phenyl)-5-(2,4-dichloro-phenyl)-3-methyl-1H-imidazole-2-carboxylic acid cyclohexylamide; 1-(5-Chloro-pyridin-2-yl)-2-(2,4-dichloro-phenyl)-5-ethyl-1H-imidazole-4-carboxylic acid piperidin-1-ylamide; 1-(4-Chloro-phenyl)-2-(2,4-dichloro-phenyl)-5-methyl-1H-imidazole-4-carboxylic acid (4-hydroxy-cyclo-hexyl)-amide; 1-(4-Chloro-phenyl)-2-(2,4-dichloro-phenyl)-5-methyl-1H-imidazole-4-carboxylic acid azepan-1-ylamide; 2-(2,4-Dichloro-phenyl)-5-ethyl-1-phenyl-1H-imidazole-4-carboxylic acid piperidin-1-ylamide; 2-(1,5-Dimethyl-1H-pyrrol-2-yl)-5-ethyl-1-phenyl-1H-imidazole-4-carboxylic acid cyclohexylamide; 1-(4-Chloro-phenyl)-5-ethyl-2-(3-methyl-pyridin-2-yl)-1H-imidazole-4-carboxylic acid piperidin-1-ylamide; 1-(4-Chloro-phenyl)-2-(2,4-dichloro-phenyl)-5-ethyl-1H-imidazole-4-carboxylic acid piperidin-1-ylamide; 1-(4-Bromo-phenyl)-2-(2,4-dichloro-phenyl)-5-ethyl-1H-imidazole-4-carboxylic acid piperidin-1-ylamide; 1-(4-Bromo-phenyl)-5-chloro-2-(2,4-dichloro-phenyl)-1H-imidazole-4-carboxylic acid piperidin-1-ylamide; 1-(4-Bromo-phenyl)-2-(2,4-dichloro-phenyl)-5-ethyl-1H-imidazole-4-carboxylic acid cyclohexylamide; 1-(4-Bromo-phenyl)-2-(2,4-dichloro-phenyl)-5-ethyl-1H-imidazole-4-carboxylic acid pentylamide; 4-(4-Chloro-phenyl)-5-(2,4-dichloro-phenyl)-1-methyl-1H-imidazole-2-carboxylic acid cyclohexylamide; 4-(4-Chloro-phenyl)-5-(2,4-dichloro-phenyl)-3-methyl-1H-imidazole-2-carboxylic acid cyclohexylamide; 1-(5-Chloro-pyridin-2-yl)-2-(2,4-dichloro-phenyl)-5-ethyl-1H-imidazole-4-carboxylic acid piperidin-1-ylamide; 1-(4-Chloro-phenyl)-2-(2,4-dichloro-phenyl)-5-methyl-1H-imidazole-4-carboxylic acid (4-hydroxy-cyclo-hexyl)-amide; 1-(4-Chloro-phenyl)-2-(2,4-dichloro-phenyl)-5-methyl-1H-imidazole-4-carboxylic acid azepan-1-ylamide; 2-(2,4-Dichloro-phenyl)-5-ethyl-1-phenyl-1H-imidazole-4-carboxylic acid piperidin-1-ylamide; 2-(1,5-Dimethyl-1H-pyrrol-2-yl)-5-ethyl-1-phenyl-1H-imidazole-4-carboxylic acid cyclohexylamide; 1-(4-Chloro-phenyl)-5-ethyl-2-(3-methyl-pyridin-2-yl)-1H-imidazole-4-carboxylic acid piperidin-1-ylamide; 1-(4-Chloro-phenyl)-5-ethyl-2-(3-methyl-pyridin-2-yl)-1H-imidazole-4-carboxylic acid cyclohexylamide; 1-(4-Chloro-phenyl)-2-(2,4-dichloro-phenyl)-5-methyl-1H-imidazole-4-carboxylic acid (4-trifluoromethyl-phenyl)-amide; 2-(2,4-Dichloro-phenyl)-5-methyl-1-pyridin-2-yl-1H-imidazole-4-carboxylic acid piperidin-1-ylamide; 1-(4-Chloro-phenyl)-2-(2,4-dichloro-phenyl)-5-fluoromethyl-1H-imidazole-4-carboxylic acid piperidin-1-ylamide; 1-(4-Chloro-phenyl)-2-(2,4-dichloro-phenyl)-5-hydroxymethyl-1H-imidazole-4-carboxylic acid piperidin-1-ylamide; 1-(4-Chloro-phenyl)-2-(2,4-dichloro-phenyl)-5-methylsulfanyl-1H -imidazole-4-carboxylic acid cyclohexylamide; 1-(4-Chloro-phenyl)-2-(2,4-dichloro-phenyl)-5-methanesulfonyl-1H-imidazole-4-carboxylic acid piperidin-1-ylamide; 1-(4-Chloro-phenyl)-2-(2,4-dichloro-phenyl)-5-methanesulfinyl-1H-imidazole-4-carboxylic acid piperidin-1-ylamide; 5-(4-Chloro-phenyl)-4-(2,5-dichloro-phenyl)-1-methyl-1H-imidazole-2-carboxylic acid piperidin-1-ylamide; 2-(2-Chloro-phenyl)-1-(5-chloro-pyridin-2-yl)-5-ethyl-1H-imidazole-4-carboxylic acid piperidin-1-ylamide; 1-(4-Chloro-phenyl)-2-(2,4-dichloro-phenyl)-5-(2,2,2-trifluoro-ethyl)-1H-imidazole-4-carboxylic acid piperidin-1-ylamide; N-[1-(4-Chloro-phenyl)-2-(2,4-dichloro-phenyl)-5-methyl-1H-imidazol-4-yl]-benzamide; 1-(4-Chloro-phenyl)-2-(2,4-dichloro-phenyl)-5-pyrrolidin-1-ylmethyl-1H-imidazole-4-carboxylic acid piperidin-1-ylamide; 2-[1-(4-Chloro-phenyl)-2-(2,4-dichloro-phenyl)-5-methyl-1H-imidazol-4-yl]-hexan-2-ol; 1-(4-Chloro-phenyl)-2-(2,4-dichloro-phenyl)-5-methyl-4-pentyl-1H-imidazole; 2,5-Dimethyl-1-phenyl-1H-imidazole-4-carboxylic acid adamantan-2-ylamide; 1-(4-Chloro-phenyl)-2-(2-chloro-phenyl)-5-methylsulfanyl-1H-imidazole-4-carboxylic acid piperidin-1-ylamide; 2-(2-Chloro-phenyl)-1-(4-trifluoromethyl-phenyl)-1H-imidazole-4-carboxylic acid piperidin-1-ylamide; 5-(4-Chloro-phenyl)-4-(2,4-dichloro-phenyl)-thiazole-2-carboxylic acid piperidin-1-ylamide; 5-(4-Chloro-phenyl)-1-(2,4-dichloro-phenyl)-1H-[1,2,4]triazole-3-carboxylic acid pyrrolidin-1-ylamide; 1-(4-Chloro-phenyl)-5-(2,4-dichloro-phenyl)-1H-[1,2,4]triazole-3-carboxylic acid piperidin-1-yl-amide; 5-Pentyl-4-phenyl-thiazole-2-carboxylic acid (hexahydro-2,5-methano-pentalen-3a-yl)-amide; 4-Pentyl-5-phenyl-thiazole-2-carboxylic acid (hexahydro-2,5-methano-pentalen-3a-yl)-amide; 1-{(4-Chloro-benzene-sulfonylimino)-[3-(4-chloro-phenyl)-4-phenyl-4,5-dihydro-pyrazol-1-yl]-methyl}-piperidine-4-carboxylic acid amide; 4-Chloro-N-{[3-(4-chloro-phenyl)-4-phenyl-4,5-dihydro-pyrazol-1-yl]-[2-(2-oxo-pyrrolidin-1-yl)-ethylamino]-methylene}-benzenesulfonamide; 4-Chloro-N-[[3-(4-chloro-phenyl)-4-phenyl-4,5-dihydro-pyrazol-1-yl]-(2-cyano-ethylamino)-methylene]-benzene-sulfonamide; 4-Chloro-N-[[3-(4-chloro-phenyl)-4-phenyl-4,5-dihydro-pyrazol-1-yl]-(methoxy-methyl-amino)-methylene]-benzenesulfonamide; 4-Chloro-N-{[3-(4-chloro-phenyl)-4-phenyl-4,5-dihydro-pyrazol-1-yl]-[(piperidin-4-ylmethyl)-amino]-methylene}-benzenesulfonamide; 4-Chloro-N-[[3-(4-chloro-phenyl)-4-phenyl-4,5-dihydro-pyrazol-1-yl]-(piperidin-4-ylamino)-methylene]-benzenesulfonamide; and Morpholine-4-sulfonic acid [3-(4-chloro-phenyl)-4-phenyl-4,5-dihydro-pyrazol-1-yl]-(cyclopropylmethyl-amino)-methyleneamide.

6. The method of claim 1 wherein the CBₓ modulator is selected from the group consisting of: 3-(1,1-dimethyl-butyl)-6,6,9-trimethyl-6a,7,10,10a-tetrahydro-6H-benzo[c]chromene; N-Adamantyl-4-pentyl-5-phenyl-thiazole-2-carboxamide; N-{1,3,3-Trimethyl-endo-(1S)-bicyclo[2.2.1]hept-2-yl}-1-[1-(4-methyl)-benzyl-5-(4-chloro-3-methyl-phenyl)-1H-pyrazol-3-carboxamide; (2-Iodo-5-nitro-phenyl)-[1-(1-methyl-piperidin-2-ylmethyl)-1H-indol-3-yl]-methanone; {4-[4-(1,1-Dimethyl-heptyl)-2,6-dimethoxy-phenyl]-6,6-dimethyl-bicyclo[3.1.1]hept-2-en-2-yl}-methanol; 3-(1,1-Dimethyl-heptyl)-9-hydroxymethyl-6,6-dimethyl-6a,7,10,10a-tetrahydro-6H-enzo[c]chromen-1-ol; Icosa-5,8, 11,14-tetraenoic acid 2-hydroxy-1-hydroxymethyl-ethyl ester; 1-Aziridin-1-yl-henicosa-6,9, 12,15-tetraen-2-one; Noladineether; 4,4,4-Trifluoro-butane-1-sulfinic acid 3-(2-hydroxymethyl-indan-4-yloxy)-phenyl ester, compound with form aldehyde; 7-Methoxy-2-oxo-8-pentyloxy-1,2-dihydro-quinoline-3-carboxylic acid (benzo[1,3]dioxol-5-ylmethyl)-amide; N-(1-{4-[4-Chloro-2-(2-fluoro-benzenesulfonyl)-benzenesulfonyl]-phenyl}-ethyl)-methanesulfonamide; [6-Iodo-2-methyl-1-(2-morpholin-4-yl-ethyl)-2,3-dihydro-1H-indol-3-yl]-(4-methoxy-phenyl)-methanone; 1-(4-Chloro-phenyl)-2-(2-chloro-phenyl)-5-ethyl-1H-imidazole-4-carboxylic acid piperidin-1-ylamide; (2-Methyl-1-propyl-2,3-dihydro-1H-indol-3-yl)-naphthalen-1-yl-methanone; 5-(1,1-Dimethyl-heptyl)-2-[5-hydroxy-2-(3-hydroxy-propyl)-cyclohexyl]-phenol; (2-Methyl-3-morpholin-4-ylmethyl-3,4-dihydro-5-oxa-2a-azacenaphthylen-1-yl)-naphthalen-1-yl-methanone; 5-(4-Chloro-phenyl)-1-(2,4-dichloro-phenyl)-4-methyl-1H-pyrazole-3-carboxylic acid piperidin-1-ylamide; 5-(4-Bromo-phenyl)-1-(2,4-dichloro-phenyl)-4-ethyl-1H-pyrazole-3-carboxylic acid piperidin-1-ylamide; and 1-[Bis-(4-chloro-phenyl)-methyl]-3-[(3,5-difluoro-phenyl)-methanesulfonyl-methylene]-azetidine.

7. The method of claim 1 wherein the CBₓ modulator is selected from the group consisting of: 4-Chloro-N-{[3-(4-chloro-phenyl)-4-phenyl-4,5-dihydro-pyrazol-1-yl]-methylamino-methylene}-benzenesulfonamide; N-{Amino-[3-(4-chloro-phenyl)-4-phenyl-4,5-dihydro-pyrazol-1-yl]-methylene}-4-chloro-benzenesulfonamide; 4-Chloro-N-{[3-(4-chloro-phenyl)-4-pyridin-3-yl-4,5-dihydro-pyrazol-1-yl]-methylamino-methylene}-benzenesulfonamide; 4-Chloro-N-{[3-(4-chloro-phenyl)-4-(3-fluoro-phenyl)-4,5-dihydro-pyrazol-1-yl]-methoxyamino-methylene}-benzenesulfonamide; N-{[3-(4-Chloro-phenyl)-4-(3-fluoro-phenyl)-4,5-dihydro-pyrazol-1-yl]-methylamino-methylene}-N,N-dimethyl-sulfonamide; 5-(4-Chloro-phenyl)-1-(2,4-dichloro-phenyl)-4,5-dihydro-1H-pyrazole-3-carboxylic acid piperidine-1-ylamide; Morpholine-4-sulfonic acid [1-(2,4-dichloro-phenyl)-5-phenyl-4,5-dihydro-1H-pyrazol-3-yl]-methylamino-methyleneamide; N-{Amino-[3-(4-chloro-phenyl)-4-phenyl-4,5-dihydro-pyrazol-1-yl]-methylene}-4-chloro-benzenesulfonamide; 4-Chloro-N-[[3-(4-chloro-phenyl)-4-phenyl-4,5-dihydro-pyrazol-1-yl]-(2-ethylamino-ethylamino)-methylene]-benzenesulfonamide; 4-Chloro-N-{[3-(4-chloro-phenyl)-4-phenyl-4,5-dihydro-pyrazol-1-yl]-[(1-methyl-pyrrolidin-2-ylmethyl)-amino]-methylene}-benzenesulfonamide; 4-Chloro-N-{[3-(4-chloro-phenyl)-4-phenyl-4,5-dihydro-pyrazol-1-yl]-[(pyridin-3-ylmethyl)-amino]-methylene}-benzenesulfonamide; 1-(4-Chloro-phenyl)-2-(2,4-dichloro-phenyl)-5-ethylsulfanyl-1H-imidazole-4-carboxylic acid piperidin-1-ylamide; 2-(2,4-Dichloro-phenyl)-1-(4-trifluoromethyl-phenyl)-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid piperidin-1-ylamide; 1-(4-Chloro-phenyl)-2-(2,4-dichloro-phenyl)-5-ethyl-1H-imidazole-4-carboxylic acid piperidin-1-ylamide; 1-(4-Bromo-phenyl)-2-(2,4-dichloro-phenyl)-5-ethyl-1H-imidazole-4-carboxylic acid piperidin-1-ylamide; 1-(4-Bromo-phenyl)-5-chloro-2-(2,4-dichloro-phenyl)-1H-imidazole-4-carboxylic acid piperidin-1-ylamide; 1-(4-Bromo-phenyl)-2-(2,4-dichloro-phenyl)-5-ethyl-1H-imidazole-4-carboxylic acid pentylamide; 1-(4-Chloro-phenyl)-2-(2,4-dichloro-phenyl)-5-methyl-1H-imidazole-4-carboxylic acid azepan-1-ylamide; 1-(4-Chloro-phenyl)-2-(2,4-dichloro-phenyl)-5-fluoromethyl-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid cyclohexylamide; N-[1-(4-Chloro-phenyl)-2-(2,4-dichloro-phenyl)-5-methyl-1H-imidazol-4-yl]-benzamide; 2-[1-(4-Chloro-phenyl)-2-(2,4-dichloro-phenyl)-5-methyl-1H-imidazol-4-yl]-hexan-2-ol; 1-(4-Chloro-phenyl)-2-(2,4-dichloro-phenyl)-5-methyl-4-pentyl-1H-imidazole; 1-(4-Chloro-phenyl)-2-(2-chloro-phenyl)-5-methylsulfanyl-1H-imidazole-4-carboxylic acid piperidin-1-ylamide; 2-(2-Chloro-phenyl)-1-(4-trifluoromethyl-phenyl)-1H-imidazole-4-carboxylic acid piperidin-1-ylamide; 5-(4-Chloro-phenyl)-4-(2,4-dichloro-phenyl)-thiazole-2-carboxylic acid piperidin-1-ylamide; 1-(4-Chloro-phenyl)-5-(2,4-dichloro-phenyl)-1H-[1,2,4]triazole-3-carboxylic acid piperidin-1-yl-amide; 1-{(4-Chloro-benzene-sulfonylimino)-[3-(4-chloro-phenyl)-4-phenyl-4,5-dihydro-pyrazol-1-yl]-methyl}-piperidine-4-carboxylic acid amide; 4-Chloro-N-{[3-(4-chloro-phenyl)-4-phenyl-4,5-dihydro-pyrazol-1-yl]-[2-(2-oxo-pyrrolidin-1-yl)-ethylamino]-methylene}-benzenesulfonamide; 4-Chloro-N-[[3-(4-chloro-phenyl)-4-phenyl-4,5-dihydro-pyrazol-1-yl]-(2-cyano-ethylamino)-methylene]-benzene-sulfonamide; 4-Chloro-N-[[3-(4-chloro-phenyl)-4-phenyl-4,5-dihydro-pyrazol-1-yl]-(methoxy-methyl-amino)-methylene]-benzenesulfonamide; and Morpholine-4-sulfonic acid [3-(4-chloro-phenyl)-4-phenyl-4,5-dihydro-pyrazol-1-yl]-(cyclopropylmethyl-amino)-methyleneamide.

8. The method of claim 1 wherein the CB₁ agonist or CB₂ agonist is selected from the group consisting of: L759633; L759656; {4-[4-(1,1-dimethyl-heptyl)-2,6-dimethoxy-phenyl]-6,6-dimethyl-bicyclo-[3.1.1]hept-2-en-2-yl}-methanol (HU308); JWH015; (2-iodo-5-nitro-phenyl)-[1-(1-methyl-piperidin-2-ylmethyl)-1H-indol-3-yl]-methanone (AM-1241); 3-(1,1-dimethyl-butyl)-6,6,9-trimethyl-6a,7,10,10a-tetrahydro-6H-benzo[c]-chromene (JWH133); N-adamantantyl-4-pentyl-5-phenyl-thiazole-2-carboxamide; 6,6,9-trimethyl-3-pentyl-6a,7,8,10a-tetrahydro-6H-benzo[c]chromen-1-ol; (bicyclo[2.2.1]hept-2-ylamino)-(5-pentyl-4-phenyl-thiazol-2-yl)-methane; 5-(1,1-dimethyl-heptyl)-2-[5-hydroxy-2-(3-hydroxy-propyl)-cyclohexyl]-phenol (CP-55,940); (2-methyl-3-morpholin-4-ylmethyl-3,4-dihydro-5-oxa-2a-aza-acenaphthylen-1-yl)-naphthalen-1-yl-methanone (WIN-55,212-2); ACEA; ACPA; N-adamantyl-4-pentyl-5-phenyl-thiazole-2-carboxamide; methanandamide; anandamide; 2-arachidonoyl glycerol; 2-icosa-5,8,11,14-tetraenyloxy-propane-1,3-diol (noladin ether); BAY 38-7271; SAB-378; BAY 59-3074; O-1057; GW-1000; PRS-211375; PRS-211359; PRS-211355; PRS-211096; PXS-2076; AM-577; GW-842166X; and mixtures thereof.

9. The method of claim 1 wherein the CB₂ agonist is a selective CB₂ agonist and is selected from the group consisting of: 3-(1,1-dimethyl-butyl)-6,6,9-trimethyl-6a,7,10,10a-tetrahydro-6H-benzo[c]chromene (JWH 133); L759633; L759656; {4-[4-(1,1-dimethyl-heptyl)-2,6-dimethoxy-phenyl]-6,6-dimethyl-bicyclo[3.1.1]hept-2-en-2-yl}-methanol (HU308); JWH015; (2-iodo-5-nitro-phenyl)-[1-(1-methyl-piperidin-2-ylmethyl)-1H-indol-3-yl]-methanone (AM-1241); and mixtures thereof.

10. The method of claim 1 wherein the CB₂ antagonist or the CB₂ inverse agonist is selected from the group consisting of: N-{1,3,3-Trimethyl-endo-(1S)-bicyclo[2.2.1]hept-2-yl}-1-(4-methyl)-benzyl-5-(4-chloro-3-methyl-phenyl)-1H-pyrazol-3-carboxamide (SR-144528), JTE-907, AM630, and mixtures thereof.

11. The method of claim 1 wherein the compound having both CBₓ agonist and CB₂ agonist properties is selected from the group consisting of: 2-icosa-5,8,11,14-tetraenyloxy-propane-1, 3-diol.

12. The method of claim 1 wherein the CBₓ modulator is selected from the group consisting of: 6,6,9-trimethyl-3-pentyl-6a,7,8,10a-tetrahydro-6H-benzo[c]chromen-1-ol; N-(Endo-bicyclo[2.2.1]hept-2-yl)-5-pentyl-4-phenyl-thiazole-2-carboxamide; 3-(1,1-dimethyl-butyl)-6,6,9-trimethyl-6a,7,10,10a-tetrahydro-6H-benzo[c]chromene; N-adamantyl-4-pentyl-5-phenyl-thiazole-2-carboxamide; 1-[1-Benzyl-5-(4-chloro-3-methyl-phenyl)-1H-pyrazol-3-yl]-ethanonyl-1,3,3-trimethyl-bi-cyclo[2.2.1]hept-2-ylamine; (2-iodo-5-nitro-phenyl)-[1-(1-methyl-piperidin-2-ylmethyl)-1H-indol-3-yl]-methanone; {4-[4-(1,1-Dimethyl-heptyl)-2,6-dimethoxy-phenyl]-6,6-dimethyl-bicyclo[3.1.1]hept-2-en-2-yl}-methanol; and mixtures thereof.

13. A method of treating a medical condition in a mammal comprising:
administering to a subject in need thereof a pharmaceutically effective amount of a CBₓ modulator;
wherein the CBₓ modulator is selected from the group consisting of: CB₁ agonists, CB₂ agonists, CB₂ partial agonists, CB₂ antagonists, CB₂ inverse agonists, compounds having both CB₁ agonist and CB₂ agonist properties, and mixtures thereof; and
wherein the medical condition is selected from the group consisting of obesity, diabetes mellitus, metabolic syndrome, syndrome X, insulinoma, familial hyperinsulemic hypoglycemia, male pattern baldness, detrusor hyperreactivity, asthma, neuroprotection, epilepsy, analgesia, cardioprotection, angina, cardioplegia, arrhythmia, coronary spasm, peripheral vascular disease, cerebral vasospasm, appetite regulation, neurodegeneration, pain, neuropathic pain, chronic pain, idiopathic pain and impotence.

14. The method of claim 13 wherein the metabolic syndrome or syndrome X comprise disorders or diseases selected from the group consisting of: hypertension, insulin resistance, glucose intolerance and dyslipoproteinaemia.

15. The method of claim 13 wherein the CB_{X} modulator is a modulator of at least one channel selected from the group consisting of: Kir6.2/SUR1K_{ATP}, Kir6.2/SUR2B K_{ATP}, Kir6.1/SUR2B K_{ATP}, and Kir6.2/SUR2A K_{ATP}.

16. The method of claim 13 wherein the K_{ATP} channel modulator is a K_{ATP} channel opener.

17. The method of claim 13 wherein the CBₓ modulator is selected from the group consisting of: 3-(1,1-dimethyl-butyl)-6,6,9-trimethyl-6a,7,10,10a-tetrahydro-6H-benzo[c]chromene; N-Adamantyl-4-pentyl-5-phenyl-thiazole-2-carboxamide; N-{1,3,3-Trimethyl-endo-(1S)-bicyclo[2.2.1]hept-2-yl}-1-[1-(4-methyl)-benzyl-5-(4-chloro-3-methyl-phenyl)-1H-pyrazol-3-carboxamide; (2-Iodo-5-nitro-phenyl)-[1-(1-methyl-piperidin-2-ylmethyl)-1H-indol-3-yl]-methanone; {4-[4-(1,1-Dimethyl-heptyl)-2,6-dimethoxy-phenyl]-6,6-dimethyl-bicyclo[3.1.1]hept-2-en-2-yl}-methanol; 3-(1,1-Dimethyl-heptyl)-9-hydroxymethyl-6,6-dimethyl-6a,7,10,10a-tetrahydro-6H-enzo[c]chromen-1-ol; Icosa-5,8, 11,14-tetraenoic acid 2-hydroxy-1-hydroxymethyl-ethyl ester; 1-Aziridin-1-yl-henicosa-6,9,12,15-tetraen-2-one; Noladineether; 4,4,4-Trifluoro-butane-1-sulfinic acid 3-(2-hydroxymethyl-indan-4-yloxy)-phenyl ester, compound with form aldehyde; 7-Methoxy-2-oxo-8-pentyloxy-1,2-dihydro-quinoline-3-carboxylic acid (benzo[1,3]dioxol-5-ylmethyl)-amide; N-(1-{4-[4-Chloro-2-(2-fluoro-benzenesulfonyl)-benzenesulfonyl]-phenyl}-ethyl)-methanesulfonamide; [6-Iodo-2-methyl-1-(2-morpholin-4-yl-ethyl)-2,3-dihydro-1H-indol-3-yl]-(4-methoxy-phenyl)-methanone; 1-(4-Chloro-phenyl)-2-(2-chloro-phenyl)-5-ethyl-1H-imidazole-4-carboxylic acid piperidin-1-ylamide; (2-Methyl-1-propyl-2,3-dihydro-1H-indol-3-yl)-naphthalen-1-yl-methanone; 5-(1,1-Dimethyl-heptyl)-2-[5-hydroxy-2-(3-hydroxy-propyl)-cyclohexyl]-phenol; (2-Methyl-3-morpholin-4-ylmethyl-3,4-dihydro-5-oxa-2a-azacenaphthylen-1-yl)-naphthalen-1-yl-methanone; 5-(4-Chloro-phenyl)-1-(2,4-dichloro-phenyl)-4-methyl-1H-pyrazole-3-carboxylic acid piperidin-1-ylamide; 5-(4-Bromo-phenyl)-1-(2,4-dichloro-phenyl)-4-ethyl-1H-pyrazole-3-carboxylic acid piperidin-1-ylamide; 1-[Bis-(4-chloro-phenyl)-methyl]-3-[(3,5-difluoro-phenyl)-methanesulfonyl-methylene]-azetidine; 4-Chloro-N-{[3-(4-chloro-phenyl)-4-phenyl-4,5-dihydro-pyrazol-1-yl]-methylamino-methylene}-benzenesulfonamide; N-{Amino-[3-(4-chloro-phenyl)-4-phenyl-4,5-dihydro-pyrazol-1-yl]-methylene}-4-chloro-benzenesulfonamide; N-{[3-(4-Chloro-phenyl)-4-pyridin-3-yl-4,5-dihydro-pyrazol-1-yl]-methylamino-methylene}-4-trifluoromethyl-benzenesulfonamide; 4-Chloro-N-{[3-(4-chloro-phenyl)-4-pyridin-3-yl-4,5-dihydro-pyrazol-1-yl]-methylamino-methylene}-benzenesulfonamide; 4-Chloro-N-{[3-(4-chloro-phenyl)-4-(3-fluoro-phenyl)-4,5-dihydro-pyrazol-1-yl]-methoxyamino-methylene}-benzenesulfonamide; Morpholine-4-sulfonic acid [3-(4-chloro-phenyl)-4-phenyl-4,5-dihydro-pyrazol-1-yl]-methylamino-methyleneamide; N-{[3-(4-Chloro-phenyl)-4-(3-fluoro-phenyl)-4,5-dihydro-pyrazol-1-yl]-methylamino-methylene}-N,N-dimethyl-sulfonamide; Azepane-1-sulfonic acid [3-(4-chloro-phenyl)-4-phenyl-4,5-dihydro-pyrazol-1-yl]-methylamino-methyleneamide; 4-Chloro-N-{[3-(4-chloro-phenyl)-4-phenyl-4,5-dihydro-pyrazol-1-yl]-[(1-methyl-pyrrolidin-3-ylmethyl)-amino]-methylene}-benzenesulfonamide; 1-(4-Chloro-phenyl)-5-phenyl-4,5-dihydro-1H-pyrazole-3-carboxamidine; N-{[3-(4-Chloro-phenyl)-4-phenyl-4,5-dihydro-pyrazol-1-yl]-methylamino-methylene}-4-trifluoromethyl-benzene-sulfonamide; Piperidine-1-sulfonic acid [3-(4-chloro-phenyl)-4-phenyl-4,5-dihydro-pyrazol-1-yl]-methylamino-methyleneamide; Piperidine-1-sulfonic acid [3-(4-chloro-phenyl)-4-phenyl-4,5-dihydro-pyrazol-1-yl]-(2-dimethylamino-ethylamino)-methyleneamide; N,N-Diethylamino-1-sulfonic acid [3-(4-chloro-phenyl)-4-phenyl-4,5-dihydro-pyrazol-1-yl]-methylsulfanyl-methyleneamide; 2-Amino-1-[3-(4-chloro-phenyl)-4-phenyl-4,5-dihydro-pyrazol-1-yl]-3-(3,4-dichloro-phenyl)-propan-1-one; Morpholine-4-sulfonic acid [3-(4-chloro-phenyl)-4-phenyl-4,5-dihydro-pyrazol-1-yl]-methylamino-methyleneamide; N,N-Dimethylamino-1-sulfonic acid [3-(4-chloro-phenyl)-4-phenyl-4,5-dihydro-pyrazol-1-yl]-(2-fluoro-ethylamino)-methyleneamide; Piperidine-1-sulfonic acid [3-(4-chloro-phenyl)-4-(3-fluoro-phenyl)-4,5-dihydro-pyrazol-1-yl]-methylamino-methyleneamide; 5-(4-Chloro-phenyl)-1-(2,4-dichloro-phenyl)-4,5-dihydro-1H-pyrazole-3-carboxylic acid piperidine-1-ylamide; 1-(4-Chloro-phenyl)-5-phenyl-4,5-dihydro-1H-pyrazole-3-carboxylic acid piperidin-1-ylamide; Piperidine-1-sulfonic acid [1-(4-chloro-phenyl)-5-phenyl-4,5-dihydro-1H-pyrazol-3-yl]-methylamino-methyleneamide; Morpholine-4-sulfonic acid [1-(2,4-dichloro-phenyl)-5-phenyl-4,5-dihydro-1H-pyrazol-3-yl]-methylamino-methyleneamide; 4-Chloro-N-[[3-(4-chloro-phenyl)-4-phenyl-4,5-dihydro-pyrazol-1-yl]-(2-fluoro-ethylamino)-methylene]-benzenesulfonamide; 4-Chloro-N-[[3-(4-chloro-phenyl)-4-phenyl-4,5-dihydro-pyrazol-1-yl]-(2-fluoro-ethylamino)-methylene]-benzenesulfonamide; N-{Amino-[3-(4-chloro-phenyl)-4-phenyl-4,5-dihydro-pyrazol-1-yl]-methylene}-4-chloro-benzenesulfonamide; 4-Chloro-N-[3-(4-chloro-phenyl)-4-phenyl-4,5-dihydro-pyrazole-1-carbonyl]-benzenesulfonamide; 4-Chloro-N-[[3-(4-chloro-phenyl)-4-phenyl-4,5-dihydro-pyrazol-1-yl]-(2-ethylamino-ethylamino)-methylene]-benzenesulfonamide; 4-Chloro-N-{[3-(4-chloro-phenyl)-4-phenyl-4,5-dihydro-pyrazol-1-yl]-[(1-methyl-pyrrolidin-2-ylmethyl)-amino]-methylene}-benzenesulfonamide; 4-Chloro-N-[[3-(4-chloro-phenyl)-4-phenyl-4,5-dihydro-pyrazol-1-yl]-(4-pyrrolidin-1-yl-butylamino)-methylene]-benzenesulfonamide; 4-Chloro-N-{[3-(4-chloro-phenyl)-4-phenyl-4,5-dihydro-pyrazol-1-yl]-[(pyridin-3-ylmethyl)-amino]-methylene}-benzenesulfonamide; 1-[3-(4-Chloro-phenyl)-4-phenyl-4,5-dihydro-pyrazol-1-yl]-3-(1H-indol-2-yl)-2-methylamino-propan-1-one; 2-[3-(4-Chloro-phenyl)-4-phenyl-4,5-dihydro-pyrazol-1-yl]-5-ethyl-4,5-dihydro-oxazole; 4-Chloro-N-[[3-(4-chloro-phenyl)-4-phenyl-4,5-dihydro-pyrazol-1-yl]-(3-hydroxy-2,2-dimethyl-propylamino)-methylene]-benzenesulfonamide; N,N-Diethylamino-1-sulfonic acid [3-(4-chloro-phenyl)-4-hydroxy-4-phenyl-4,5-dihydro-pyrazol-1-yl]-methylamino-methyleneamide; 5-(4-Bromo-phenyl)-1-(2,4-dichloro-phenyl)-1H-pyrazole-3-carbonitrile; 8-Chloro-1-(2,4-dichloro-phenyl)-1,3a,4,5,6,10b-hexahydro-1,2-diaza-benzo[e]azulene-3-carboxylic acid piperidin-1-ylamide; 5-(4-Bromo-phenyl)-1-(2,4-dichloro-phenyl)-3-[2-(3,5-difluoro-phenyl)-2-methanesulfonyl-vinyl]-4-methyl-1H-pyrazole; Piperidine-1-carboxylic acid [5-(4-chloro-phenyl)-1-(2,4-dichloro-phenyl)-4-methyl-1H-pyrazol-3-yl]-amide; 1-(4-Chloro-phenyl)-2-(2,4-dichloro-phenyl)-5-ethylsulfanyl-1H-imidazole-4-carboxylic acid piperidin-1-ylamide; 2-(2,4-Dichloro-phenyl)-1-(4-trifluoromethyl-phenyl)-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid piperidin-1-ylamide; 1-(4-Chloro-phenyl)-2-(2,4-dichloro-phenyl)-1H-imidazole-4-carboxylic acid piperidin-1-ylamide; 1-(4-Chloro-phenyl)-2-(2,4-dichloro-phenyl)-5-ethyl-1H-imidazole-4-carboxylic acid piperidin-1-ylamide; 1-(4-Bromo-phenyl)-2-(2,4-dichloro-phenyl)-5-ethyl-1H-imidazole-4-carboxylic acid piperidin-1-ylamide; 1-(4-Bromo-phenyl)-5-chloro-2-(2,4-dichloro-phenyl)-1H-imidazole-4-carboxylic acid piperidin-1-ylamide; 1-(4-Bromo-phenyl)-2-(2,4-dichloro-phenyl)-5-ethyl-1H-imidazole-4-carboxylic acid cyclohexylamide; 1-(4-Bromo-phenyl)-2-(2,4-dichloro-phenyl)-5-ethyl-1H-imidazole-4-carboxylic acid pentylamide; 4-(4-Chloro-phenyl)-5-(2,4-dichloro-phenyl)-1-methyl-1H-imidazole-2-carboxylic acid cyclohexylamide; 4-(4-Chloro-phenyl)-5-(2,4-dichloro-phenyl)-3-methyl-1H-imidazole-2-carboxylic acid cyclohexylamide; 1-(5-Chloro-pyridin-2-yl)-2-(2,4-dichloro-phenyl)-5-ethyl-1H-imidazole-4-carboxylic acid piperidin-1-ylamide; 1-(4-Chloro-phenyl)-2-(2,4-dichloro-phenyl)-5-methyl-1H-imidazole-4-carboxylic acid (4-hydroxy-cyclo-hexyl)-amide; 1-(4-Chloro-phenyl)-2-(2,4-dichloro-phenyl)-5-methyl-1H-imidazole-4-carboxylic acid azepan-1-ylamide; 2-(2,4-Dichloro-phenyl)-5-ethyl-1-phenyl-1H-imidazole-4-carboxylic acid piperidin-1-ylamide; 2-(1,5-Dimethyl-1H-pyrrol-2-yl)-5-ethyl-1-phenyl-1H-imidazole-4-carboxylic acid cyclohexylamide; 1-(4-Chloro-phenyl)-5-ethyl-2-(3-methyl-pyridin-2-yl)-1H-imidazole-4-carboxylic acid piperidin-1-ylamide; 1-(4-Chloro-phenyl)-2-(2,4-dichloro-phenyl)-5-ethyl-1H-imidazole-4-carboxylic acid piperidin-1-ylamide; 1-(4-Bromo-phenyl)-2-(2,4-dichloro-phenyl)-5-ethyl-1H-imidazole-4-carboxylic acid piperidin-1-ylamide; 1-(4-Bromo-phenyl)-5-chloro-2-(2,4-dichloro-phenyl)-1H-imidazole-4-carboxylic acid piperidin-1-ylamide; 1-(4-Bromo-phenyl)-2-(2,4-dichloro-phenyl)-5-ethyl-1H-imidazole-4-carboxylic acid cyclohexylamide; 1-(4-Bromo-phenyl)-2-(2,4-dichloro-phenyl)-5-ethyl-1H-imidazole-4-carboxylic acid pentylamide; 4-(4-Chloro-phenyl)-5-(2,4-dichloro-phenyl)-1-methyl-1H-imidazole-2-carboxylic acid cyclohexylamide; 4-(4-Chloro-phenyl)-5-(2,4-dichloro-phenyl)-3-methyl-1H-imidazole-2-carboxylic acid cyclohexylamide; 1-(5-Chloro-pyridin-2-yl)-2-(2,4-dichloro-phenyl)-5-ethyl-1H-imidazole-4-carboxylic acid piperidin-1-ylamide; 1-(4-Chloro-phenyl)-2-(2,4-dichloro-phenyl)-5-methyl-1H-imidazole-4-carboxylic acid (4-hydroxy-cyclo-hexyl)-amide; 1-(4-Chloro-phenyl)-2-(2,4-dichloro-phenyl)-5-methyl-1H-imidazole-4-carboxylic acid azepan-1-ylamide; 2-(2,4-Dichloro-phenyl)-5-ethyl-1-phenyl-1H-imidazole-4-carboxylic acid piperidin-1-ylamide; 2-(1,5-Dimethyl-1H-pyrrol-2-yl)-5-ethyl-1-phenyl-1H-imidazole-4-carboxylic acid cyclohexylamide; 1-(4-Chloro-phenyl)-5-ethyl-2-(3-methyl-pyridin-2-yl)-1H-imidazole-4-carboxylic acid piperidin-1-ylamide; 1-(4-Chloro-phenyl)-5-ethyl-2-(3-methyl-pyridin-2-yl)-1H-imidazole-4-carboxylic acid cyclohexylamide; 1-(4-Chloro-phenyl)-2-(2,4-dichloro-phenyl)-5-methyl-1H-imidazole-4-carboxylic acid (4-trifluoromethyl-phenyl)-amide; 2-(2,4-Dichloro-phenyl)-5-methyl-1-pyridin-2-yl-1H-imidazole-4-carboxylic acid piperidin-1-ylamide; 1-(4-Chloro-phenyl)-2-(2,4-dichloro-phenyl)-5-fluoromethyl-1H-imidazole-4-carboxylic acid piperidin-1-ylamide; 1-(4-Chloro-phenyl)-2-(2,4-dichloro-phenyl)-5-hydroxymethyl-1H-imidazole-4-carboxylic acid piperidin-1-ylamide; 1-(4-Chloro-phenyl)-2-(2,4-dichloro-phenyl)-5-methylsulfanyl-1H -imidazole-4-carboxylic acid cyclohexylamide; 1-(4-Chloro-phenyl)-2-(2,4-dichloro-phenyl)-5-methanesulfonyl-1H-imidazole-4-carboxylic acid piperidin-1-ylamide; 1-(4-Chloro-phenyl)-2-(2,4-dichloro-phenyl)-5-methanesulfinyl-1H-imidazole-4-carboxylic acid piperidin-1-ylamide; 5-(4-Chloro-phenyl)-4-(2,5-dichloro-phenyl)-1-methyl-1H-imidazole-2-carboxylic acid piperidin-1-ylamide; 2-(2-Chloro-phenyl)-1-(5-chloro-pyridin-2-yl)-5-ethyl-1H-imidazole-4-carboxylic acid piperidin-1-ylamide; 1-(4-Chloro-phenyl)-2-(2,4-dichloro-phenyl)-5-(2,2,2-trifluoro-ethyl)-1H-imidazole-4-carboxylic acid piperidin-1-ylamide; N-[1-(4-Chloro-phenyl)-2-(2,4-dichloro-phenyl)-5-methyl-1H-imidazol-4-yl]-benzamide; 1-(4-Chloro-phenyl)-2-(2,4-dichloro-phenyl)-5-pyrrolidin-1-ylmethyl-1H-imidazole-4-carboxylic acid piperidin-1-ylamide; 2-[1-(4-Chloro-phenyl)-2-(2,4-dichloro-phenyl)-5-methyl-1H-imidazol-4-yl]-hexan-2-ol; 1-(4-Chloro-phenyl)-2-(2,4-dichloro-phenyl)-5-methyl-4-pentyl-1H-imidazole; 2,5-Dimethyl-1-phenyl-1H-imidazole-4-carboxylic acid adamantan-2-ylamide; 1-(4-Chloro-phenyl)-2-(2-chloro-phenyl)-5-methylsulfanyl-1H-imidazole-4-carboxylic acid piperidin-1-ylamide; 2-(2-Chloro-phenyl)-1-(4-trifluoromethyl-phenyl)-1H-imidazole-4-carboxylic acid piperidin-1-ylamide; 5-(4-Chloro-phenyl)-4-(2,4-dichloro-phenyl)-thiazole-2-carboxylic acid piperidin-1-ylamide; 5-(4-Chloro-phenyl)-1-(2,4-dichloro-phenyl)-1H-[1,2,4]triazole-3-carboxylic acid pyrrolidin-1-ylamide; 1-(4-Chloro-phenyl)-5-(2,4-dichloro-phenyl)-1H-[1,2,4]triazole-3-carboxylic acid piperidin-1-yl-amide; 5-Pentyl-4-phenyl-thiazole-2-carboxylic acid (hexahydro-2,5-methano-pentalen-3a-yl)-amide; 4-Pentyl-5-phenyl-thiazole-2-carboxylic acid (hexahydro-2,5-methano-pentalen-3a-yl)-amide; 1-{(4-Chloro-benzene-sulfonylimino)-[3-(4-chloro-phenyl)-4-phenyl-4,5-dihydro-pyrazol-1-yl]-methyl}-piperidine-4-carboxylic acid amide; 4-Chloro-N-{[3-(4-chloro-phenyl)-4-phenyl-4,5-dihydro-pyrazol-1-yl]-[2-(2-oxo-pyrrolidin-1-yl)-ethylamino]-methylene}-benzenesulfonamide; 4-Chloro-N-[[3-(4-chloro-phenyl)-4-phenyl-4,5-dihydro-pyrazol-1-yl]-(2-cyano-ethylamino)-methylene]-benzene-sulfonamide; 4-Chloro-N-[[3-(4-chloro-phenyl)-4-phenyl-4,5-dihydro-pyrazol-1-yl]-(methoxy-methyl-amino)-methylene]-benzenesulfonamide; 4-Chloro-N-{[3-(4-chloro-phenyl)-4-phenyl-4,5-dihydro-pyrazol-1-yl]-[(piperidin-4-ylmethyl)-amino]-methylene}-benzenesulfonamide; 4-Chloro-N-[[3-(4-chloro-phenyl)-4-phenyl-4,5-dihydro-pyrazol-1-yl]-(piperidin-4-ylamino)-methylene]-benzenesulfonamide; and Morpholine-4-sulfonic acid [3-(4-chloro-phenyl)-4-phenyl-4,5-dihydro-pyrazol-1-yl]-(cyclopropylmethyl-amino)-methyleneamide.

18. The method of claim 13 wherein the CBₓ modulator is selected from the group consisting of: 3-(1,1-dimethyl-butyl)-6,6,9-trimethyl-6a,7,10,10a-tetrahydro-6H-benzo[c]chromene; N-Adamantyl-4-pentyl-5-phenyl-thiazole-2-carboxamide; N-{1,3,3-Trimethyl-endo-(1S)-bicyclo[2.2.1]hept-2-yl}-1-[1-(4-methyl)-benzyl-5-(4-chloro-3-methyl-phenyl)-1H-pyrazol-3-carboxamide; (2-Iodo-5-nitro-phenyl)-[1-(1-methyl-piperidin-2-ylmethyl)-1H-indol-3-yl]-methanone; {4-[4-(1,1-Dimethyl-heptyl)-2,6-dimethoxy-phenyl]-6,6-dimethyl-bicyclo[3.1.1]hept-2-en-2-yl}-methanol; 3-(1,1-Dimethyl-heptyl)-9-hydroxymethyl-6,6-dimethyl-6a,7,10,10a-tetrahydro-6H-enzo[c]chromen-1-ol; Icosa-5,8, 11,14-tetraenoic acid 2-hydroxy-1-hydroxymethyl-ethyl ester; 1-Aziridin-1-yl-henicosa-6,9, 12,15-tetraen-2-one; Noladineether; 4,4,4-Trifluoro-butane-1-sulfinic acid 3-(2-hydroxymethyl-indan-4-yloxy)-phenyl ester, compound with form aldehyde; 7-Methoxy-2-oxo-8-pentyloxy-1,2-dihydro-quinoline-3-carboxylic acid (benzo[1,3]dioxol-5-ylmethyl)-amide; N-(1-{4-[4-Chloro-2-(2-fluoro-benzenesulfonyl)-benzenesulfonyl]-phenyl}-ethyl)-methanesulfonamide; [6-Iodo-2-methyl-1-(2-morpholin-4-yl-ethyl)-2,3-dihydro-1H-indol-3-yl]-(4-methoxy-phenyl)-methanone; 1-(4-Chloro-phenyl)-2-(2-chloro-phenyl)-5-ethyl-1H-imidazole-4-carboxylic acid piperidin-1-ylamide; (2-Methyl-1-propyl-2,3-dihydro-1H-indol-3-yl)-naphthalen-1-yl-methanone; 5-(1,1-Dimethyl-heptyl)-2-[5-hydroxy-2-(3-hydroxy-propyl)-cyclohexyl]-phenol; (2-Methyl-3-morpholin-4-ylmethyl-3,4-dihydro-5-oxa-2a-azacenaphthylen-1-yl)-naphthalen-1-yl-methanone; 5-(4-Chloro-phenyl)-1-(2,4-dichloro-phenyl)-4-methyl-1H-pyrazole-3-carboxylic acid piperidin-1-ylamide; 5-(4-Bromo-phenyl)-1-(2,4-dichloro-phenyl)-4-ethyl-1H-pyrazole-3-carboxylic acid piperidin-1-ylamide; and 1-[Bis-(4-chloro-phenyl)-methyl]-3-[(3,5-difluoro-phenyl)-methanesulfonyl-methylene]-azetidine.

19. The method of claim 13 wherein the CBₓ modulator is selected from the group consisting of: 4-Chloro-N-{[3-(4-chloro-phenyl)-4-phenyl-4,5-dihydro-pyrazol-1-yl]-methylamino-methylene}-benzenesulfonamide; N-{Amino-[3-(4-chloro-phenyl)-4-phenyl-4,5-dihydro-pyrazol-1-yl]-methylene}-4-chloro-benzenesulfonamide; 4-Chloro-N-{[3-(4-chloro-phenyl)-4-pyridin-3-yl-4,5-dihydro-pyrazol-1-yl]-methylamino-methylene}-benzenesulfonamide; 4-Chloro-N-{[3-(4-chloro-phenyl)-4-(3-fluoro-phenyl)-4,5-dihydro-pyrazol-1-yl]-methoxyamino-methylene}-benzenesulfonamide; N-{[3-(4-Chloro-phenyl)-4-(3-fluoro-phenyl)-4,5-dihydro-pyrazol-1-yl]-methylamino-methylene}-N,N-dimethyl-sulfonamide; 5-(4-Chloro-phenyl)-1-(2,4-dichloro-phenyl)-4,5-dihydro-1H-pyrazole-3-carboxylic acid piperidine-1-ylamide; Morpholine-4-sulfonic acid [1-(2,4-dichloro-phenyl)-5-phenyl-4,5-dihydro-1H-pyrazol-3-yl]-methylamino-methyleneamide; N-{Amino-[3-(4-chloro-phenyl)-4-phenyl-4,5-dihydro-pyrazol-1-yl]-methylene}-4-chloro-benzenesulfonamide; 4-Chloro-N-[[3-(4-chloro-phenyl)-4-phenyl-4,5-dihydro-pyrazol-1-yl]-(2-ethylamino-ethylamino)-methylene]-benzenesulfonamide; 4-Chloro-N-{[3-(4-chloro-phenyl)-4-phenyl-4,5-dihydro-pyrazol-1-yl]-[(1-methyl-pyrrolidin-2-ylmethyl)-amino]-methylene}-benzenesulfonamide; 4-Chloro-N-{[3-(4-chloro-phenyl)-4-phenyl-4,5-dihydro-pyrazol-1-yl]-[(pyridin-3-ylmethyl)-amino]-methylene}-benzenesulfonamide; 1-(4-Chloro-phenyl)-2-(2,4-dichloro-phenyl)-5-ethylsulfanyl-1H-imidazole-4-carboxylic acid piperidin-1-ylamide; 2-(2,4-Dichloro-phenyl)-1-(4-trifluoromethyl-phenyl)-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid piperidin-1-ylamide; 1-(4-Chloro-phenyl)-2-(2,4-dichloro-phenyl)-5-ethyl-1H-imidazole-4-carboxylic acid piperidin-1-ylamide; 1-(4-Bromo-phenyl)-2-(2,4-dichloro-phenyl)-5-ethyl-1H-imidazole-4-carboxylic acid piperidin-1-ylamide; 1-(4-Bromo-phenyl)-5-chloro-2-(2,4-dichloro-phenyl)-1H-imidazole-4-carboxylic acid piperidin-1-ylamide; 1-(4-Bromo-phenyl)-2-(2,4-dichloro-phenyl)-5-ethyl-1H-imidazole-4-carboxylic acid pentylamide; 1-(4-Chloro-phenyl)-2-(2,4-dichloro-phenyl)-5-methyl-1H-imidazole-4-carboxylic acid azepan-1-ylamide; 1-(4-Chloro-phenyl)-2-(2,4-dichloro-phenyl)-5-fluoromethyl-1H-imidazole-4-carboxylic acid piperidin-1-ylamide; 1-(4-Chloro-phenyl)-2-(2,4-dichloro-phenyl)-5-methylsulfanyl-1H-imidazole-4-carboxylic acid cyclohexylamide; N-[1-(4-Chloro-phenyl)-2-(2,4-dichloro-phenyl)-5-methyl-1H-imidazol-4-yl]-benzamide; 2-[1-(4-Chloro-phenyl)-2-(2,4-dichloro-phenyl)-5-methyl-1H-imidazol-4-yl]-hexan-2-ol; 1-(4-Chloro-phenyl)-2-(2,4-dichloro-phenyl)-5-methyl-4-pentyl-1H-imidazole; 1-(4-Chloro-phenyl)-2-(2-chloro-phenyl)-5-methylsulfanyl-1H-imidazole-4-carboxylic acid piperidin-1-ylamide; 2-(2-Chloro-phenyl)-1-(4-trifluoromethyl-phenyl)-1H-imidazole-4-carboxylic acid piperidin-1-ylamide; 5-(4-Chloro-phenyl)-4-(2,4-dichloro-phenyl)-thiazole-2-carboxylic acid piperidin-1-ylamide; 1-(4-Chloro-phenyl)-5-(2,4-dichloro-phenyl)-1H-[1,2,4]triazole-3-carboxylic acid piperidin-1-yl-amide; 1-{(4-Chloro-benzene-sulfonylimino)-[3-(4-chloro-phenyl)-4-phenyl-4,5-dihydro-pyrazol-1-yl]-methyl}-piperidine-4-carboxylic acid amide; 4-Chloro-N-{[3-(4-chloro-phenyl)-4-phenyl-4,5-dihydro-pyrazol-1-yl]-[2-(2-oxo-pyrrolidin-1-yl)-ethylamino]-methylene}-benzenesulfonamide; 4-Chloro-N-[[3-(4-chloro-phenyl)-4-phenyl-4,5-dihydro-pyrazol-1-yl]-(2-cyano-ethylamino)-methylene]-benzene-sulfonamide; 4-Chloro-N-[[3-(4-chloro-phenyl)-4-phenyl-4,5-dihydro-pyrazol-1-yl]-(methoxy-methyl-amino)-methylene]-benzenesulfonamide; and Morpholine-4-sulfonic acid [3-(4-chloro-phenyl)-4-phenyl-4,5-dihydro-pyrazol-1-yl]-(cyclopropylmethyl-amino)-methyleneamide.

20. The method of claim 13 wherein the CB₁ agonist or CB₂ agonist is selected from the group consisting of: L759633; L759656; {4-[4-(1,1-dimethyl-heptyl)-2,6-dimethoxy-phenyl]-6,6-dimethyl-bicyclo-[3.1.1]hept-2-en-2-yl}-methanol (HU308); JWH015; (2-iodo-5-nitro-phenyl)-[1-(1-methyl-piperidin-2-ylmethyl)-1H-indol-3-yl]-methanone (AM-1241); 3-(1,1-dimethyl-butyl)-6,6,9-trimethyl-6a,7,10,10a-tetrahydro-6H-benzo[c]-chromene (JWH133); N-adamantantyl-4-pentyl-5-phenyl-thiazole-2-carboxamide; 6,6,9-trimethyl-3-pentyl-6a,7,8,10a-tetrahydro-6H-benzo[c]chromen-1-ol; (bicyclo[2.2.1]hept-2-ylamino)-(5-pentyl-4-phenyl-thiazol-2-yl)-methane; 5-(1,1-dimethyl-heptyl)-2-[5-hydroxy-2-(3-hydroxy-propyl)-cyclohexyl]-phenol (CP-55,940); (2-methyl-3-morpholin-4-ylmethyl-3,4-dihydro-5-oxa-2a-aza-acenaphthylen-1-yl)-naphthalen-1-yl-methanone (WIN-55,212-2); ACEA; ACPA; N-adamantyl-4-pentyl-5-phenyl-thiazole-2-carboxamide; methanandamide; anandamide; 2-arachidonoyl glycerol; 2-icosa-5,8,11,14-tetraenyloxy-propane-1,3-diol (noladin ether); BAY 38-7271; SAB-378; BAY 59-3074; O-1057; GW-1000; PRS-211375; PRS-211359; PRS-211355; PRS-211096; PXS-2076; AM-577; GW-842166X; and mixtures thereof.

21. The method of claim 13 wherein the CB₂ agonist is a selective CB₂ agonist and is selected from the group consisting of: 3-(1,1-dimethyl-butyl)-6,6,9-trimethyl-6a,7,10,10a-tetrahydro-6H-benzo[c]chromene (JWH 133); L759633; L759656; {4-[4-(1,1-dimethyl-heptyl)-2,6-dimethoxy-phenyl]-6,6-dimethyl-bicyclo[3.1.1]hept-2-en-2-yl}-methanol (HU308); JWH015; (2-iodo-5-nitro-phenyl)-[1-(1-methyl-piperidin-2-ylmethyl)-1H-indol-3-yl]-methanone (AM-1241); and mixtures thereof.

22. The method of claim 13 wherein the CB₂ antagonist or the CB₂ inverse agonist is selected from the group consisting of: N-{1,3,3-Trimethyl-endo-(1S)-bicyclo[2.2.1]hept-2-yl}-1-[1-(4-methyl)-benzyl-5-(4-chloro-3-methyl-phenyl)-1H-pyrazol-3-carboxamide (SR-144528), JTE-907, AM630, and mixtures thereof.

23. The method of claim 13 wherein the compound having both CBₓ agonist and CB₂ agonist properties is selected from the group consisting of: 2-icosa-5,8,11,14-tetraenyloxy-propane-1,3-diol.

24. The method of claim 13 wherein the CBₓ modulator is selected from the group consisting of: 6,6,9-trimethyl-3-pentyl-6a,7,8,10a-tetrahydro-6H-benzo[c]chromen-1-ol; N-(Endo-bicyclo[2.2.1]hept-2-yl)-5-pentyl-4-phenyl-thiazole-2-carboxamide; 3-(1,1-dimethyl-butyl)-6,6,9-trimethyl-6a,7,10,10a-tetrahydro-6H-benzo[c]chromene; N-adamantyl-4-pentyl-5-phenyl-thiazole-2-carboxamide; 1-[1-Benzyl-5-(4-chloro-3-methyl-phenyl)-1H-pyrazol-3-yl]-ethanonyl-1,3,3-trimethyl-bi-cyclo[2.2.1]hept-2-ylamine; (2-iodo-5-nitro-phenyl)-[1-(1-methyl-piperidin-2-ylmethyl)-1H-indol-3-yl]-methanone; {4-[4-(1,1-Dimethyl-heptyl)-2,6-dimethoxy-phenyl]-6,6-dimethyl-bicyclo[3.1.1]hept-2-en-2-yl}-methanol; and mixtures thereof.

25. A method of modulating a K_{ATP} channel in a mammal comprising the steps of:
administering to a mammal in need thereof a CBₓ modulator;
wherein the CBₓ modulator is selected from the group consisting of: CB₁ agonists, CB₂ agonists, CB₂ partial agonists, CB₂ antagonists, CB₂ inverse agonists, compounds having both CB₁ agonist and CB₂ agonist properties, and mixtures thereof.

26. A method of increasing insulin secretion in mammals comprising the step of:
administering to a mammal in need thereof a pharmaceutically effective amount of a CBₓ modulator as a K_{ATP} channel modulator;
wherein the CBₓ modulator is selected from the group consisting of: CB₁ agonists; CB₂ agonists; CB₂ partial agonists; CB₂ antagonists; CB₂ inverse agonists; and dually acting compounds which are both a CB₁ agonist and a CB₂ agonist; and mixtures thereof.

27. A method for modulating the biological activity of a cannabinoid receptor comprising the step of: contacting a CBₓ receptor modulator with the cannabinoid receptor, wherein the CBₓ receptor modulator binds to the CBₓ receptor and a K_{ATP} channel is modulated.

28. A complex comprising a cannabinoid receptor and a CBₓ receptor modulator wherein the CBₓ receptor modulator and the cannabinoid receptor are in contact to form the complex whereby a K_{ATP} channel is modulated.

29. A method of identifying compounds that modulate a cannabinoid receptor comprising the step of: combining a test compound suspected of being a modulator of a cannabinoid receptor with COS-cells transiently expressing hamster SUR1 in the presence of [³H] glibenclamide and assessing the binding affinity of the test compound at sulfonylurea and K_{ATP} channel opening sites.

30. A method of identifying compounds that modulate a cannabinoid receptor comprising the step of: combining a test compound suspected of being a modulator of a cannabinoid receptor with COS-cells transiently expressing rat SUR2A in the presence of [³H]P1075 and assessing the binding affinity of the test compound at sulfonylurea and K_{ATP} channel opening sites.
